(19) United States
(12) Patent Application Publication
(10) Pub. No.: US 2025/0269022 A1
(43) Pub. Date: Aug. 28, 2025

(54) ASSESSING AND TREATING CANCER

(71) Applicant: Mayo Foundation for Medical Education and Research, Rochester, MN (US)

(72) Inventors: Saad J. Kenderian, Rochester, MN (US); Michelle J. Cox, Rochester, MN (US); Neil E. Kay, Golden Valley, MN (US)

(21) Appl. No.: 18/289,049

(22) PCT Filed: May 6, 2022
(86) PCT No.: PCT/US2022/028139

§ 371 Date: Oct. 31, 2023

Related U.S. Application Data
(60) Provisional application No. 63/222,748, filed on Jul. 16, 2021.
(60) Provisional application No. 63/185,325, filed on May 6, 2021.

(51) Int. Cl. A61K 40/11; A61K 31/16; A61K 31/325; A61K 31/336; A61K 31/4409; A61K 31/55; A61K 35/17; A61K 40/31; A61K 45/06; A61P 35/02

(52) U.S. Cl. CPC A61K 40/11; A61K 31/16; A61K 31/325; A61K 31/336; A61K 31/4409; A61K 31/55; A61K 35/17; A61K 40/31; A61K 45/06; A61P 35/02

(57) ABSTRACT

This document provides methods and materials for assessing cancer. For example, methods and materials that can be used to determine if a mammal (e.g., a human) having cancer is likely to be responsive to a cancer immunotherapy (e.g., a chimeric T cell therapy) are provided. In some cases, methods and materials for treating a mammal having cancer and identified as being likely to respond to a cancer immunotherapy (e.g., a chimeric T cell therapy) are also provided.

### CROSS-REFERENCE TO RELATED APPLICATIONS

This application is a National Stage application under 35 U.S.C. § 371 of International Application No. PCT/US2022/028139, having an International Filing Date of May 6, 2022, which claims the benefit of U.S. Patent Application Ser. No. 63/185,325, filed on May 6, 2021, and U.S. Patent Application Ser. No. 63/222,748, filed on Jul. 16, 2021. The disclosures of the prior applications are considered part of (and are incorporated by reference in) the disclosure of this application.

### SEQUENCE LISTING

This application contains a Sequence Listing that has been submitted electronically as an ASCII text file named “07039-2045WO1_SL.txt.” The ASCII text file, created on Jun. 22, 2022, is 2,079 bytes in size. The material in the ASCII text file is hereby incorporated by reference in its entirety.

### TECHNICAL FIELD

This document relates to methods and materials for assessing cancer. For example, the methods and materials provided herein can be used to determine if a mammal (e.g., a human) having cancer is likely to be responsive to one or more cancer immunotherapies (e.g., one or more chimeric antigen receptor (CAR) T cell therapies). In some cases, this document provides methods and materials for treating a mammal having cancer and identified as being likely to respond to one or more cancer immunotherapies (e.g., one or more CAR T cell therapies.

### BACKGROUND INFORMATION

CD19-directed chimeric antigen receptor T (CART19) cell therapy has resulted in remarkable outcomes in B cell malignancies (Neelapu et al., N. Engl. J. Med., 377:2531-2544 (2017); Porter et al., N. Engl. J. Med., 365:725-733 (2011); and Maude et al., N. Engl. J. Med., 378:439-448 (2018)). However, durable remissions are achieved in 30%-40% of patients, and most patients relapse within the first 1-2 years (Locke et al., Lancet Oncol., 20:31-42 (2019)). Similarly, the initial response rates are 70%-80% in patients with chronic lymphocytic leukemia (CLL; Siddiqi et al., Blood, 134 (Suppl 1): 503 (2019); and Gauthier et al., Biol. Blood Marrow Transplant, 25: S9-S10 (2019)), but the durable responses are only 20%-40% (Porter et al., Sci. Transl. Med., 7: 303ra139 (2015)).

### SUMMARY

This document provides methods and materials involved in assessing and/or treating mammals (e.g., humans) having cancer. In some cases, the methods and materials provided herein can be used to determine whether or not a mammal having cancer is likely to be responsive to one or more cancer immunotherapies (e.g., one or more CAR T cell therapies). For example, a sample (e.g., a blood sample) obtained from a mammal (e.g., a human) having cancer can be assessed for the presence or absence of a population of extracellular vesicles (EVs) where 10 percent or more (e.g., 10 percent, 15, percent, 20 percent, 25 percent, 30 percent, 35 percent, 40 percent, or more) of the EVs of the population are positive for a programmed death-ligand 1 (PD-L1) polypeptide (a PD-L1^{high} EV population; e.g., a circulating PD-L1^{high} EV population). When a population of EVs has 10 percent or more (e.g., 10 percent, 15, percent, 20 percent, 25 percent, 30 percent, 35 percent, 40 percent, or more) of the EVs of that population that are positive for PD-L1, the population can be referred to as a PD-L1^{high} EV population. When a population of EVs has less than 10 percent of the EVs of that population that are positive for PD-L1, the population can be referred to as a PD-L1^{low} EV population. The presence or absence of a PD-L1^{high} EV population can be used to determine whether or not the mammal is likely to respond to one or more cancer immunotherapies (e.g., one or more CAR T cell therapies). In some cases, this document also provides methods and materials for treating a mammal having cancer (e.g., a blood cancer) where the treatment is selected based, at least in part, on whether the mammal is identified as being likely to be responsive to one or more cancer immunotherapies (e.g., one or more CAR T cell therapies) as described herein.

The leukemic microenvironment is rich with EVs secreted by blood cancer cells. As demonstrated herein, PD-L1⁺ EVs from blood cancer cells can contain miroRNAs that can induce exhaustion of T cells, thereby rendering cancer immunotherapies such as CAR T cell therapies ineffective. As also demonstrated herein, the presence of a PD-L1^{high} EV population within a mammal (e.g., a human) can indicate that that mammal contains enough EVs carrying miroRNAs that can induce exhaustion of T cells and thereby render cancer immunotherapies such as CAR T cell therapies ineffective. The absence of a PD-L1^{high} EV population within a mammal (e.g., a human) can indicate that that mammal lacks the amount of T cell exhaustion-inducing microRNAs needed to induce meaningful exhaustion of T cells. In such cases, that mammal can be treated effectively with a cancer immunotherapy such as CAR T cell therapy.

As described herein, the presence or absence of a PD-L1^{high} EV population in a sample obtained from a mammal (e.g., a human) having cancer can be used to determine whether or not that cancer will be responsive to one or more cancer immunotherapies (e.g., one or more CAR T cell therapies). For example, the presence of a PD-L1^{high} EV population in a sample obtained from a mammal (e.g., a human) having cancer can be used to identify that mammal as having a cancer likely to induce T cell exhaustion in CAR T cells and as not being likely to be responsive to a CAR T cell therapy. In another example, the absence of a PD-L1^{high} EV population in a sample obtained from a mammal (e.g., a human) having cancer can be used to identify that mammal as having a cancer unlikely to induce T cell exhaustion in CAR T cells and as being likely to be responsive to a CAR T cell therapy.

Having the ability to determine whether or not a particular patient is likely to respond to a particular cancer treatment (e.g., a cancer immunotherapy such as a CAR T cell therapy) allows clinicians to provide an individualized approach to cancer patient care.

In general, one aspect of this document features methods for assessing a mammal having cancer. The methods can include, or consist essentially of, (a) detecting a presence or absence of a PD-L1^{high} EV population in a sample from a mammal having cancer; (b) classifying the mammal as not being likely to respond to an immunotherapy if the presence of the PD-L1^{high} EV population is detected; and (c) classifying the mammal as being likely to respond to the immunotherapy if the absence of the PD-L1^{high} EV population is detected. The mammal can be a human. The sample can be whole blood, serum, plasma, peripheral blood mononuclear cells (PBMCs), urine, cerebrospinal fluid (CSF), tissue samples, saliva, tears, or lymph. The cancer can be a CLL, a myeloid leukemia, a non-Hodgkin lymphoma, a Hodgkin lymphoma, a myeloproliferative neoplasm, a breast cancer, a colon cancer, a lung cancer, a pancreatic cancer, a head and neck cancer, a gastrointestinal malignancy, a liver cancer, a cholangiocarcinoma, a skin cancer, a melanoma, or a sarcoma. The cancer can be a CLL. The immunotherapy can be a CAR T cell therapy. The method can include classifying the mammal as not being likely to respond to the immunotherapy. The method can include classifying the mammal as being likely to respond to the immunotherapy. The PD-L1^{high} EV population can include greater than about 7,500 PD-L1⁺ EVs per μL of the sample.

In another aspect, this document features methods for treating a mammal having cancer. The methods can include, or consist essentially of, (a) identifying a mammal having cancer as lacking a PD-L1^{high} EV population in a sample obtained from the mammal; and (b) administering a cancer immunotherapy to the mammal. The mammal can be a human. The cancer can be a CLL, a myeloid leukemia, a non-Hodgkin lymphoma, a Hodgkin lymphoma, a myeloproliferative neoplasm, a breast cancer, a colon cancer, a lung cancer, a pancreatic cancer, a head and neck cancer, a gastrointestinal malignancy, a liver cancer, a cholangiocarcinoma, a skin cancer, a melanoma, or a sarcoma. The cancer can be a CLL. The cancer immunotherapy can be a CAR T cell therapy.

In another aspect, this document features methods for treating a mammal having cancer. The methods can include, or consist essentially of, (a) identifying a mammal having cancer as having a PD-L1^{high} EV population in a sample obtained from the mammal; and (b) administering a cancer treatment to the mammal, where the cancer treatment is not a cancer immunotherapy. The mammal can be a human. The cancer can be a CLL, a myeloid leukemia, a non-Hodgkin lymphoma, a Hodgkin lymphoma, a myeloproliferative neoplasm, a breast cancer, a colon cancer, a lung cancer, a pancreatic cancer, a head and neck cancer, a gastrointestinal malignancy, a liver cancer, a cholangiocarcinoma, a skin cancer, a melanoma, or a sarcoma. The cancer can be a CLL. The cancer treatment can include administering a chemotherapeutic agent to the mammal. The cancer treatment can include subjecting the mammal to a radiation therapy.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1I. Identification of CLL-derived extracellular vesicles (EVs) in patients with CLL. FIGS. 1A-1E) Dot plots showing total particle number (FIG. 1A) and EV levels (FIGS. 1B-1E) measured by nanoscale flow cytometry in platelet-poor plasma isolated from normal individuals (n=10) and CLL patients (n=50). FIGS. 1B-1E) A panel of fluorescent antibodies was used to enumerate levels of EVs for CD45⁺ (FIG. 1B), CD19⁺ (FIG. 1C), CD5⁺CD19⁺ (FIG. 1D), and PD-L1⁺ (FIG. 1E). Values represent number of EVs per microliter transformed in a logarithmic scale (Mann-Whitney test; error bars, SD). FIG. 1F) Correlation analysis of levels of CLL-derived CD5⁺CD19⁺ EVs and PD-L1⁺ EVs in CLL patients. Pearson correlation coefficient was calculated with a two-tailed p value. FIG. 1G) Western blot showing expression of three EV-enriched markers (TSG101, CD9, CD81) and PD-L1 in a panel of six EV lysates obtained from platelet-poor plasma of CLL patients. A second band at higher molecular weight was detected for PD-L1 that corresponds to a glycosylated form of the protein. FIG. 1H) Relative intensity of gel bands for total PD-L1 (left panel) and glycosylated PD-L1 (right panel). Levels of PD-L1 were increased by 1.4-fold (minimum [min]-maximum [max], 1.17-1.77). The glycosylated form of PD-L1 was markedly increased in patients having a PD-L1^{high} EV population with a 3.0-fold increase (min-max, 1.78-4.37) compared to patients having a PD-L1^{low} EV population. FIG. 1I) Correlation analysis of levels of total PD-L1 (left panel) and glycosylated PD-L1 (right panel) between western blot and nanoscale flow cytometric quantification methods. Pearson correlation coefficient was calculated with a two-tailed p value.

FIGS. 2A-2F. CLL-derived EVs induce a state of CART cell dysfunction. FIG. 2A and FIG. 2B) Inhibitory receptor expression on activated CART cells is upregulated by CLL-derived EVs. CART19 cells were co-cultured for 24 hours with JeKo-1 cells with different concentrations of EVs (*p<0.05, ** p<0.01, *** p<0.001, **** p<0.0001, one-way ANOVA; error bars, SEM; three biological and two technical replicates, two experiments). FIGS. 2C and 2D) CART19 cell antigen-specific proliferation and killing of CD19⁺ JeKo-1 cells were decreased in the presence of CLL-derived EVs (triangles) compared to controls (squares). EVs/CART19 cells at a 100:1 ratio were co-cultured for 6 hours and plated at a 5:1 effector-to-target ratio (E:T ratio) with JeKo-1 cells (**** p<0.0001, one-way ANOVA; error bars, SEM; three biological and two technical replicates, three experiments). FIGS. 2E) CART19 cell antigen-specific proliferation was further decreased in the presence of CLL-derived EVs at 1,000:1 and 10,000:1 compared to 100:1. EVs/CART cells were co-cultured for 6 hours and plated at a 5:1 E:T ratio with JeKo-1 cells (*p<0.05, **** p<0.0001, one-way ANOVA; error bars, SEM; three biological and two technical replicates, one experiment). FIG. 2F) Treatment of JeKo-1 cell xenografts with CART19 cells alone (squares) improved survival compared to CART19 cells co-cultured with CLL-derived EVs (triangles) or untransduced (UTD) T cells (circles). NOD-SCID-γ^{-/-} mice were engrafted with the CD19⁺ luciferase⁺ cell line JeKo-1 (1×10⁶ cells intravenous (i.v.} via tail vein injection), and engraftment was confirmed through bioluminescence imaging (total flux, photons [p]/s). Mice were randomized to treatment with (1) UTD T cells, (2) CART19 cells, and (3) CART19 cells co-cultured ex vivo with CLL-derived EVs for 6 hours prior to injection. All T cells were washed prior to injection. A single low dose of CART19 cells (2.5×10⁵) was injected to induce relapse (*p=0.0198, log-rank test; five mice per group).

FIGS. 3A-3G. EVs from CLL patients induce phenotypical, functional, and transcriptomic changes of exhaustion in T cells. FIG. 3A) CLL-derived EVs do not express E-cadherin. E-cadherin was measured on EVs derived from normal donor (ND) and CLL patients by nanoscale flow cytometry compared to measurements of CD19 on CLL-derived EVs (*p<0.05, one-way ANOVA; error bars, SEM; three to five biological replicates, two technical replicates, one experiment). FIG. 3B) CLL-derived EVs decrease E-cadherin CART cell antigen-specific proliferation. EVs/CART cells at a ratio of 100:1 were co-cultured for 6 hours and plated at an E:T ratio of 5:1 with the E-cadherin⁺ breast cancer cell line MCF-7 (**** p<0.0001, one-way ANOVA; error bars, SEM; three biological replicates, two technical replicates). The absolute number of live T cells significantly decreased when E-cadherin CART cells were co-cultured with MCF-7 cells in the presence of CLL-derived EVs (triangles) compared to E-cadherin CART cells co-cultured with MCF-7 alone (squares). The UTD negative control (circles) shows background proliferation. FIGS. 3C and 3D) CART19 cell transcriptome is modulated by CLL-derived EVs. CART19 cells were co-cultured with irradiated JeKo-1 cells for 24 hours at a ratio of 10:1, 1:1, or 0:1 EVs/CART19 cells and then isolated by magnetic sorting (three biological replicates, adjusted p value <0.05). Gene expression with 10:1 EVs/CART19 cells and 1:1 EVs/CART19 cells compared to CART19 cells alone. EVs increase the expression of AP-1 (FOS-JUN) and YY1. FIG. 3E) Principal component analysis of CART19 cell RNA-sequencing samples. Similar gene expression patterns were noted between both 1:1 EV/CART19 cell (blue circles) and 10:1 EVs/CART19 cells (red circles). FIG. 3F) Ingenuity Pathway Analysis predicts increased activation of the AP-1 pathway (FOS-JUN, orange) in CART19 cells co-cultured with CLL-derived EVs. FIG. 3G) Gene set enrichment analysis for significantly upregulated genes shows enrichment for pathways associated with CD4 (p=0.037) and CD8 (p=0.0033) T cell signaling as well as AP-1 transcription factors (p=0.0445) (highlighted bars, p<0.05).

FIGS. 4A-4C. CART cell dysfunction is facilitated by PD-L1⁺ CLL-derived EVs. FIGS. 4A and 4B) CART19 cells alone (squares) control tumor burden better compared to CART19 cells co-cultured ex vivo with a PD-L1^{high} CLL-derived EV population (triangles) (** p=0.0088, two-way ANOVA; error bars, SEM; five mice per group). NOD-SCID-γ^{-/-} mice engrafted with the CD19 luciferase⁺ cell line JeKo-1 Luc-ZsGreen (1×10⁶ cells i.v. via tail vein injection) and engraftment confirmed through bioluminescent imaging (total flux, p/s). Mice were then randomized for treatment with (1) UTD T cells, (2) CART19 cells, (3) CART19 cells co-cultured ex vivo with a PD-L1^{high} CLL-derived EV population for 6 hours prior to injection, or (4) CART19 cells co-cultured ex vivo with a PD-L1^{low} CLL-derived EV population for 6 hours prior to injection. A single low dose of CART19 cells (2.5×10⁵) was injected to induce relapse. Mice treated with UTD T cells (blue squares) had continued progression of disease. Mice treated with CART19 cells that were pre-cultured with a PD-L1^{low} CLL-EV population had a non-statistically significant impairment of anti-tumor activity (triangles). Mice treated with CART19 cells that were pre-cultured with a PD-L1^{high} CLL-EV population had significant impairment of anti-tumor activity. FIG. 4C) The ability of a CLL-derived PD-L1^{high} EV population to impair CART19 cells is not significantly reversed by PD-L1 blockade. CART19 cells were co-cultured for 6 hours with and without a PD-L1^{high} CLL-derived EV population (100:1 EV/CART cell ratio) and with and without anti-PD-L1 antibody. CD19⁺ JeKo-1 cells were added at an ET ratio of 5:1. CART19 cell antigen-specific proliferation was significantly impaired in the presence of a PD-L1^{high} CLL-derived EV population (p<0.01, two-way ANOVA). This inhibited CART19 cell antigen-specific proliferation did not improve following a co-culture with anti-PD-L1 antibody (n=11 biological replicates, two technical replicates, four experiments).

FIGS. 5A-5I. Nanoscale flow cytometric detection of EV subpopulations from platelet-poor plasma. FIG. 5A) Representative scatterplots of a polystyrene and silica bead mixture detected by nanoscale flow cytometry. Two fluorescent polystyrene bead populations PS110=110 nm and PS500=500 nm, and 5 silica bead populations (180, 240, 300, 590, and 880 nm). Left panel represents light-scatter detection with LALS in X-axis and SALS in Y-axis. Middle panel represents LALS in X-axis and fluorescence (FL488) in Y-axis. Right panel represents LALS in X-axis and bead count in Y-axis. FIGS. 5B-5I) Representative scatterplots of a platelet-poor plasma sample incubated with fluorescent antibodies against PD-L1 (FIG. 5B), CD5 (FIG. 5D), CD19 (FIG. 5F), CD45 (FIG. 5H) or antibody-matched isotypes. Gates represent events acquired as EVs positive for each marker. Antibody titration curves (FIGS. 5C, 5E, 5G, and 5I) were obtained from nanoscale flow cytometric detection of EVs from normal individual-derived platelet-poor plasma (n=2-4) incubated with increasing concentrations of antibodies or antibody-matched isotypes. Red arrows indicate antibody concentrations used for EV immunophenotyping of patient plasma.

FIGS. 6A-6D. Evaluation of the performance of nanoscale flow cytometric detection of PD-L1^{pos} EVs from patient plasma. FIG. 6A) Scatterplots of nanoscale flow cytometric detection of PD-L1^{pos} EVs isolated from 786-O cells overexpressing PD-L1-GFP. PD-L1-GFP-positive EVs were incubated with antibody-matched isotype (left panel) or anti-PD-L1 (middle panel). EVs isolated from PD-L1 knockout cells were used as negative control (right panel). FIG. 6B) Antibody titration curve for PD-L1 antibody using PD-L1⁺ EVs isolated from 7860 cells. A fixed concentration of approximately 500,000 PDL1⁺ EVs determined by nanoscale flow cytometry was used as reference. FIG. 6C) Linear regression model showing correlation between concentrations of PD-L1-GFP EVs and antibody-labeled PD-L1^{pos} EVs from dilutions of PD-L1-GFP EVs spiked-in 3 platelet-poor plasma (normal individuals). Coefficient of determination (r²) and one-tailed p-value test was performed. FIG. 6D) Representative scatterplots of nanoscale flow cytometric detection of PD-L1^{pos} EVs spiked-in a platelet-poor plasma. Scatterplots for GFP detection (upper row) and antibody detection (lower row) are shown.

FIGS. 7A-7B. CLL-derived EVs impact TCR-specific proliferation and inhibitory receptor modulation of CART19 cells. FIG. 7A) Inhibitory receptor expression on CART cells activated by CD3: CD28 beads is upregulated by CLL-derived EVs. CART19 were co-cultured for 24 hours with a 3:1 bead:cell ratio and 100:1 EV:CART (** p<0.01, one-way ANOVA; error bars, SEM; 3 biological and 2 technical replicates, 2 experiments). FIG. 7B) TCR-specific proliferation of CART19 is significantly decreased after 6 hours of co-culture with CLL-derived EVs. CART19 were co-cultured for 24 hours with a 3:1 bead:cell ratio and 100:1 EV:CART (**** p<0.0001, one-way ANOVA; error bars, SEM; 3 biological and 2 technical replicates, 2 experiments).

FIGS. 8A-8B. CLL B cells and JeKo-1 showed similar alteration of CART19 antigen-specific proliferation and inhibitory receptor expression. Example of antigen-specific proliferation (FIG. 8A) and CTLA-4 expression (FIG. 8B) on CART19 co-cultured with CLL-derived EVs for 24 hours with CLL B cells or JeKo-1. Experiments were performed with 3 biological replicates of CLL-derived EVs at 4 different doses and 2 technical replicates. Results between CLL B cells and JeKo-1 were comparable and thus JeKo-1 was used as a controlled proxy in the CLL model.

FIGS. 9A-9B. CLL-derived EVs significantly impact antigen-specific proliferation of CART19 cells after 6 hours of co-culture. FIG. 9A) EV concentration declines in the presence of CART19 or untransduced T (UTD) cells within 2-to-6 hours of co-culture. EVs, CART19 or UTD, and CLL B cells co-cultured at a 100:1:1 ratio. Percentage of EVs in suspension measured by nanoscale flow cytometry at 0, 2, 4, and 6 hours. FIG. 9B) Antigen-specific proliferation of CART19 is significantly decreased after 6 hours of co-culture with CLL-derived EVs. EVs were co-cultured with CART19 cells at a 100:1 ratio for 0, 2, 6, and 24 hours and then activated with the CD19⁺ cell line JeKo-1.

FIGS. 10A-10B. Gating strategy for flow cytometry. FIG. 10A) Gating strategy to measure CAR expression on T cells. Goat anti-mouse F(ab′) 2 antibody (GAM) was used with live/dead aqua to detect CAR expression on CART19 cells. Cells were gated on FSC/SSC followed by singlet discrimination and live cells. Negative gates for CAR expression were set based on untransduced (UTD) T cells. FIG. 10B) Gating strategy to quantify CART19 cells and target cells. Cells were gated on FSC/SSC followed by singlet and live cell discrimination. CD3 and FSC were used to separate CART19 cells from target cells. Absolute quantification was performed using volumetric measurement. Calculations for both volumetric and bead quantification using CountBright beads are shown.

FIG. 11. CART19 cell therapy non-responders exhibit significantly more PD-L1⁺ EVs compared to responders prior to treatment. PD-L1⁺ EVs were enumerated from platelet-free plasma of baseline samples using nanoscale flow cytometry.

FIGS. 12A-12C. miRNA are significantly upregulated in antigen-activated CART19 cells co-cultured with CLL-derived EVs. FIG. 12A) miR185 (SEQ ID NO:2) and let-7 (SEQ ID NO:3) target AP-1-associated genes YY1, JUND, and YY1-associated factor 2 (YAF2) as predicted by TargetScan. Alignments shown include position 1097-1103 of YAF2 (SEQ ID NO:4) with residues of miR-185 (SEQ ID NO:2), position 2129-2135 of YY1 (SEQ ID NO:5) with residues of miR-185 (SEQ ID NO:2), position 2263-2269 of YY1 (SEQ ID NO:6) with residues of miR-185 (SEQ ID NO:2), position 4551-4558 of YY1 (SEQ ID NO: 7) with residues of miR-185 (SEQ ID NO:2), and position 1075-1082 of JUND (SEQ ID NO: 8) with residues of let-7 (SEQ ID NO:3). FIG. 12B) Three miRNA are significantly upregulated in activated CART19 cells when co-cultured with either 1:1 or 10:1 EV:CART19 cells. FIG. 12C) Expression of YY1 and JUNB are significantly upregulated in antigen-activated CART19 cells co-cultured with CLL-derived EVs at a 1:1 or 10:1 ratio.

FIG. 13. miR-185-3p inhibits CART19 cell killing. miRNA-185-3p mimics are introduced to the CART19 cells by lipofectamine (400, 200, 100, 20, 10, 5, and 0 pmol/well). Percent killing was significantly reduced at high doses of miR-185-3p.

FIGS. 14A and 14B. Engineering of CART cells preserve and enhance their antigen-specific proliferation. FIG. 14A) CART19 was co-cultured alone or with CLL-derived EVs for 6 hours and then activated with CD19⁺ cell line JeKo-1. Significant impairment of antigen-specific proliferation and upregulation of CTLA-4 is seen when the CARTs are co-cultured with CLL-EVs. But when the CLL-EVs are co-cultured with CART19-siRNA, the proliferation impairment and CTLA-4 upregulation is partially reversed. FIG. 14B) FOSL2-overexpressed CART19 show a significant increase in antigen-specific proliferation compared to wildtype CART19.

FIG. 15. miRNAs upregulated in EVs from PD-L1^{high} EV populations from non-responders.

FIG. 16. miRNAs downregulated in EVs from PD-L1^{high} EV populations from non-responders.

FIGS. 17A-17C. Pathway analysis of miRNAs in EVs from non-responders and responders shown as a gene set enrichment analysis (FIG. 17A), a volcano plot (FIG. 17B), and a heatmap (FIG. 17C).

FIGS. 18A-18C. Proliferation and cytotoxicity of CAR19 T cells treated with navitoclax (Navi). FIG. 18A) A proliferation assay. CART cells were pre-treated with navitoclax (Navi) at shown concentrations for 24 hours. The JeKo-1 cell line was co-cultured either in the absence of navitoclax (JeKo-1) or in the presence of navitoclax (JeKo-1+ Navi) for five days. P/I: PMA/Ionomycin. K002: CAR19 T cells with 4-1BB as costimulatory domain. FIGS. 18B and 18C) Cytotoxicity assays. JeKo-1 and navitoclax pre-treated CART cells were co-cultured with varying E:T ratios without (FIG. 18B) or with (FIG. 18) navitoclax, and the viability of JeKo-1 cells was measured in every 24 hours.

FIG. 19. A cytotoxicity assay showing an increase in CART cell activity over time compared to untreated CART cells longitudinal navitoclax treatment of CART cells. Cytotoxicity was evaluated at day 8 (D8), day 15 (D15), and day 21 (D21).

FIG. 20. Proliferation of CART cells decreases over time in longitudinal navitoclax treatment of CART cells. Proliferation was evaluated at day 8 (D8), day 15 (D15), and day 21 (D21). C322: Donor number for T cells. K122: CAR19 construct with a CD28 costimulatory domain.

FIGS. 21A-21C. EdU assay demonstrating that senescent cells have decreased cell cycle. Cycling was evaluated at day 8 (FIG. 21A), day 15 (FIG. 21B), and day 21 (FIG. 21C). C322: Donor number for T cells. K122: CART cells including a CD28 costimulatory domain.

FIGS. 22A-22B. Both venetoclax and navitoclax combination therapies with TsCART cells (CART cells that target TRAILshort) result in increased CART cell cytotoxicity against JeKo-1 cell line. Cytotoxicity of both CAR19 cells (FIG. 22A) and CARTS1 cells (FIG. 22B) was evaluated. D1: Donor 1. D2: Donor 2. Top horizontal line: affect of venetoclax alone on JeKo-1 cells. Bottom horizontal line: affect of navitoclax alone against JeKo-1 cells. Solid data lines indicate conditions without senolytics. Dotted data lines indicate conditions with a senolytic.

FIG. 23. CART cells combined with navitoclax resulted in decreased CART cell proliferation against JeKo-1 cells, while combination with venetoclax did not decreased proliferation. D1: Donor 1. D2: Donor 2. M: CART cells cultured in media alone. PI: PMA/Ionomycin. JeKo-1: CART cells co-cultured with JeKo-1 cell line. JeKo-1 Navi: CART cells co-cultured with JeKo-1 cell line in the presence of navitoclax. JeKo-1 Vene: CART cells co-cultured with JeKo-1 cell line in the presence of venetoclax.

FIGS. 24A-24C. D8 CART cells and D22 CART cells that were treated with navitoclax were transplanted into immunodeficient NSG mice, and two weeks later CART cells were introduced to the mice (FIG. 24). Navitoclax treatment did not improve CART cell activity at D22 (FIG. 24B). Survival is shown in FIG. 24C. Long-rank (Mantel-Cox) test (comparison C322 NO D8 vs C322 NO D22).

FIG. 25. A schematic of an in vitro model for CART exhaustion.

FIGS. 26A-26B. High levels of a eomesodermin (EOMES) polypeptide promoted T cell exhaustion. FIG. 26A) ingenuity pathway analysis show that T cell activation and differentiation pathways are the predominantly altered pathway following a co-culture with EVs. Z-scores are calculated based on the data set's correlation with the activated state. Z (standard score)=x (observed value)−mew (mean of the sample)/sigma (SD of the sample). FIG. 26B) Heatmap demonstrate a distinct transcriptomic signature when CART cells are exposed to EVs.

FIGS. 27A-27C. Leukemic EVs carry an inhibitory microRNA cargo. FIG. 27A) A heat map showing that 226 microRNA families were differentially expressed. FIG. 27B) Principal Component Analysis show separation of the signature associated with CLL-EV compared to normal EVs. FIG. 27C) The top 10 upregulated microRNAs in CLL-EVs compared to normal EVs and their reported targets.

FIG. 28. PDL1^{high} EVs are associated with lack of response in patients with lymphoma treated with CART19 cell therapy.

FIG. 29. Principal component analysis and heatmap of microRNA signature in non-responders compared to responders.

FIG. 30. Volcano plot of non-responders compared to responders highlighting the upregulated genes.

FIG. 31. Pathway enrichment analysis of non-responders compared to responders highlighting the significantly upregulated genes.

FIG. 32. miR-27b-3p gene targets.

FIG. 33. miR-28-3p gene targets.

FIG. 34. miR-29c-3p gene targets.

FIG. 35. miR-9-5p gene targets.

FIG. 36. Principal component analysis of microRNA signature 1 month after treatment with CART cell therapy of non-responders compared to responders.

FIG. 37. Volcano plot analysis of microRNA signature 1 month after treatment with CART cell therapy of non-responders compared to responders.

FIG. 38. miR-9-5p gene targets.

FIG. 39. let-7c-5p gene targets.

FIG. 40. miR-148b-3p gene targets.

FIG. 41. miR-126-3p gene targets.

FIG. 42. Principal component analysis and heatmap of microRNA signature 3 month after treatment with CART cell therapy of non-responders compared to responders.

FIG. 43. Volcano plot of microRNA signature 3 month after treatment with CART cell therapy of non-responders compared to responders.

FIG. 44. Pathway analysis of microRNA signature 3 month after treatment with CART cell therapy of non-responders compared to responders.

FIG. 45. miR-143-3p gene targets.

FIG. 46. miR-125b-5p gene targets.

FIG. 47. miR-193a-5p gene targets.

FIGS. 48A and 48B. Levels of the top 8 upregulated microRNAs over time (baseline, 1 month post CART, and 3 months post CART) in responders and non-responders. The most significantly upregulated microRNAs that match between CLL-EVs compared to normal EVs (FIG. 48A) to microRNAs that are upregulated in non-responders compared to responders (FIG. 48B).

FIG. 49. Expression of NFKB target genes in JeKo-1 cells co-cultured with CLL-EVs.

FIG. 50. Expression of NFKB target genes in CART19 co-cultured with CLL-EVs.

FIG. 51. FOSL2^{high} CART19 cells are less susceptible to inhibition in an in vitro model of CART cell exhaustion.

FIG. 52. FOSL2 overexpressing CART19 cells result in improved tumor control in xenograft mouse models. NSG mice were engrafted with the CD19+ luciferase+ JeKo-1 cells. One week following engraftment, mice underwent bioluminescence imaging (BLI) and then randomized to treated with CART19, FOSL2 overexpressing CART19, or control untransduced T cells. Mice were then followed with BLI to monitor disease control.

FIGS. 53A-53C. Combination of CART cell therapy with small molecules D-pantethine (FIG. 53A), imipramine (FIG. 53B), and fasudil (FIG. 53C).

### DETAILED DESCRIPTION

This document provides methods and materials involved in assessing and/or treating mammals (e.g., humans) having cancer. In some cases, the methods and materials provided herein can be used to determine whether or not a mammal having cancer is likely to be responsive to one or more cancer immunotherapies (e.g., one or more CAR T cell therapies). For example, a sample obtained from a mammal (e.g., a human) having cancer can be assessed for the presence or absence of a PD-L1^{high} EV population to determine whether or not the mammal is likely to respond to one or more cancer immunotherapies (e.g., one or more CAR T cell therapies). As described herein, the presence of a PD-L1^{high} EV population (e.g., circulating PD-L1^{high} EV population) within a sample obtained from a mammal (e.g., a human) having cancer can be used to determine that the mammal is likely to be responsive to one or more cancer immunotherapies (e.g., one or more CAR T cell therapies). This document also provides methods and materials for treating mammals (e.g., humans) having cancer (e.g., a blood cancer) where the treatment is selected based, at least in part, on whether the mammal is identified as being likely to be responsive to one or more cancer immunotherapies (e.g., one or more CAR T cell therapies) as described herein (e.g., based, at least in part, on the presence or absence of a PD-L1^{high} EV population within a sample obtained from the mammal). For example, a mammal (e.g., a human) having cancer and lacking a PD-L1^{high} EV population within a sample obtained from the mammal can be treated by administering one or more cancer immunotherapies (e.g., one or more CAR T cell therapies) to the mammal.

Any appropriate mammal having cancer can be assessed and/or treated as described herein. Examples of mammals that can have cancer and can be assessed as described herein (e.g., for the presence or absence of a PD-L1^{high} EV population) and/or treated as described herein (e.g., by administering one or more cancer immunotherapies such as one or more CAR T cell therapies to the mammal) include, without limitation, humans, non-human primates (e.g., monkeys), dogs, cats, horses, cows, pigs, sheep, mice, and rats. For example, a human having cancer can be assessed and/or treated as described herein.

A mammal (e.g., a human) having cancer that can be assessed as described herein (e.g., for the presence or absence of a PD-L1^{high} EV population) and/or treated as described herein (e.g., by administering one or more cancer immunotherapies such as one or more CAR T cell therapies to the mammal) can have any type of cancer. In some cases, a cancer can be a blood cancer. In some cases, a cancer can include one or more solid tumors. In some cases, a cancer can be a recurrent cancer. In some cases, a cancer can be a primary cancer. In some cases, a cancer can be a metastatic cancer. In some cases, a cancer can be a chemo-resistant cancer. Examples of cancers that a mammal can have such that the mammal can be assessed as described herein (e.g., for the presence or absence of a PD-L1^{high} EV population) and/or treated as described herein (e.g., by administering one or more cancer immunotherapies such as one or more CAR T cell therapies to the mammal) include, without limitation, leukemias (e.g., CLLs and myeloid leukemias), lymphomas (e.g., non-Hodgkin lymphomas and Hodgkin lymphomas), myeloproliferative neoplasms, breast cancers, colon cancers, lung cancers, pancreatic cancers, head and neck cancers, gastrointestinal malignancies, liver cancers, cholangiocarcinomas, skin cancers, melanomas, and sarcomas. A cancer can be any stage of cancer. In cases where a mammal has a CLL, the CLL can be any stage of CLL. For example, when a CLL is evaluated under the Rai system, the CLL can be any Rai stage (e.g., Rai stage 0, Rai stage I, Rai stage II, Rai stage III, or Rai stage IV). For example, when a CLL is evaluated under the Binet system, the CLL can be any Binet stage (e.g., Binet stage A, Binet stage B, or Binet stage C).

In some cases, the methods described herein can include identifying a mammal (e.g., a human) as having cancer. Any appropriate method can be used to identify a mammal as having a cancer. For example, imaging techniques and/or biopsy techniques can be used to identify mammals (e.g., humans) having cancer.

In some cases, methods described herein can include assessing a sample obtained from a mammal (e.g., human) having cancer for the presence or absence of a PD-L1^{high} EV population. As used herein, a PD-L1^{high} EV population refers to an EV population where 10 percent or more (e.g., 10 percent, 15, percent, 20 percent, 25 percent, 30 percent, 35 percent, 40 percent, or more) of the EVs of the population are positive for PD-L1, and a PD-L1^{low} EV population refers to an EV population where less than 10 percent of the EVs of the population are positive for PD-L1. Also as used herein, a PD-L1^{pos} EV population refers to an EV population where at least some of the EVs of the population are positive for PD-L1, and a PD-L1^{neg} EV population refers to an EV population where none of the EVs of the population are positive for PD-L1. A PD-L1^{pos} EV refers to an individual EV that is positive for PD-L1, and a PD-L1^{neg} EV refers to an individual EV that is negative for PD-L1. In some cases, a PD-L1^{high} EV population can be detected when a sample of plasma (e.g., platelet-poor plasma sample) is determined to contain greater than about 7,500 PD-L1^{pos} EVs per μL of sample, provided that the plasma sample contains less than 100,000 of total EVs per μL. In some cases, a PD-L1^{high} EV population can be detected in a human when a sample of plasma (e.g., platelet-poor plasma sample) is determined to contain greater than about 7,500 (e.g., greater than 8,000, greater than 9,000, greater than 10,000, greater than 11,000, greater than 12,000, greater than 13,000, greater than 14,000, or greater than 15,000) PD-L 1^{pos} EVs per μL of sample, provided that the plasma sample contains less than 100,000 of total EVs per μL. When using a plasma sample such as a platelet-poor plasma sample obtained from a human having a blood cancer (e.g., CLL), the plasma sample typically contains about 50,000 to about 500,000 EVs per μL. In some cases, a PD-L1^{high} EV population can be detected as described in Example 1.

An EV can be any appropriate EV. In some cases, an EV can be an exosome. In some cases, an EV can be a microvesicle (MV). In some cases, an EV can be a CD19⁺ EV. As used herein, a CD19⁺ EV can be any EV that is positive for CD19 on its surface. In some cases, an EV (e.g., a PD-L1^{pos} EV) can contain one or more cargoes. Examples of cargoes that can be contained within an EV (e.g., a PD-L1^{pos} EV) include, without limitation, nucleic acids (e.g., microRNAs, mRNAs, and ncRNAs), polypeptides (e.g., enzymes), lipids, metabolites, organelles, and adhesion molecules. When an EV (e.g., a PD-L1^{pos} EV) contains one or more microRNAs, the microRNA(s) can be any appropriate microRNA. Examples of microRNAs that can be contained within an EV (e.g., a PD-L1^{pos} EV) include, without limitation, miR-155 microRNAs, miR-185 microRNAs (e.g., miR-185-3p), miR-199 microRNAs (e.g., miR-199a-3p and miR-199b-3p), miR-151 microRNAs (e.g., miR-151a-5p and miR-151b), miR-486-3p, miR-130b-3p, miR15b-5p, miR-7849-3p, miR-34a-5p, let-7 microRNAs (e.g., let-7d-3p), miR-15b-5p, miR-370-3p, miR-96-5p, miR-142-3p, miR-324-3p, miR-6741-5p, miR370-3p, miR-210-3p, miR-6805-5p, miR96-5p, miR-125a-5p, and miR142-3p. In some cases, a microRNA that can be contained within an EV (e.g., a PD-L 1^{pos} EV) can inhibit expression of a polypeptide that results in T cell exhaustion. Examples of polypeptides whose expression is needed to minimize T cell exhaustion include, without limitation, FOS like 2, AP-1 transcription factor subunit (FOSL2) polypeptides, FOS like 1, AP-1 transcription factor subunit (FOSL1) polypeptides, Jun polypeptides, Src homology region 2 domain-containing phosphatase-1 (SHP-1) polypeptides, and Src homology region 2 domain-containing phosphatase-2 (SHP-2) polypeptides.

Any appropriate method can be used to detect PD-L1^{pos} EVs, PD-L1^{neg} EVs, the presence or absence of a PD-L1^{high} EV population, and/or the presence or absence of a PD-L1^{low} EV population within a sample (e.g., a sample obtained from a mammal such as a human). For example, cytometry methods (e.g., flow cytometry such as cell sorting), spectrometry methods, antibody dependent methods (e.g., enzyme-linked immunosorbent assays (ELISAs), immunoprecipitation, immunoelectrophoresis, and/or western blotting methods can be used to detect PD-L1^{pos} EVs, PD-L1^{neg} EVs, the presence or absence of a PD-L1^{high} EV population, and/or the presence or absence of a PD-L1^{low} EV population within a sample (e.g., a sample obtained from a mammal such as a human). In some cases, PD-L1^{pos} EVs, PD-L1^{neg} EVs, the presence or absence of a PD-L1^{high} EV population, and/or the presence or absence of a PD-L11w EV population within a sample (e.g., a sample obtained from a mammal such as a human) can be detected without enriching the EVs within the sample. In some cases, the numbers of PD-L1^{pos} EVs and/or PD-L1^{neg} EVs within a sample (e.g., a sample obtained from a mammal such as a human) can be determined as described in Example 1 and Example 2. In some cases, the numbers of PD-L1^{pos} EVs and/or PD-L1^{neg} EVs within a sample (e.g., a sample obtained from a mammal such as a human) can be determined as described elsewhere (see, e.g., Théry et al., J. Extracell. Vesicles. 7:1535750 (2018) and Gomes et al., Thromb. Haemost., 118 (09): 1612-1624 (2018)). In some cases, the presence or absence of a PD-L1^{high} EV population and/or the presence or absence of a PD-L1^{low} EV population within a sample (e.g., a sample obtained from a mammal such as a human) can be determined as described in Example 1 and Example 2. In some cases, the presence or absence of a PD-L1^{high} EV population and/or the presence or absence of a PD-L1^{low} EV population within a sample (e.g., a sample obtained from a mammal such as a human) can be determined as described elsewhere (see, e.g., Théry et al., J. Extracell. Vesicles. 7:1535750 (2018) and Gomes et al., Thromb. Haemost., 118 (09): 1612-1624 (2018)).

Any appropriate sample from a mammal (e.g., a human) having cancer can be assessed as described herein (e.g., for the presence or absence of a PD-L1^{high} EV population within a sample obtained from a mammal such as a human). In some cases, a sample can be a biological sample. In some cases, a sample can contain one or more biological molecules (e.g., nucleic acids such as DNA and RNA, polypeptides, carbohydrates, lipids, hormones, and/or metabolites). Examples of samples that can be assessed as described herein include, without limitation, fluid samples (e.g., whole blood, serum, plasma, PBMCs, urine, and CSF), tissue samples, saliva, tears, and lymph. A sample can be a fresh sample, a fixed sample (e.g., EDTA plasma, citrate plasma, and heparinized plasma), or a frozen sample. In some cases, a sample can be a processed sample (e.g., an embedded sample such as a paraffin or OCT embedded sample, or processed to isolate or extract one or more biological molecules). For example, a blood (e.g., plasma) sample can be obtained from a mammal and can be assessed for the presence or absence of a PD-L1^{high} EV population.

In some cases, an EV fraction can be isolated from a sample obtained from a mammal (e.g., a human) having cancer and can be assessed for the presence or absence of a PD-L1^{high} EV population. Any appropriate method can be used to isolate an EV fraction from a sample. For example, sucrose gradient fractions can be used to isolate an EV fraction from a sample.

In some cases, the presence or absence of a PD-L1^{high} EV population within a sample (e.g., a sample obtained from a mammal such as a human) can be used to determine the function of T cells (e.g., CAR T cells such as CAR T cells administered in a CAR T-cell therapy) in a tumor microenvironment within a mammal (e.g., a human) having cancer. For example, the presence of a PD-L1^{high} EV population within a sample (e.g., a sample obtained from a mammal such as a human) can indicate that a T cell will have reduced effector functions (e.g., increased susceptibility to exhaustion) in a tumor microenvironment within a mammal (e.g., a human) having cancer.

This document also provides methods and materials for treating mammals (e.g., humans) having cancer (e.g., a blood cancer) where the treatment is selected based, at least in part, on whether or not the mammal is identified as being likely to be responsive to one or more cancer immunotherapies (e.g., one or more CAR T cell therapies) as described herein (e.g., based, at least in part, on the presence or absence of a PD-L1^{high} EV population within a sample obtained from the mammal). In some cases, a mammal (e.g., a human) having cancer and assessed as described herein (e.g., to determine whether or not the mammal is likely to respond to one or more cancer immunotherapies based, at least in part, on the presence or absence of a PD-L1^{high} EV population within a sample obtained from the mammal) can be administered or instructed to self-administer one or more (e.g., one, two, three, four, five, or more) cancer treatments, where the one or more cancer treatments are effective to treat the cancer within the mammal. For example, a mammal having cancer can be administered or instructed to self-administer one or more cancer treatments selected based, at least in part, on whether or not the mammal is likely to respond to one or more cancer immunotherapies (e.g., based, at least in part, on the presence or absence of a PD-L1^{high} EV population within a sample obtained from the mammal).

When treating a mammal (e.g., a human) having cancer and identified as being likely to be responsive to one or more cancer immunotherapies (e.g., one or more CAR T cell therapies) as described herein (e.g., based, at least in part, on the absence of a PD-L1^{high} EV population within a sample obtained from the mammal), the mammal can be administered or instructed to self-administer one or more (e.g., one, two, three, four, five, or more) one or more cancer immunotherapies (e.g., one or more CAR T cell therapies). Examples of cancer immunotherapies include, without limitation, adoptive T cell therapies (e.g., CAR-T cell therapies such as CD19 directed CART cell therapies including tisagenlecleucel, axicabtagene ciloleucel; B-cell maturation antigen (BCMA) directed CART cell therapies, CD30 directed CART cell therapies, CD33 directed CART cell therapies, CD123 directed CART cell therapies, CLL1 directed CART cell therapies, HER2 directed CART cell therapies, c-met directed CART cell therapies, CD2 directed CART cell therapies, CD5 directed CART cell therapies, and CD7 directed CART cell therapies) antibody-based therapies (e.g., BiTE therapies such as blinatumumab, solitomab, and BCMA-BITE), mesothelin directed CART cell therapies, kappa or lambda CART cell therapies, Ig directed CART cell therapies, CEA CART cell therapies, solid tumor directed CART cell therapies, folate receptor alpha or beta directed CART cell therapies, and FGFR directed CART cell therapies. A cancer immunotherapy can target any appropriate cancer antigen. Examples of cancer antigens that can be targeted by a cancer immunotherapy include, without limitation, CD19, CD20, CD47, epithelial cell adhesion molecule (EpCAM), CD33, CD123, CLL1, CD5, CD7, CD2, CD22, c-MET, TROP2, CEA, E-Cadherin, c-kit, ROR1, folate receptor (e.g., folate receptor alpha (FRa) and folate receptor beta (FRB)), FGFR, EGFR, and HER2.

In some cases, when treating a mammal (e.g., a human) having cancer and identified as being likely to be responsive to one or more cancer immunotherapies (e.g., one or more CAR T cell therapies) as described herein (e.g., based, at least in part, on the absence of a PD-L1^{high} EV population within a sample obtained from the mammal), one or more cancer immunotherapies (e.g., one or more CAR T cell therapies) can be only cancer treatment administered to the mammal.

In some cases, when treating a mammal (e.g., a human) having cancer and identified as being likely to be responsive to one or more cancer immunotherapies (e.g., one or more CAR T cell therapies) as described herein (e.g., based, at least in part, on the absence of a PD-L1^{high} EV population within a sample obtained from the mammal), the mammal also can be treated with one or more additional agents/therapies used to treat cancer. Examples of additional agents/therapies used to treat cancer include, without limitation, surgery, radiation therapies, chemotherapies, targeted therapies (e.g., monoclonal antibody therapies), hormonal therapies, angiogenesis inhibitors, immunosuppressants, checkpoint blockade therapies (e.g., anti-PD-1 antibody therapy, anti-PD-L1 antibody therapy, and/or anti-CTLA4 antibody therapy), and/or bone marrow transplants. In cases where one or more cancer immunotherapies are used in combination with one or more additional agents/therapies, the one or more additional agents/therapies can be administered at the same time or independently. For example, one or more cancer immunotherapies can be administered first, and the one or more additional agents/therapies can be administered second, or vice versa.

When treating a mammal (e.g., a human) having cancer and identified as not being likely to be responsive to one or more cancer immunotherapies (e.g., one or more CAR T cell therapies) as described herein (e.g., based, at least in part, on the presence of a PD-L1^{high} EV population within a sample obtained from the mammal), the mammal can be administered or instructed to self-administer one or more (e.g., one, two, three, four, five, or more) immunotherapies including T cells that are (e.g., that are designed to be) resistant to the T cell exhaustion induced by a PD-L1^{high} EV population. For example, T cells (e.g., CAR T cells) can be engineered to overexpress one or more of the polypeptides that have their expression inhibited or reduced via the microRNAs present within a PD-L1^{high} EV population that induce T cell exhaustion. Examples of polypeptides that can be overexpressed by T cells being used to treat cancer (e.g., CAR T cells) to reduce or prevent T cell exhaustion of those T cells by a PD-L1^{high} EV population include, without limitation, FOSL2 polypeptides, Jun polypeptides, FOSL1 polypeptides, FOXP1 polypeptides, mTOR polypeptides, PPP2R5C polypeptides, VEGFA polypeptides, GRB2 polypeptides, IFNG polypeptides, JUN polypeptides, KPNA3 polypeptides, HDAC1 polypeptides, MAP2K1 polypeptides, MAP2K3 polypeptides, RAF1 polypeptides, SMAD4 polypeptides, BCL2 polypeptides, BCL2L2 polypeptides, CCNE1 polypeptides, ASXL2 polypeptides, CCND1 polypeptides, CCND3 polypeptides, CCNE1 polypeptides, CDC25A polypeptides, CDK6 polypeptides, DMTF1 polypeptides, E2F5 polypeptides, SIRT1 polypeptides, and SQSTM1 polypeptides. In some cases, a polypeptide that can be overexpressed by T cells being used to treat cancer (e.g., CAR T cells) to reduce or prevent T cell exhaustion of those T cells by a PD-L1^{high} EV population can be a polypeptide that is targeted by a miRNA listed as upregulated in Table 3.

In yet another example, T cells (e.g., CAR T cells) can be engineered to express a nucleic acid (e.g., RNA) that includes one or more (e.g., one, two, three, four, five, or more) miRNA target binding sites of the microRNAs present within a PD-L1^{high} EV population that induce T cell exhaustion. Such nucleic acids (e.g., RNAs) can function as microRNA sponges to absorb those miRNAs such that they have little ability to bind to a target gene and prevent expression of the encoded polypeptide. For example, nucleic acid encoding a nucleic acid (e.g., RNA) containing one or more (e.g., one, two, three, four, five, or more) miRNA target binding sites (e.g., a microRNA sponge) can be introduced into T cells such that the microRNA sponge is expressed.

In some cases, a microRNA sponge can bind (e.g., can bind and sequester) a single microRNA. For example, a microRNA sponge can be designed to include one, two, three, four, five, or more miRNA target binding sites for a single microRNA. In some cases, a microRNA sponge can bind (e.g., can bind and sequester) two or more (e.g., two, three, four, five, or more) different microRNAs. For example, a microRNA sponge can be designed to bind (e.g., bind and sequester) two or more different microRNAs. In some cases, a microRNA sponge can be designed to bind (e.g., bind and sequester) a set of different microRNAs of a particular miRNA family. Examples of microRNA sponges that can be expressed by T cells being used to treat cancer (e.g., CAR T cells) to reduce or prevent T cell exhaustion of those T cells by a PD-L1^{high} EV population include, without limitation, microRNA sponges that can bind (e.g., bind and sequester) let-7, microRNA sponges that can bind (e.g., bind and sequester) miR-155, microRNA sponges that can bind (e.g., bind and sequester) miR-185, microRNA sponges that can bind (e.g., bind and sequester) miR86, microRNA sponges that can bind (e.g., bind and sequester) miR34a, microRNA sponges that can bind (e.g., bind and sequester) miR15, microRNA sponges that can bind (e.g., bind and sequester) miR210, microRNA sponges that can bind (e.g., bind and sequester) miR142, microRNA sponges that can bind (e.g., bind and sequester) miR 15b, microRNA sponges that can bind (e.g., bind and sequester) miR 125a, and microRNA sponges that can bind (e.g., bind and sequester) miR130. In some cases, a microRNA sponge that can be expressed by T cells being used to treat cancer (e.g., CAR T cells) to reduce or prevent T cell exhaustion of those T cells by a PD-L1^{high} EV population can be a microRNA sponge that can target a miRNA listed as upregulated in Table 3. In some cases, when a T cell is a CAR T cell, a nucleic acid encoding a microRNA sponge can be included with the nucleic acid encoding the CAR expressed by the CAR T cell.

In another example, T cells (e.g., CAR T cells) can be engineered to overexpress one or more nucleic acids that induce RNA interference against the expression of one or more of the microRNAs present within a PD-L1^{high} EV population that induce T cell exhaustion. For example, T cells can be designed to include nucleic acid that can express one or more nucleic acid molecules designed to induce RNA interference against a microRNA contained within a PD-L1^{high} EV (e.g., miR-155 microRNAs, miR-185 microRNAs (e.g., miR-185-3p), miR-199 microRNAs (e.g., miR-199a-3p and miR-199b-3p), miR-151 microRNAs (e.g., miR-151a-5p and miR-151b), miR-486-3p, miR-130b-3p, miR 15b-5p, miR-7849-3p, miR-34a-5p, let-7 microRNAs (e.g., let-7d-3p), miR-15b-5p, miR-370-3p, miR-96-5p, and miR-142-3p). Examples of nucleic acid molecules that can induce RNA interference against a microRNA include, without limitation, siRNA molecules, shRNA molecules, and antisense molecules.

Any appropriate method can be used to express one or more nucleic acids (e.g., one or more nucleic acids that can encode a polypeptide whose expression is inhibited or reduced via the microRNAs present within a PD-L1^{high} EV population that induce T cell exhaustion, nucleic acid encoding a microRNA sponge, and/or nucleic acid that can induce RNA interference against the expression of one or more of the microRNAs present within a PD-L 1^{high} EV population that induce T cell exhaustion) in a T cell that can be administered to a mammal (e.g., a human) as described herein. In some cases, nucleic acid that can encode a polypeptide whose expression is inhibited or reduced via the microRNAs present within a PD-L1^{high} EV population that induce T cell exhaustion, nucleic acid encoding a microRNA sponge, and/or nucleic acid that can induce RNA interference against the expression of one or more of the microRNAs present within a PD-L1^{high} EV population that induce T cell exhaustion can be introduced into one or more T cells of a population of T cells to be administered to a mammal as described herein. For example, nucleic acid that can encode a polypeptide whose expression is inhibited or reduced via the microRNAs present within a PD-L1^{high} EV population that induce T cell exhaustion, nucleic acid encoding a microRNA sponge, and/or nucleic acid that can induce RNA interference against the expression of one or more of the microRNAs present within a PD-L1^{high} EV population that induce T cell exhaustion can be introduced into one or more T cells of a population of T cells to be administered to a mammal as described herein can be introduced into the T cells using one or more viral vectors. For example, nucleic acid that can encode a polypeptide whose expression is inhibited or reduced via the microRNAs present within a PD-L1^{high} EV population that induce T cell exhaustion, nucleic acid encoding a microRNA sponge, and/or nucleic acid that can induce RNA interference against the expression of one or more of the microRNAs present within a PD-L1^{high} EV population that induce T cell exhaustion can be introduced into one or more T cells of a population of T cells to be administered to a mammal as described herein can be introduced into the T cells using one or more non-viral vectors.

When a vector used to deliver nucleic acid that can encode a polypeptide whose expression is inhibited or reduced via the microRNAs present within a PD-L1^{high} EV population that induce T cell exhaustion, nucleic acid encoding a microRNA sponge, and/or nucleic acid that can induce RNA interference against the expression of one or more of the microRNAs present within a PD-L1^{high} EV population that induce T cell exhaustion to one or more T cells of a population of T cells to be administered to a mammal is a viral vector, any appropriate viral vector can be used. Examples of viral vectors that can be used to deliver nucleic acid that can encode a polypeptide whose expression is inhibited or reduced via the microRNAs present within a PD-L1^{high} EV population that induce T cell exhaustion, nucleic acid encoding a microRNA sponge, and/or nucleic acid that can induce RNA interference against the expression of one or more of the microRNAs present within a PD-L1^{high} EV population that induce T cell exhaustion to one or more T cells of a population of T cells to be administered to a mammal include, without limitation, lentiviral vectors, retroviral vectors, adenoviral vectors, adeno-associated virus (AAV) vectors, vesicular stomatitis virus (VSV) vectors, measles vectors, and cytomegalovirus (CMV) vectors.

When a vector used to deliver nucleic acid that can encode a polypeptide whose expression is inhibited or reduced via the microRNAs present within a PD-L1^{high} EV population that induce T cell exhaustion, nucleic acid encoding a microRNA sponge, and/or nucleic acid that can induce RNA interference against the expression of one or more of the microRNAs present within a PD-L1^{high} EV population that induce T cell exhaustion to one or more T cells of a population of T cells to be administered to a mammal is a non-viral vector, any appropriate non-viral vector can be used. In some cases, a non-viral vector can be an extracellular vesicle (e.g., exosome). In some cases, a non-viral vector can be an expression plasmid.

In addition to nucleic acid that can encode a polypeptide whose expression is inhibited or reduced via the microRNAs present within a PD-L1^{high} EV population that induce T cell exhaustion, nucleic acid encoding a microRNA sponge, and/or nucleic acid that can induce RNA interference against the expression of one or more of the microRNAs present within a PD-L1^{high} EV population that induce T cell exhaustion, a vector (e.g., a viral vector or a non-viral vector) can contain regulatory elements operably linked to the nucleic acid that can encode a polypeptide whose expression is inhibited or reduced via the microRNAs present within a PD-L1^{high} EV population that induce T cell exhaustion, nucleic acid encoding a microRNA sponge, and/or nucleic acid that can induce RNA interference against the expression of one or more of the microRNAs present within a PD-L1^{high} EV population that induce T cell exhaustion. Such regulatory elements can include promoter sequences, enhancer sequences, response elements, signal peptides, internal ribosome entry sequences, polyadenylation signals, terminators, or inducible elements that modulate expression (e.g., transcription or translation) of a nucleic acid. The choice of element(s) that may be included in a vector depends on several factors, including, without limitation, inducibility, targeting, and the level of expression desired. For example, a promoter can be included in a vector to facilitate transcription of a nucleic acid that can induce RNA interference against the expression of one or more of the microRNAs present within a PD-L 1^{high} EV population that induce T cell exhaustion. A promoter can be constitutive or inducible (e.g., in the presence of tetracycline), and can affect the expression of a nucleic acid encoding a polypeptide in a general or tissue-specific manner. Examples of promoters that can be used to drive expression of a nucleic acid that can encode a polypeptide whose expression is inhibited or reduced via the microRNAs present within a PD-L1^{high} EV population that induce T cell exhaustion, nucleic acid encoding a microRNA sponge, and/or nucleic acid that can induce RNA interference against the expression of one or more of the microRNAs present within a PD-L1^{high} EV population that induce T cell exhaustion in one or more T cells of a population of T cells to be administered to a mammal include, without limitation, U6, H1, and T7 promoters. As used herein, “operably linked” refers to positioning of a regulatory element in a vector relative to a nucleic acid in such a way as to permit or facilitate expression of the nucleic acid that can induce RNA interference against the expression of one or more of the microRNAs present within a PD-L1^{high} EV population that induce T cell exhaustion. For example, a vector can contain a promoter and nucleic acid that can induce RNA interference against the expression of one or more of the microRNAs present within a PD-L1^{high} EV population that induce T cell exhaustion. In this case, the promoter is operably linked to the nucleic acid that can induce RNA interference against the expression of one or more of the microRNAs present within a PD-L1^{high} EV population that induce T cell exhaustion such that it drives transcription in cells.

In yet another example, T cells (e.g., CAR T cells) can be engineered to replace one or more of the microRNA's target sequence(s) normally present within the genomic DNA of the T cell with a different nucleotide sequence that encodes the same amino acid sequence but lacks the microRNA's specific target sequence. For example, a let-7d-3p having the sequence CCUAGGAAGAGGUAGUAGGUUGCAUAGUUUUAGGGCAGGGAUUUUGCCCACA AGGAGGUAACUAUACGACCUGCUGCCUUUCUUAGG (SEQ ID NO:1) can bind a target sequence present in mRNA transcribed from a HMGA2 gene, a MEX3C gene, a YY1 gene, a HIF-1 gene, a RAS gene, and a ERB gene. T cells (e.g., CAR T cells) can be engineered to replace one or more of the let-7d-3p's target sequence(s) present within the genomic DNA of the T cell with a different nucleotide sequence that encodes the same amino acid sequence but lacks the microRNA's specific target sequence such that let-7d-3p cannot bind the mRNA. In such cases, the polypeptide that has its expression inhibited or reduced by that microRNA because of its presence in a PD-L1^{high} EV population can be expressed in the engineered T cell as it normally is without the risk of that expression being inhibited or reduced by the microRNA.

In yet another example, T cells (e.g., CAR T cells) can be engineered to reduce or eliminate expression of one or more of the polypeptides that have their expression increased via the microRNAs present within a PD-L1^{high} EV population that induce T cell exhaustion. Examples of polypeptides that T cells being used to treat cancer (e.g., CAR T cells) can be engineered to have reduced or eliminated expression of to reduce or prevent T cell exhaustion of those T cells by a PD-L1^{high} EV population include, without limitation, FOXO1 polypeptides, ACVR1B polypeptides, BCL21 polypeptides, PRKCA polypeptides, MAP2K7 polypeptides, CASP6 polypeptides, CASP7 polypeptides, CBX7 polypeptides, and CDKN2 polypeptides. In some cases, a polypeptide that T cells being used to treat cancer (e.g., CAR T cells) can be engineered to have reduced or eliminated expression of to reduce or prevent T cell exhaustion of those T cells by a PD-L1^{high} EV population can be a polypeptide that is targeted by a miRNA listed as downregulated in Table 3.

Any appropriate gene therapy technique can be used to engineer T cells (e.g., CAR T cells) to be resistant to the T cell exhaustion induced by a PD-L1^{high} EV population (e.g., to replace one or more of the microRNA's target sequence(s) normally present within the genomic DNA of the T cell with a different nucleotide sequence that encodes the same amino acid sequence but lacks the microRNA's specific target sequence and/or to reduce or eliminate expression of one or more of the polypeptides that have their expression increased via the microRNAs present within a PD-L1^{high} EV population that induce T cell exhaustion). Examples of gene therapy techniques that can be used to engineer T cells (e.g., CAR T cells) to be resistant to the T cell exhaustion induced by a PD-L1^{high} EV population (e.g., to replace one or more of the microRNA's target sequence(s) normally present within the genomic DNA of the T cell with a different nucleotide sequence that encodes the same amino acid sequence but lacks the microRNA's specific target sequence and/or to reduce or eliminate expression of one or more of the polypeptides that have their expression increased via the microRNAs present within a PD-L1^{high} EV population that induce T cell exhaustion) include, without limitation, gene replacement (e.g., using homologous recombination or homology-directed repair), gene editing (e.g., clustered regularly interspaced short palindromic repeat (CRISPR)/CRISPR-associated (Cas) nuclease (CRISPR/Cas), transcription activator-like effector nuclease (TALEN), or zinc finger nuclease gene editing techniques), microhomology repair, and non homology repair.

In some cases, CRISPR/Cas gene editing techniques can be used to engineer T cells (e.g., CAR T cells) to be resistant to the T cell exhaustion induced by a PD-L1^{high} EV population (e.g., to replace one or more of the microRNA's target sequence(s) normally present within the genomic DNA of the T cell with a different nucleotide sequence that encodes the same amino acid sequence but lacks the microRNA's specific target sequence and/or to reduce or eliminate expression of one or more of the polypeptides that have their expression increased via the microRNAs present within a PD-L1^{high} EV population that induce T cell exhaustion). CRISPR/Cas molecules are components of a prokaryotic adaptive immune system that is functionally analogous to eukaryotic RNA interference, using RNA base pairing to direct nucleic acid cleavage resulting in double stranded breaks (DSBs) about 3-4 nucleotides upstream of a protospacer adjacent motif (PAM) sequence (e.g., NGG). Directing nucleic acid DSBs with the CRISPR/Cas system requires two components: a Cas nuclease, and a guide RNA (gRNA) targeting sequence directing the Cas to cleave a target DNA sequence (Makarova et al., Nat Rev Microbiol, 9 (6): 467-477 (2011); and Jinek et al., Science, 337 (6096): 816-821 (2012)). The CRISPR/Cas system can be used in bacteria, yeast, humans, and zebrafish, as described elsewhere (see, e.g., Jiang et al., Nat Biotechnol, 31 (3): 233-239 (2013); Dicarlo et al., Nucleic Acids Res, doi: 10.1093/nar/gkt135, 2013; Cong et al., Science, 339 (6121): 819-823 (2013); Mali et al., Science, 339 (6121): 823-826 (2013); Cho et al., Nat Biotechnol, 31 (3): 230-232 (2013); and Hwang et al., Nat Biotechnol, 31 (3): 227-229 (2013)). A CRISPR/Cas system can include any appropriate Cas nuclease. Cas nucleases can be as described elsewhere (see, e.g., Shalem et al., 2014 Science 343:84-87; and Sanjana et al., 2014 Nature methods 11:783-784).

In some cases, a TALEN system can be used to engineer T cells (e.g., CAR T cells) to be resistant to the T cell exhaustion induced by a PD-L1^{high} EV population (e.g., to replace one or more of the microRNA's target sequence(s) normally present within the genomic DNA of the T cell with a different nucleotide sequence that encodes the same amino acid sequence but lacks the microRNA's specific target sequence and/or to reduce or eliminate expression of one or more of the polypeptides that have their expression increased via the microRNAs present within a PD-L1^{high} EV population that induce T cell exhaustion). Transcription activator-like (TAL) effectors are found in plant pathogenic bacteria of the genus Xanthomonas. These proteins play important roles in disease, or trigger defense, by binding host DNA and activating effector-specific host genes (see, e.g., Gu et al., Nature 435:1122-1125, 2005; Yang et al., Proc Natl Acad Sci USA 103:10503-10508, 2006; Kay et al., Science 318:648-651, 2007; Sugio et al., Proc Natl Acad Sci USA 104:10720-10725, 2007; and Römer et al., Science 318:645-648, 2007). Specificity depends on an effector-variable number of imperfect, typically 34 amino acid repeats (Schornack et al., J Plant Physiol 163:256-272, 2006; and WO 2011/072246). Polymorphisms are present primarily at repeat positions 12 and 13, which are referred to as the repeat variable-diresidue (RVD). The RVDs of TAL effectors correspond to the nucleotides in their target sites in a direct, linear fashion, one RVD to one nucleotide, with some degeneracy and no apparent context dependence. This mechanism for protein-DNA recognition enables target site selection and engineering of new TALENs with binding specificity for the selected sites. For example, an engineered TAL effector DNA binding domain targeting sequence can be fused to a nuclease to create a TALEN that can create nucleic acid DSBs at or near the sequence targeted by the TAL effector DNA binding domain. Directing nucleic acid DSBs with the TALEN system requires two components: a nuclease, and TAL effector DNA-binding domain directing the nuclease to a target DNA sequence (see, e.g., Schornack et al., J. Plant Physiol. 163:256, 2006). A TALEN system can include any appropriate nuclease. In some cases, a nuclease can be a non-specific nuclease. In some cases, a nuclease can function as a dimer. For example, when a nuclease that functions as a dimer is used, a highly site-specific restriction enzyme can be created. For example, each nuclease monomer can be fused to a TAL effector sequence that recognizes a different DNA target sequence, and only when the two recognition sites are in close proximity do the inactive monomers come together to create a functional enzyme. Examples of nucleases that can used in a TALEN system described herein include, without limitation, FokI, HhaI, HindIII, NotI, BbvCI, EcoRI, BglI, and AlwI. For example, a nuclease of a TALEN system can include a FokI nuclease (see, e.g., Kim et al. (1996) Proc. Natl. Acad. Sci. USA 93:1156-1160).

In some cases, a mammal (e.g., a human) having cancer and identified as not being likely to be responsive to one or more cancer immunotherapies (e.g., one or more CAR T cell therapies) as described herein (e.g., based, at least in part, on the presence of a PD-L1^{high} EV population within a sample obtained from the mammal) can be administered or instructed to self-administer one or more (e.g., one, two, three, four, five, or more) immunotherapies and can be administered or instructed to self-administer one or more (e.g., one, two, three, four, five, or more) agents that can reduce or eliminate EV production and/or EV trafficking. Examples of agents that can reduce or eliminate EV production and/or EV trafficking that can be administered to a mammal (e.g., a human) together with one or more immunotherapies include, without limitation, calpeptin, manumycin A, Y27632, D-pantethine, imipramine, fasudil, and GW4869. In some cases, an agent that can reduce or eliminate EV production and/or EV trafficking that can be administered to a mammal (e.g., a human) together with one or more immunotherapies can be as described elsewhere (see, e.g., Catalano et al., J. Extracell. Vesicles, 9 (1): 1703244 (2019)). In cases where one or more cancer immunotherapies are used in combination with one or more agents that can reduce or eliminate EV production and/or EV trafficking, the one or more agents that can reduce or eliminate EV production and/or EV trafficking can be administered at the same time or independently. For example, one or more cancer immunotherapies can be administered first, and the one or more agents that can reduce or eliminate EV production and/or EV trafficking can be administered second, or vice versa.

In some cases, a mammal (e.g., a human) having cancer and identified as not being likely to be responsive to one or more cancer immunotherapies (e.g., one or more CAR T cell therapies) as described herein (e.g., based, at least in part, on the presence of a PD-L1^{high} EV population within a sample obtained from the mammal) can be administered or instructed to self-administer one or more (e.g., one, two, three, four, five, or more) immunotherapies and can be administered or instructed to self-administer one or more (e.g., one, two, three, four, five, or more) agents that can reduce or eliminate miRNA induced CAR T cell inhibition. In some cases, an agent that can reduce or eliminate miRNA induced CAR T cell inhibition can be an mTOR inhibitor. Examples of mTOR inhibitors that can reduce or eliminate miRNA induced CAR T cell inhibition that can be administered to a mammal (e.g., a human) together with one or more immunotherapies include, without limitation, rapamycin, sirolimus, temsirolimus, everolimus, and ridaforolimus. In some cases, an agent that can reduce or eliminate miRNA induced CAR T cell inhibition can be an HDAC inhibitor. Examples of HDAC inhibitors that can reduce or eliminate miRNA induced CAR T cell inhibition that can be administered to a mammal (e.g., a human) together with one or more immunotherapies include, without limitation, vorinostat, belinostat, LAQ824, panobinostat, entinostat, tacedinaline, and mocetinostat. In some cases, an agent that can reduce or eliminate miRNA induced CAR T cell inhibition can be a checkpoint blocker (e.g., an immune checkpoint blocker such as a PD-1 inhibitor and a PD-L1 inhibitor). Examples of checkpoint blockers that can reduce or eliminate miRNA induced CAR T cell inhibition that can be administered to a mammal (e.g., a human) together with one or more immunotherapies include, without limitation, pembrolizumab, nivolumab, cemiplimab, atezolizumab, avelumab, and durvalumab. In some cases, an agent that can reduce or eliminate miRNA induced CAR T cell inhibition can be a senotherapeutic agent (e.g., a senolytic agent). Examples of senotherapeutic agents that can reduce or eliminate miRNA induced CAR T cell inhibition that can be administered to a mammal (e.g., a human) together with one or more immunotherapies include, without limitation, dasatinib, quercetin, navitoclax, and venetocalx. In some cases, an agent that can reduce or eliminate miRNA induced CAR T cell inhibition can reduce or eliminate signaling of a pathway that is listed as upregulated in Table 3. In cases where one or more cancer immunotherapies are used in combination with one or more agents that can reduce or eliminate miRNA induced CAR T cell inhibition, the one or more agents that can reduce or eliminate miRNA induced CAR T cell inhibition can be administered at the same time or independently. For example, one or more cancer immunotherapies can be administered first, and the one or more agents that can reduce or eliminate miRNA induced CAR T cell inhibition can be administered second, or vice versa.

In some cases, a mammal (e.g., a human) having cancer and identified as not being likely to be responsive to one or more cancer immunotherapies (e.g., one or more CAR T cell therapies) as described herein (e.g., based, at least in part, on the presence of a PD-L1^{high} EV population within a sample obtained from the mammal) can be administered or instructed to self-administer one or more (e.g., one, two, three, four, five, or more) immunotherapies and can be subjected to one or more therapies that can reduce or eliminate the number of circulating EVs in the blood of the mammal. Examples of therapies that can reduce or eliminate the number of circulating EVs in the blood of a mammal (e.g., a human) include, without limitation, apheresis (e.g., plasmapheresis, which is also known as plasma exchange or “plex”), ultrafiltration, and administration of one or more plasma adsorbents. In cases where one or more cancer immunotherapies are used in combination with one or more therapies that can reduce or eliminate the number of circulating EVs in the blood of a mammal (e.g., a human), the one or more therapies that can reduce or eliminate the number of circulating EVs in the blood of a mammal (e.g., a human) can be performed at the same time or independently. For example, one or more cancer immunotherapies can be administered before, during, and/or after one or more therapies that can reduce or eliminate the number of circulating EVs in the blood of a mammal (e.g., a human) are performed.

When treating a mammal (e.g., a human) having cancer and identified as not being likely to be responsive to one or more cancer immunotherapies (e.g., one or more CAR T cell therapies) as described herein (e.g., based, at least in part, on the presence of a PD-L1^{high} EV population within a sample obtained from the mammal), the mammal can be administered or instructed to self-administer one or more (e.g., one, two, three, four, five, or more) alternative cancer treatments (e.g., one or more cancer treatments that do not involve administering T cells). Examples of alternative cancer treatments that do not involve administering T cells and that can be used as described herein include, without limitation, administering one or more cancer drugs (e.g., chemotherapeutic agents, targeted cancer drugs, immunotherapy drugs, and hormones) and/or one or more immunomodulatory agents to a mammal in need thereof. Examples of cancer drugs that do not involve administering T cells and that can be administered to a mammal having cancer and identified as not being likely to respond to a cancer immunotherapy can include, without limitation, panobinostat, trichostatin A, trapoxin B, phenylbutyrate, valproic acid, vorinostat, belinostat, LAQ824, entinostat, tacedinaline, mocetinostat, GSK2141795, GSK2110183, VQD-002, perifosine, miltefosine, MK-2206, AZD5363, ipatasertib, pembrolizumab (e.g., KEYTRUDA®), lenvatinib mesylate (e.g., LENVIMA®), megestrol acetate, and combinations thereof. In some cases, an alternative cancer treatment can include surgery. In some cases, an alternative cancer treatment can include radiation therapies.

When treating a mammal (e.g., a human) having cancer as described herein, the treatment can be effective to reduce the severity of the cancer. In some cases, when a cancer is a CLL, the severity of CLL can be determined by the Rai system (e.g., Rai stage 0, Rai stage I, Rai stage II, Rai stage III, or Rai stage IV) and/or the Binet system (e.g., Binet stage A, Binet stage B, or Binet stage C). In some cases, the severity of cancer can be as described elsewhere (see, e.g., Parikh, 2018 Blood Cancer J. 8:93).

When treating a mammal (e.g., a human) having cancer as described herein, the treatment can be effective to reduce or eliminate the number of cancer cells present within the mammal. For example, the materials and methods described herein can be used to reduce the number of cancer cells present within a mammal having cancer by, for example, 10, 20, 30, 40, 50, 60, 70, 80, 90, 95, or more percent. For example, the materials and methods described herein can be used to reduce the size (e.g., volume) of one or more tumors present within a mammal having cancer by, for example, 10, 20, 30, 40, 50, 60, 70, 80, 90, 95, or more percent. In some cases, the number of cancer cells present within a mammal being treated can be monitored. Any appropriate method can be used to determine whether or not the number of cancer cells present within a mammal is reduced. For example, imaging techniques can be used to assess the number of cancer cells present within a mammal.

When treating a mammal (e.g., a human) having cancer as described herein, the treatment can be effective to improve survival of the mammal. For example, the materials and methods described herein can be used to improve the survival of a mammal having cancer by, for example, 10, 20, 30, 40, 50, 60, 70, 80, 90, 95, or more percent. For example, the materials and methods described herein can be used to improve the survival of a mammal having cancer by, for example, at least 6 months (e.g., about 6 months, about 8 months, about months, about 1 year, about 1.5 years, about 2 years, about 2.5 years, about 3 years, about 4 years, about 5 years, or more).

When treating a mammal (e.g., a human) having cancer as described herein, the treatment can reduce or eliminate administering a cancer treatment to the mammal that will be ineffective. For example, when a mammal is identified as not being likely to respond to one or more immunotherapies (e.g., based, at least in part, on the presence of a PD-L1^{high} EV population in a sample (e.g., a blood sample such as plasma) obtained from the mammal, the mammal is not administered one or more immunotherapies that are likely to be rendered exhausted.

The invention will be further described in the following examples, which do not limit the scope of the invention described in the claims.

### EXAMPLES

### Example 1: Leukemic Extracellular Vesicles Induce Chimeric Antigen Receptor T Cell Dysfunction in Chronic Lymphocytic Leukemia

Chimeric antigen receptor (CAR) T cell therapy has yielded unprecedented outcomes in some patients with hematological malignancies; however, inhibition by the tumor microenvironment has prevented the broader success of CART cell therapy. This Example investigates interactions between the tumor microenvironment and CART cells, and identifies an immunosuppressive microenvironment having an abundance of systemic extracellular vesicles (EVs) and a lower durable response rate to CART cell therapy in CLL.

### Results

Identification of CLL-Derived EVs in Patients with CLL

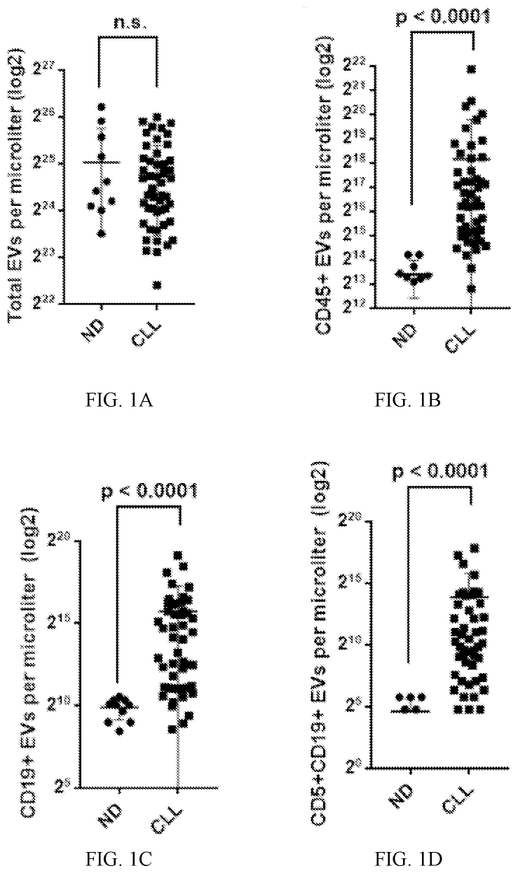

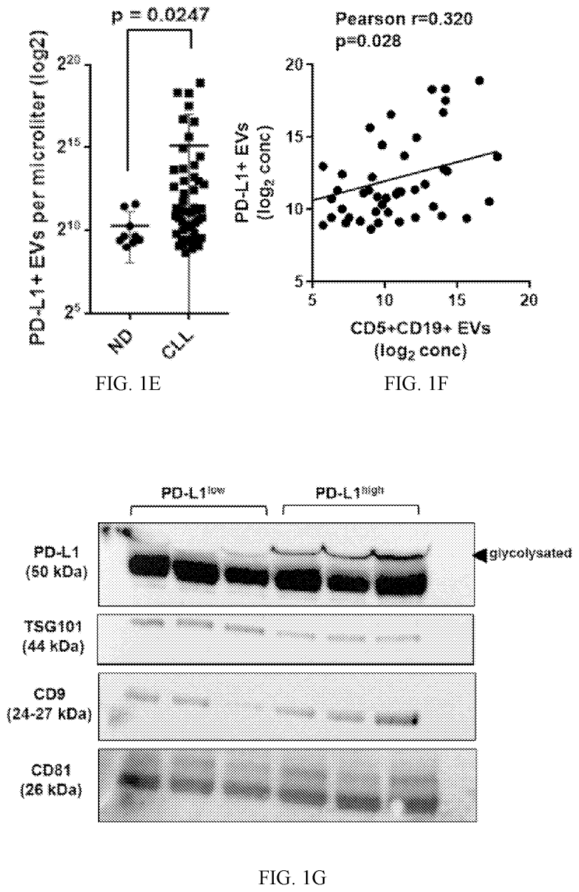

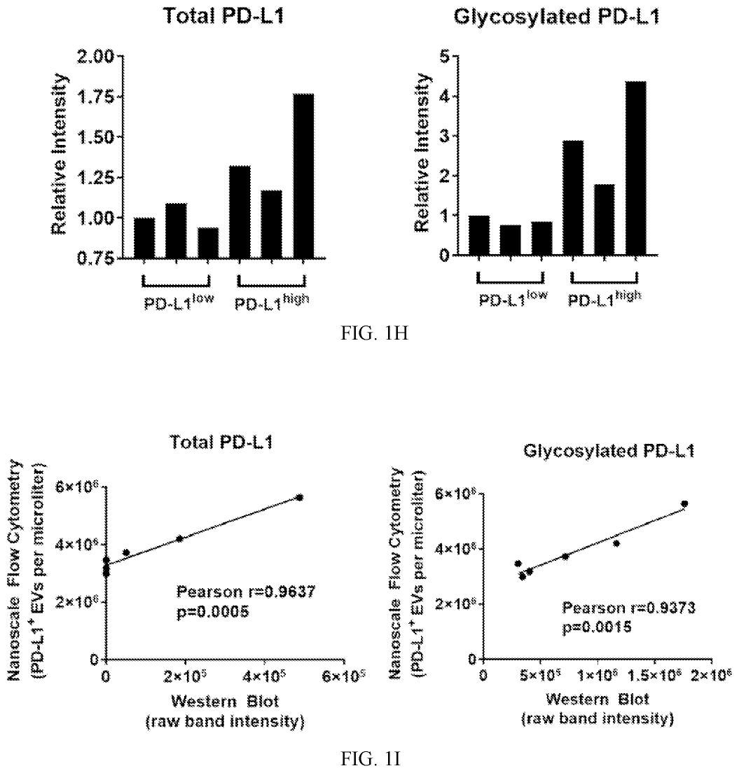

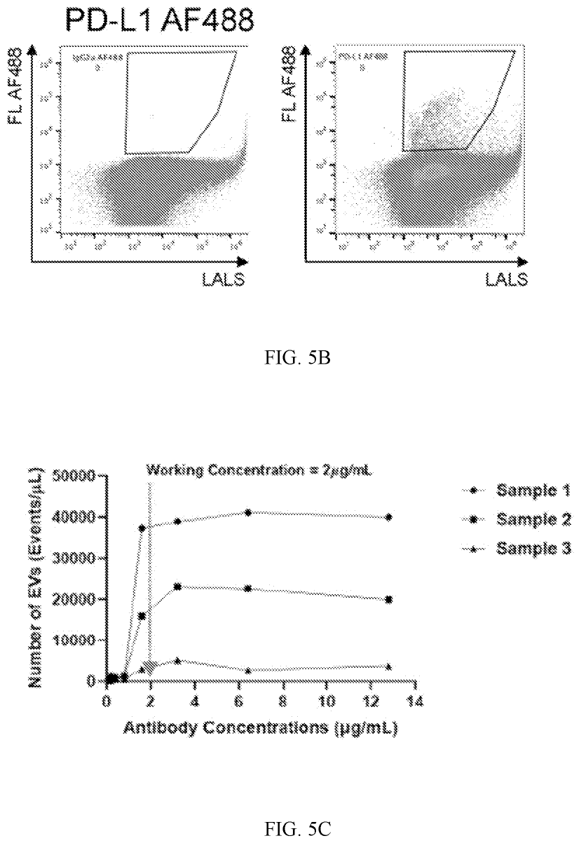

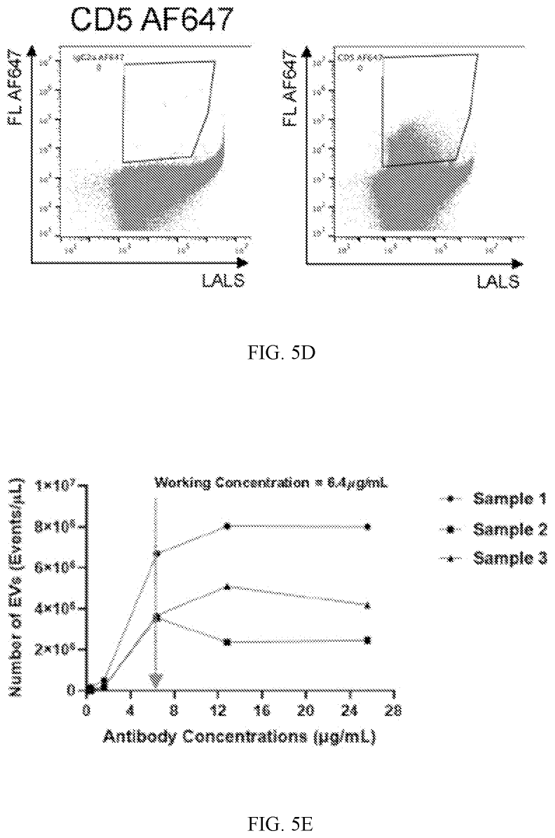

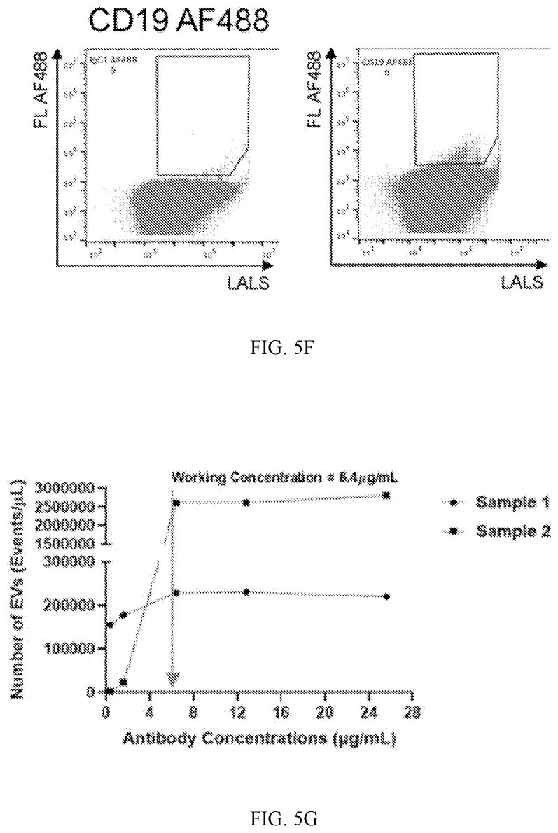

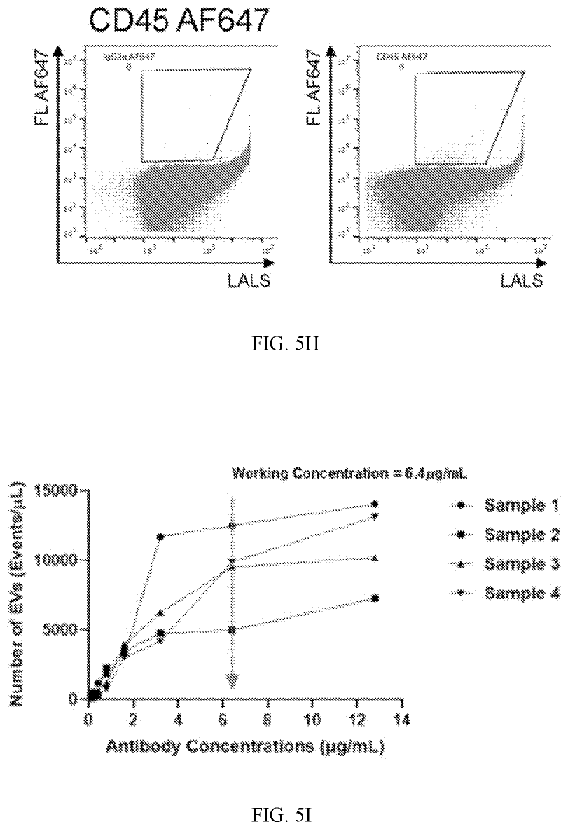

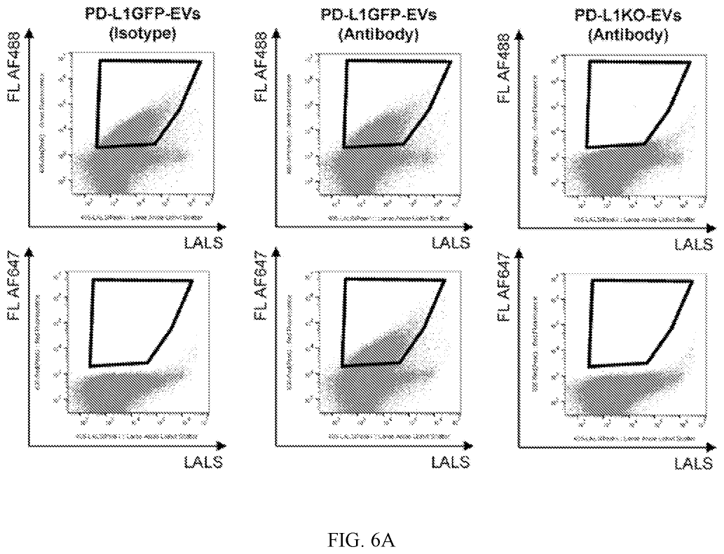

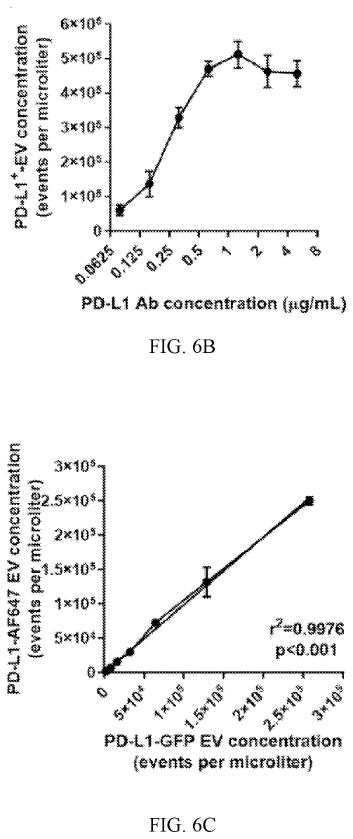

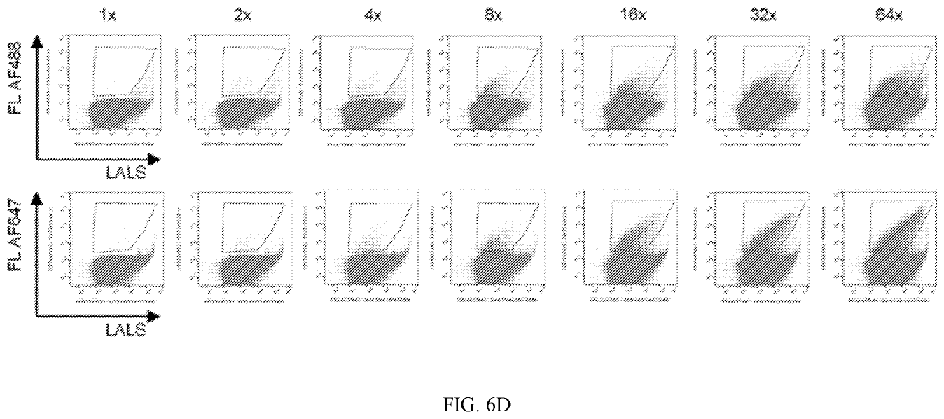

To isolate primary EVs from the plasma of CLL patients (Table 1), the guidelines of the International Society of Extracellular Vesicles (ISEV) established for the isolation of EVs from blood were used. To characterize and enumerate circulating EVs in platelet-poor plasmas of CLL patients, nanoscale flow cytometry, which allows for multiparametric detection of submicron particles using fluorescent antibodies, was used. Nanoscale flow cytometry allows for resolution of particles scattering light similarly to polystyrene and silica beads ranging from 110 to 1,000 nm (FIG. 5A). By using fluorescent antibodies, EVs can be characterized and enumerated for specific surface markers from platelet-poor plasma. Marker-positive EVs were detected by nanoscale flow cytometry with a size distribution in the same area as 110-nm polystyrene/180-nm silica beads and 300-nm silica beads (FIGS. 5B-5I). Total plasma particles and CD5⁺, CD19⁺, CD45⁺, and PD-L1⁺ EVs were enumerated from the plasma of CLL patients (n=50) and age-matched healthy individuals (n=10) (FIGS. 1A-1E). FIGS. 5B-5I depict the EV antigen expression and titration of the specific antibodies by nanoscale flow cytometry. As a positive control for PD-L1 expression on EVs, the PD-L1-GFP-expressing cell line 786-O was used to generate EVs expressing PD-L1. Linear quantification of PD-L1⁺ EVs was observed from concentrations ranging from 1,280 to 256,093 PD-L1⁺ EVs per microliter (FIG. 6).

TABLE 1
Characteristics of Untreated CLL Patients.

| ID | FISH | RAI Stage | IgVH Status | Sex | EV(×10⁶)/uL |
|---|---|---|---|---|---|
| 1 | 13q | 0 | unmutated | male | 30.2 |
| 2 | 13q | IV | mutated | male | 52.1 |
| 3 | 13q | 0 | unmutated | male | 48.1 |
| 4 | 13q | II | unmutated | male | 20.9 |
| 5 | 13q | 0 | — | female | 44.4 |
| 6 | — | I | — | male | 12.6 |
| 7 | 11q | 0 | unmutated | male | 20.9 |
| 8 | Normal | 0 | mutated | male | 18.4 |
| 9 | 11q | 0 | mutated | male | 38.8 |
| 10 | 13q | 0 | mutated | male | 23.2 |
| 11 | Normal | — | mutated | female | 14.2 |
| 12 | Normal | 0 | mutated | male | 10.8 |
| 13 | 13q | 0 | — | male | 9.1 |
| 14 | Normal | 0 | — | female | 26.8 |
| 15 | Tri-12 | — | mutated | female | 43.6 |
| 16 | — | 0 | — | male | 32.9 |
| 17 | 13q | 0 | mutated | male | 58.0 |
| 18 | — | I | — | female | 56.3 |
| 19 | — | 0 | — | female | 5.3 |
| 20 | 13q | 0 | — | male | 16.4 |
| 21 | — | I | unmutated | male | 13.4 |
| 22 | — | I | unmutated | male | 26.7 |
| 23 | 13q | 0 | uninterpretable | male | 12.4 |
| 24 | 13q | IV | mutated | female | 17.2 |
| 25 | Normal | IV | mutated | male | 27.9 |
| 26 | 13q | 0 | mutated | male | 20.6 |
| 27 | — | II | — | male | 30.3 |
| 28 | Normal | 0 | mutated | male | 17.4 |
| 29 | 13q | 0 | mutated | male | 36.2 |
| 30 | — | I | — | male | 16.8 |
| 31 | Normal | 0 | mutated | male | 20.3 |
| 32 | — | 0 | mutated | male | 62.4 |
| 33 | Normal | I | unmutated | male | 52.9 |
| 34 | 13q | 0 | unmutated | female | 18.6 |
| 35 | Tri-12 | 0 | unmutated | male | 21.7 |
| 36 | — | 0 | mutated | female | 33.1 |
| 37 | 13q | 0 | mutated | male | 19.3 |
| 38 | 13q | 0 | mutated | female | 66.6 |
| 39 | 13q | II | mutated | female | 27.5 |
| 40 | Normal | 0 | unmutated | male | 25.2 |
| 41 | 13q | 0 | mutated | female | 10.6 |
| 42 | 13q | 0 | unmutated | male | 31.0 |
| 43 | 13q | 0 | mutated | female | 10.8 |
| 44 | Normal | 0 | mutated | female | 10.0 |
| 45 | 13q | 0 | mutated | male | 29.3 |
| 46 | 13q | 0 | mutated | female | 17.1 |
| 47 | — | 0 | mutated | male | 50.7 |
| 48 | — | 0 | — | male | 34.6 |
| 49 | 13q | 0 | unmutated | male | 60.9 |
| 50 | Normal | 0 | unmutated | male | 130.0 |

No differences were observed for total particle counts in patients with CLL versus normal, age-matched controls (FIG. 1A). However, an abundance of CD45⁺ EVs (FIG. 1B), CD19⁺ EVs (FIG. 1C), and CD5⁺CD19⁺ EVs (FIG. 1D) were discovered in CLL patients. Given that CLL B cells are characterized as CD5⁺CD19⁺, the marked increase in levels of double-positive CD5⁺CD19⁺ EVs suggested CLL origins (FIG. 1D). Additionally, there was a significantly higher concentration of PD-L1⁺ EVs in CLL patients versus healthy controls (FIG. 1E). In CLL patients, levels of PD-L1⁺ EVs were positively correlated with levels of CLL-derived CD5⁺CD19⁺ EVs (Pearson r=0.320, p value=0.028) (FIG. 1F).

To further confirm the phenotype of EVs measured by nanoscale flow cytometry, six CLL patient plasmas with high EV particle counts were selected, and protein expression was measured by western blot. EVs were selected for further analysis from three patients having a PD-L1^{low} EV population and three patients having a PD-L1^{high} EV population. A combination of size-exclusion chromatography, ultracentrifugation, and lyophilization was used prior to lysis and protein extraction, and immunoblotting for three EV-enriched proteins: CD9, CD81, and TSG10123 was performed (FIG. 1G). All samples showed detectable levels for these three EV markers and confirmed the presence of PD-L1 in CLL patient-derived EVs (FIGS. 1G and 1H). Differential expression of PD-L1 was observed in the samples, and densitometry analysis revealed high and low levels of PDL1 expressing EVs in samples (FIG. 1I).

These results demonstrate that EV subpopulations can be detected and enumerated from platelet-poor plasma by nanoscale flow cytometry as verified by immunoblotting. Levels of CLL-derived CD5⁺CD19⁺ EVs were positively correlated with levels of PD-L1⁺ EVs in plasma of CLL patients, suggesting the presence of an immunosuppressive EV phenotype.

### CLL-Derived EVs Induce a State of CART Cell Dysfunction

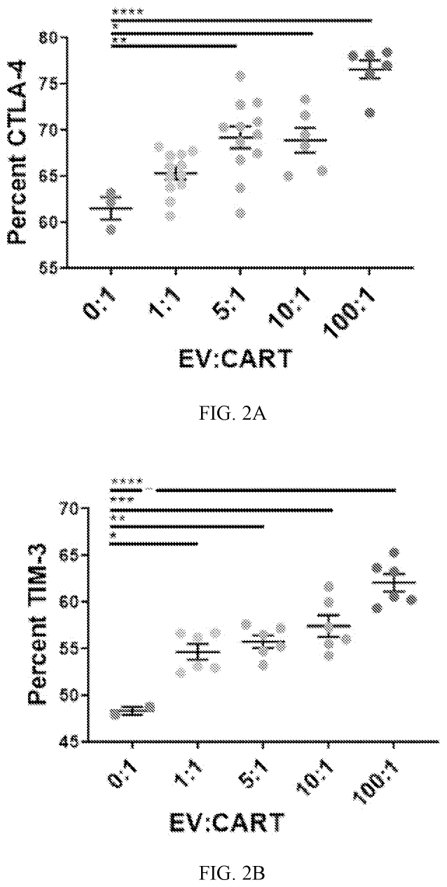

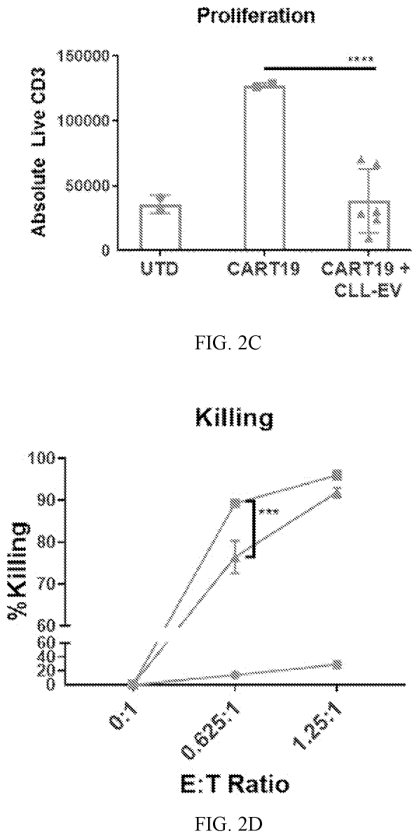

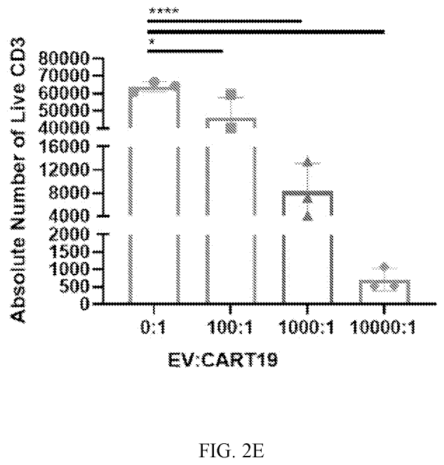

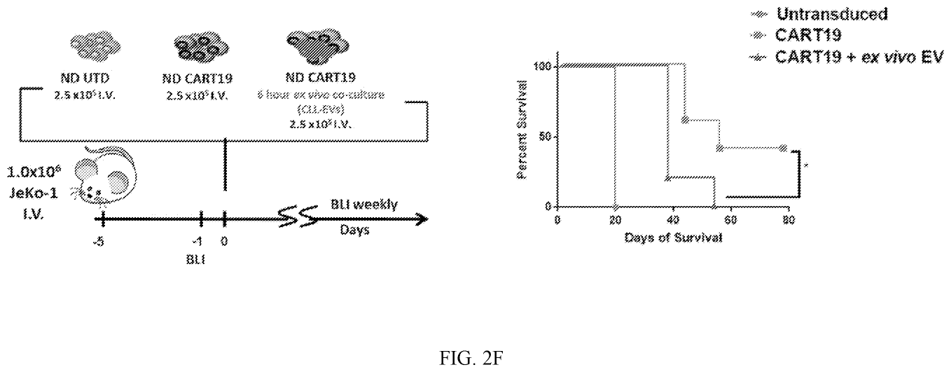

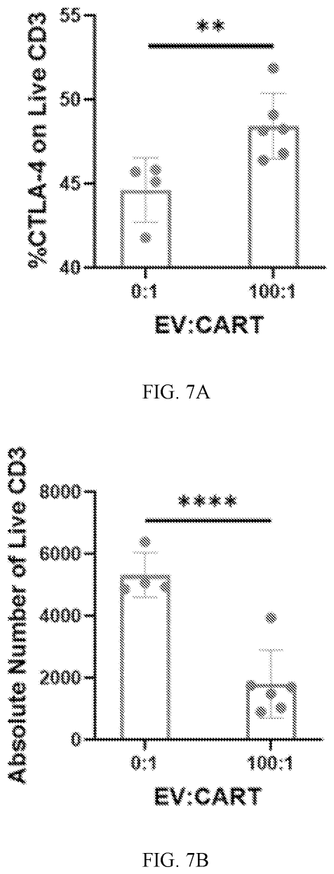

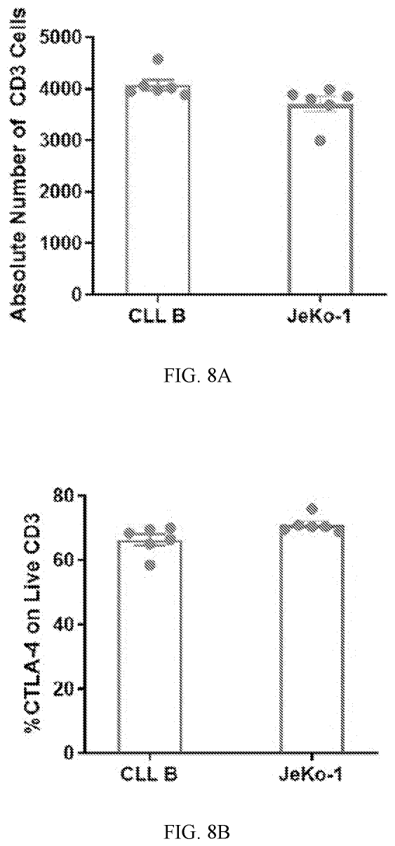

It was then sought to determine the direct effect of CLL-derived EVs on CART19 cell effector functions upon stimulation through the CAR with CD19⁺ target cells. To assess these effects, CART19 cells were cultured in increasing concentrations of EVs in platelet-poor plasma from CLL patients (CLL-derived EVs) with the CD19⁺ mantle cell lymphoma cell line, JeKo-1. A significant alteration of surface inhibitory receptors was detected, including increased expression of CTLA-4 and TIM-3 on activated CART19 cells, within 24 hours of EV co-culture (FIGS. 2A and 2B). Similar modulation of inhibitory receptors was also noted when CART19 cells were stimulated through their T cell receptor (TCR) with CD3/CD8 beads (FIG. 7). There was a significant impairment of CART19 cell antigen-specific proliferation (FIG. 2C) and antigen-specific killing (FIG. 2D) in the presence of CLL-derived EVs. A dose-dependent inhibition of CART cell antigen-specific proliferation was noted when CLL-derived EVs were co-cultured with CART19 cells. Inhibition was significant at EV/CART cell ratios of 100:1 (FIGS. 2C and 2D) and more profound when higher ratios were used (FIG. 2E). EV/CART cell ratios of 10,000:1 are closer to actual concentrations in patients treated with CART19 cell therapy. This inhibition of CART cell effector functions by CLL-derived EVs was also observed whether CART19 cells were stimulated with the CD19⁺ JeKo-1 cell line or with CD19⁺ leukemic cells isolated from CLL patients (FIG. 8). Therefore, JeKo-1 cells were used for the remaining experiments.

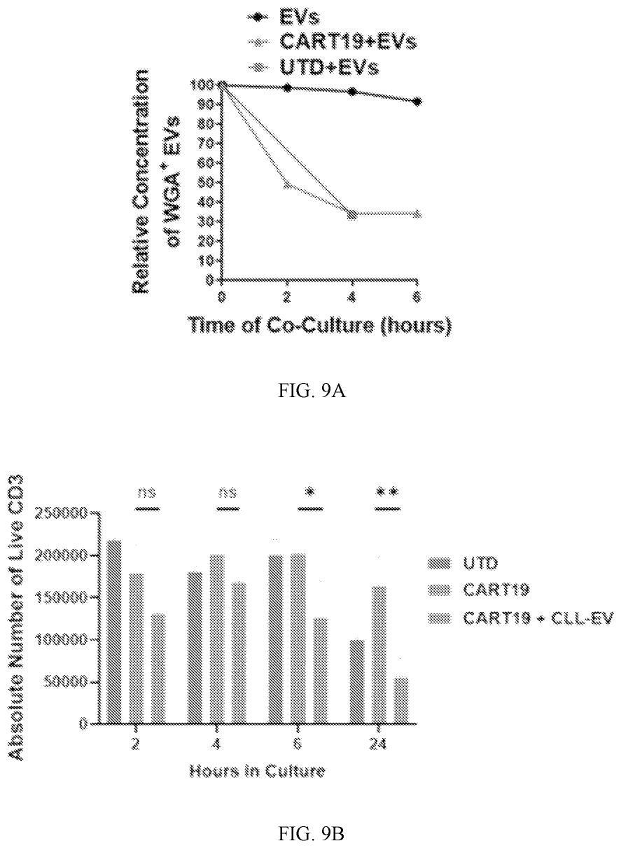

To validate these findings in vivo, an ex vivo co-culture of CLL-derived EVs with CART19 cells immediately prior to injection in a JeKo-1 xenograft model for relapsed disease was performed. The duration of co-culture required to induce CART19 cell dysfunction was first determined. Results indicate that EVs are taken by T cells within 4 hours (FIG. 9A) and that a co-culture of 6 hours is sufficient to suppress CART19 cell antigen-specific proliferation (FIG. 9B). Longer co-cultures led to more profound inhibition of CART cells. A 6-hour ex vivo co-culture was used as the most suitable co-culture setting for the in vivo experiment. In this JeKo-1 xenograft experiment, EV-exposed CART19 cells resulted in significantly decreased survival when compared to control CART19 cells (p=0.0198, FIG. 2F).

Evs from CLL Patients Induce Phenotypical, Functional, and Transcriptomic Changes of Exhaustion in T Cells

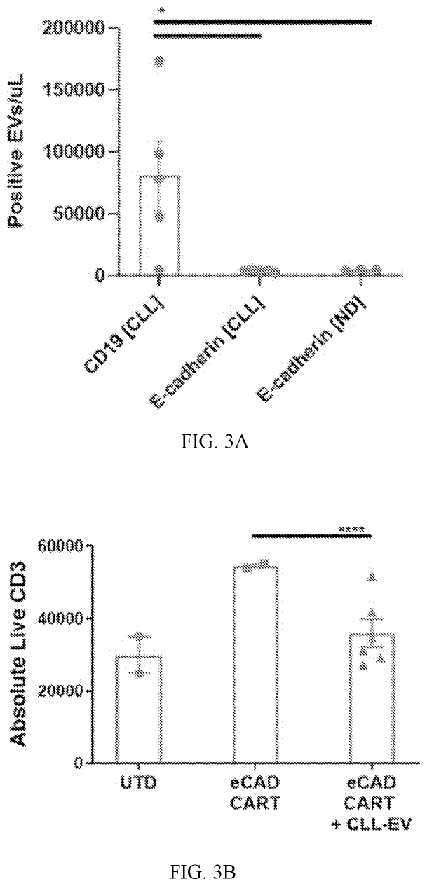

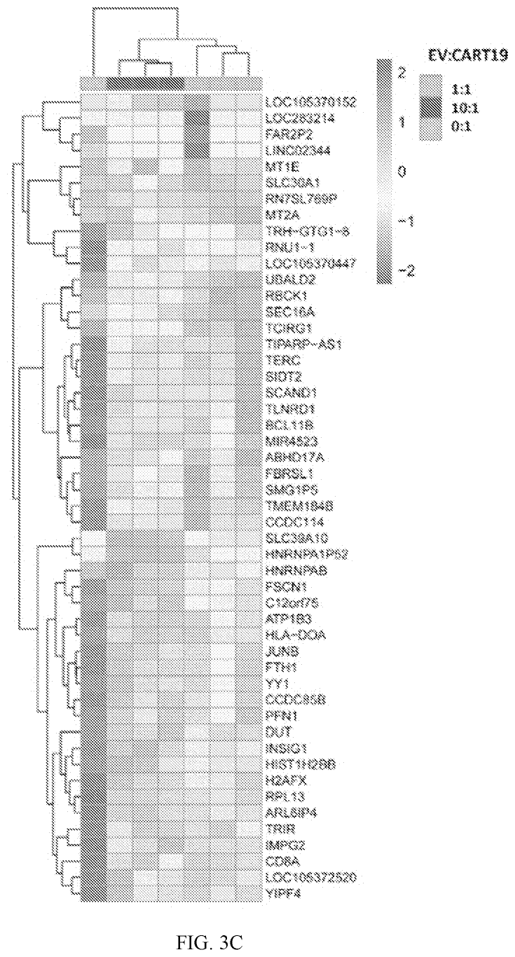

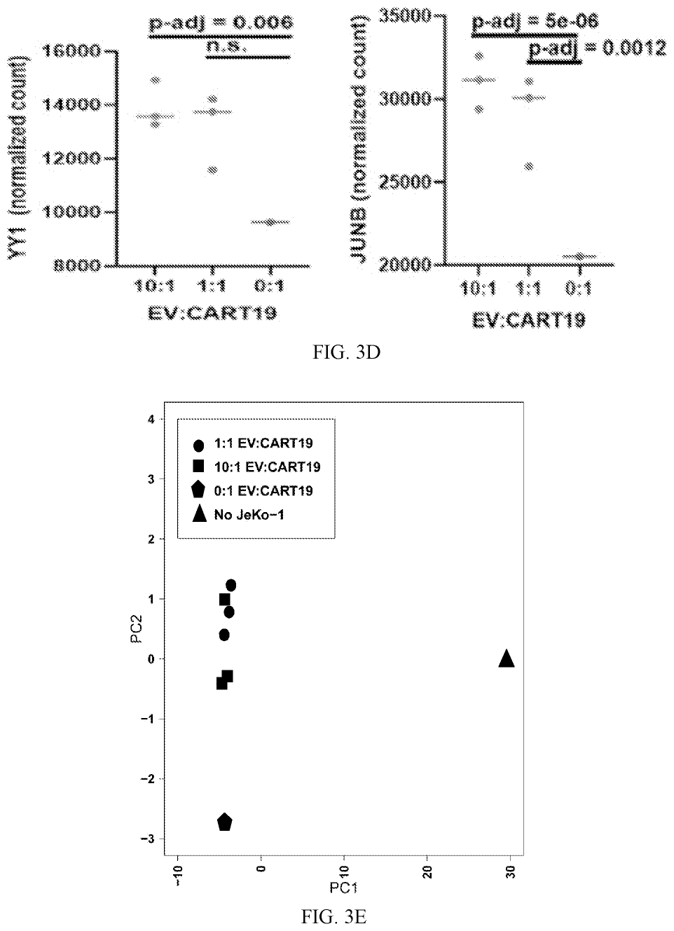

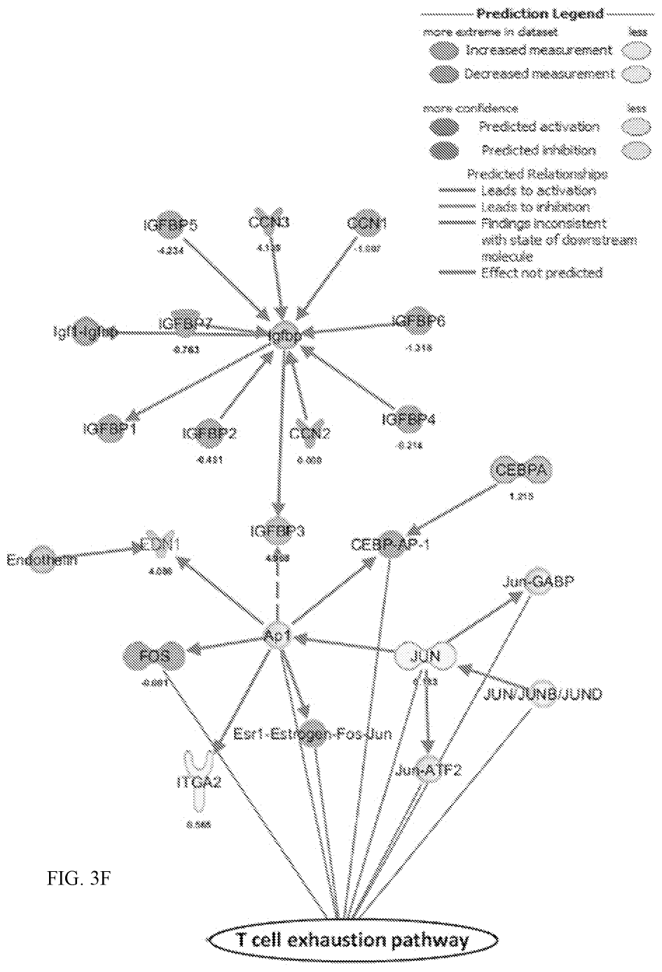

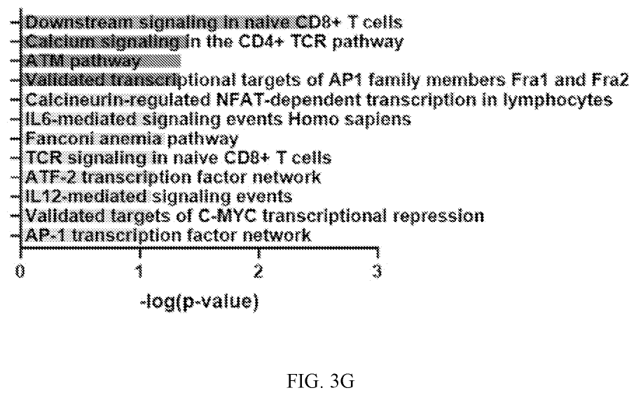

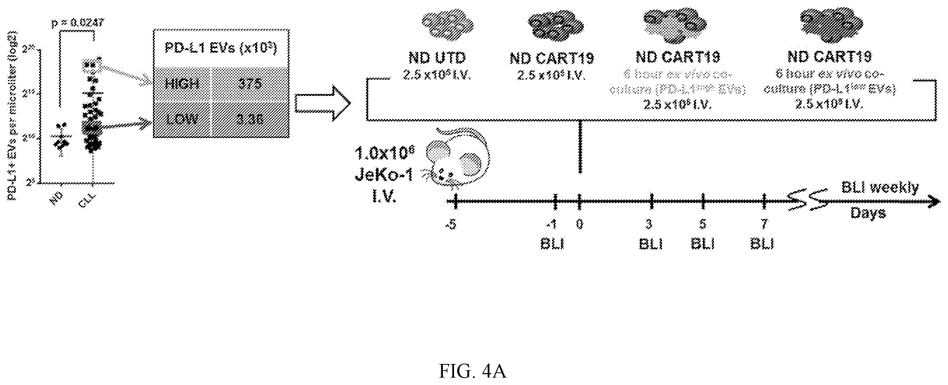

A potential mechanism for the impact of EVs on CART cells is a direct competition between the CD19⁺ EVs and the CD19⁺ tumor cells for the CD19-targeted single-chain variable fragment (scFv) on CART19 cells. To exclude effects from this potential competition, the modulation of E-cadherin-directed CART cell functions by CD19⁺ EVs was studied. The lack of E-cadherin expression on CLL-derived EVs was confirmed using nanoscale flow cytometry (FIG. 3A). EVs from CLL patients led to a significant inhibition of E-cadherin-directed CART cell antigen-specific proliferation in the presence of the E-cadherin⁺ cell line, MCF-7 (FIG. 3B). This suggested that CLL-EV-induced CART cell inhibition is not mediated by direct engagement of CART19 cell scFv with the CD19 ligand expressed on the surface of EVs.

To investigate whether EVs induce a state of CART cell dysfunction through modulation of exhaustion pathways, the transcriptome of stimulated CART19 cells in the presence or absence of CLL-derived EVs was evaluated. CART19 cells were stimulated through the CAR by co-culturing with irradiated JeKo-1 cells. Total RNA sequencing (RNA-seq) of activated CART19 cells highlighted a significant enhanced expression of AP-1 (FOS-JUN) and YY1 gene pathways in EV-exposed antigen-stimulated CART19 cells compared to antigen-stimulated CART19 cells alone (FIGS. 3C-3F). There were no clear differences between a high or low EV/CART19 cell ratio (FIG. 3D).

Gene set enrichment analysis was also performed on the significantly upregulated genes, which was highly robust for pathways such as CD4 and CD8 signaling and AP-1 transcriptional targets (FIG. 3G). These findings (FIGS. 2 and 3) suggest that EVs significantly induce known phenotypical, functional, and transcriptional hallmarks of T cell exhaustion.

### Cart Cell Dysfunction is More Specific to PD-L1⁺ CLL-Derived EVs

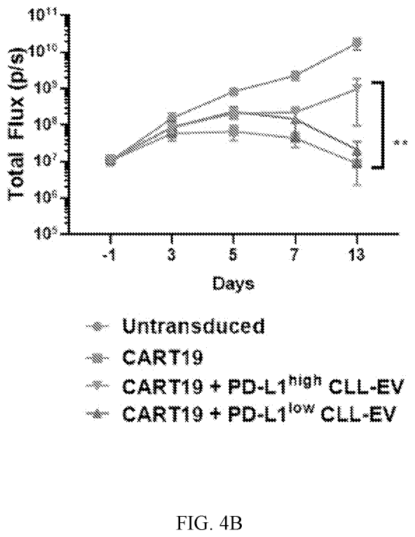

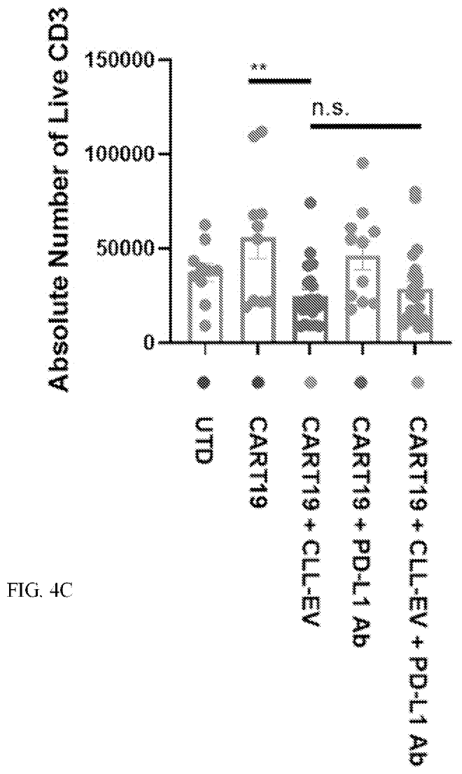

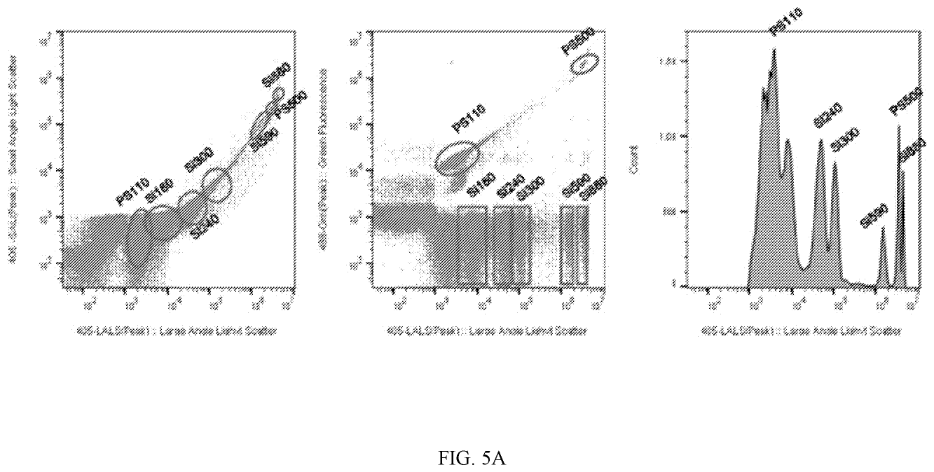

To determine the specific characteristics of CLL-derived EVs that resulted in CART cell dysfunction, the specific effects on anti-tumor efficacy of CART cells induced by PD-L1⁺ CLL-derived EVs were examined using a JeKo-1 xenograft model (FIGS. 4A and 4B). An ex vivo co-culture of CART19 cells with PD-L1^{high} CLL-derived EVs resulted in significantly inferior (p=0.0088) in vivo anti-tumor activity (FIG. 4B), whereas an ex vivo co-culture of CART19 cells with PD-L1^{low} CLL-derived EVs did not result in a significant impairment of anti-tumor activity. These experiments indicate that EV-induced CART cell dysfunction may be associated more specifically with PD-L1⁺ EVs. To examine whether the interaction between PD-L1 on CLL-derived EVs and PD-1 on CART cells is responsible for CART cell dysfunction, the antigen-specific proliferation of CART19 cells was measured in the presence of PD-L1^{high} CLL-derived EVs with or without PD-L1 blocking antibodies. There was no statistically significant reversal of EV-mediated inhibition of CART19 cells (FIG. 4C), suggesting that the interaction is not the predominant mechanism of CART cell dysfunction.

### Materials and Methods

### Preparation of Platelet-Poor Plasma for EVs

Platelet-poor plasma (PPP) samples were prepared following the International Society on Thrombosis and Hemostasis (ISTH), International Society for the Advancement of Science (ISAC), and ISEV recommendations (Théry et al., J. Extracell. Vesicles. 7:1535750 (2018)). Briefly, 10 mL of peripheral blood was collected in EDTA-coated vacutainers. Centrifugation was performed twice at 2,500×g at room temperature using lowest deceleration for 15 minutes to remove platelets and cellular debris. Plasma was aliquoted and stored at −80° C. These PPP preparations from the peripheral blood of untreated CLL patients are the source of the samples called CLL-derived EVs.

Immunophenotyping of Circulating EVs from Human Plasma

PPP samples were thawed at 37° C., and 10 μL of PPP was incubated with the following fluorescent antibodies or antibody-matched isotypes for 30 minutes at room temperature and in the dark: anti-CD45 (304002, BioLegend, San Diego, CA, USA), anti-CD5 (364002, Bio-Legend, San Diego, CA, USA), anti-CD19 (363002, BioLegend, San Diego, CA, USA), anti-PD-L1 (13684S, Cell Signaling Technology, Danvers, MA, USA), and anti-E-cadherin (147303, BioLegend, San Diego, CA, USA). Following EV labeling, samples were resuspended in filtered PBS (0.22 μm) and analyzed by nanoscale flow cytometry.

Optimal concentrations for each antibody were determined by antibody titration using two to four PPP samples (FIG. 5). All antibodies were conjugated with fluorescent dyes using antibody labeling kits (Thermo Fisher Scientific, Waltham, MA, USA) and according to the manufacturer's instructions. Final conjugated antibody concentration and degree of labeling were determined by using the Nano-Drop One (Thermo Fisher Scientific, Waltham, MA, USA).

### Nanoscale Flow Cytometry

All PPP samples were analyzed by using an A60-Micro-PLUS nanoscale flow cytometer (Apogee Flow Systems, Hemel Hempstead, Hertfordshire, UK). The A60-Micro-PLUS is equipped with a 405-nm laser for light-scatter measurement and two 488- and 638-nm lasers for fluorescence measurements. Before sample analysis, the A60-Micro-PLUS was calibrated using a reference bead mix. Briefly, polystyrene and silica beads with diameters ranging from 110 to 1,300 nm (Apogee bead mix #1493) were used to evaluate A60-Micro-PLUS sensitivity for light-scatter detection (FIG. 5). Light-scatter triggering thresholds were set such that all events falling between 110 and 800 nm were gated as EVs. Non-specific fluorescent backgrounds produced by plasmas incubated with isotype controls were used to gate on antibody-positive EVs. Samples were run in duplicates at a flow rate of 1.5 L/minute for 1 minute, resulting in an event rate below 10,000 events per second to avoid coincident particle detection and swarm effect. Quantification of total particles and marker-positive EVs was performed using FlowJo v10 software (FlowJo, Ashland, OR, USA). Particles (including EVs) were gated on large-angle light scattering (LALS) and small-angle light scattering (SALS), and then EV subpopulations were gated on LALS (x axis) and fluorescence intensity (y axis). For sample detection, laser powers were set at 70 mW (405-nm laser), 53 mW (488-nm laser), and 43 mW (638-nm laser). Photomultiplier tube detector voltages for LALS and SALS were set at 300 and 320, respectively. Triggering thresholds for LALS and SALS were set at 20 and 25 (arbitrary units). This methodology was specifically used for the experiments reported in FIG. 1. FIG. 5 represents the gating strategy used for nanoscale flow cytometry of EVs.

To assess the sensitivity of nanoscale flow cytometry for detection and enumeration of PD-L1⁺ EVs, 786-O kidney cancer cells were stably transduced with a lentiviral construct expressing PD-L1 tagged with the fluorescent reporter GFP in C terminus (Origene, Rockville, MD, USA). After sorting of PD-L1-GFP-overexpressing cells, cells were incubated in culture medium supplemented with exosome-depleted FBS (Gibco, Gaithersburg, MD, USA) for 48 hours. Culture medium was collected and centrifuged at 2,500×g for 15 minutes to remove dead cells and debris. PD-L1-GFP⁺ EVs were concentrated using ultrafiltration centrifugal columns with a cutoff of 100 kDa and following the manufacturer's instructions (Amicon, Miami, FL, USA). Several dilutions of PD-L1-GFP⁺ EVs were spiked-in PPP of three normal donors followed by incubation with PD-L1 antibodies or antibody-matched isotype. After analysis by nanoscale flow cytometry, PD-L1-GFP⁺ EVs and PD-L1⁺ EVs detected by anti-PD-L1 were quantified and compared. EVs isolated from 786-0 cells genetically knocked out for PD-L1 expression by CRISPR-Cas9 technology were used as negative controls for PD-L1 staining.

### EV Capture Assay

To estimate EV uptake by T cells, an EV capture assay was performed. EVs were thawed at 37° C. and concentrated to 2×10⁶ EVs/μL. The concentration was measured using nanoscale flow cytometry. 200,000 untransduced (UTD) T cells or CART19 cells were cultured with 20×10⁶ EVs per well in a 96-well plate, with a replicate for each collection time point (0, 2, 4, and 6 hours). At the time of collection, the sample was centrifuged at 300×g for 5 minutes to pellet CART19 and UTD T cells. Supernatants were collected and centrifuged at 2,000×g for 10 minutes to remove cellular debris and aggregates. Supernatants were analyzed by nanoscale flow cytometry. This methodology was specifically used for the experiments reported in FIG. 9.

### EV Characterization by Western Blot

To characterize plasma EVs isolated from CLL patient blood (Table 1), the recommendations provided by ISEV were followed (Théry et al., J. Extracell. Vesicles. 7:1535750 (2018)). Five hundred microliters of PPP was centrifuged at high speed using a Beckman Coulter Optima XPN ultracentrifuge equipped with a Beckman Coulter SW55-Ti rotor. Samples were centrifuged at 100,000×g for 3 hours at 4° C., washed with PBS, and centrifuged again following the same conditions. Pellets were resuspended in 100 μL of radioimmunoprecipitation assay (RIPA) buffer, and protein concentration was measured by a bicinchoninic acid (BCA) protein assay (23225, Thermo Fisher Scientific, Waltham, MA, USA). Thirty micrograms of protein lysates was used for SDS-PAGE electrophoresis. Following transfer, nitrocellulose membranes were blocked with 5% BSA in Tris-buffered saline with Tween 20 (TBST) for 1 hour at room temperature. Membranes were incubated overnight at 4° C. with the following antibodies: rabbit PD-L1 (E1L3N) XP (13684, Cell Signaling Technology, Danvers, MA, USA) (dilution 1:1,000), rabbit CD81 (H-121) (sc-9158, Abcam, Cambridge, MA, USA) (dilution 1:1,000), rabbit CD9 (EPR2949) (ab195422, Abcam, Cambridge, MA, USA) (dilution 1:1,000), and rabbit TSG101 (EPR7130 (B)) (ab125011, Abcam, Cambridge, MA, USA) (dilution 1:1,000). Membranes were washed with TBST and incubated with horseradish peroxidase (HRP)-conjugated secondary antibodies at a dilution of 1:10,000 for 1 hour at room temperature followed by revelation using the SuperSignal West Pico Plus chemiluminescence substrate (Thermo Fisher Scientific, Waltham, MA, USA).

### Generation of CART19 Cells and E-Cadherin-Directed CART Cells

T cells from normal donors were transduced with a replication-incompetent lentiviral vector expressing a second-generation CAR consisting of an anti-CD19 scFv (FMC63) fused to 4-1BB and CD3z intracellular domains as described elsewhere (Sterner et al., Blood, 133:697-709 (2018)) or encoding a second-generation anti-E-cadherin (clone SC10.178) fused to CD28 and CD3z intracellular domains.

### Cell Lines

The mantle cell lymphoma cell line JeKo-1 was purchased from ATCC (CRL-3006, Manassas, VA, USA). For in vivo experiments, JeKo-1 cells were transduced with a luciferase-ZsGreen lentivirus (Addgene, Cambridge, MA, USA) and sorted to 100% purity. JeKo-1 and JeKo-1 Luc-ZsGreen tested negative for mycoplasma (IDEXX, Columbia, MO, USA). The MCF-7 cell line tested negative for mycoplasma (IDEXX, Columbia, MO, USA). Cell lines were cultured in R20 made with RPMI 1640 (Gibco, Gaithersburg, MD, USA), 20% FBS (Corning Life Sciences, Corning, NY, USA), and 1% penicillin-streptomycin-glutamine (Gibco, Gaithersburg, MD, USA). Fresh aliquots of cell lines were thawed at least every 8 weeks (used approximately between passages 2 and 20).

### T Cell Functional Assays

CART19 and JeKo-1 or JeKo-1 cells irradiated at 120 Gy were cocultured at a 1:1 ratio with or without CLL-derived EVs. T cells for functional assays were cultured in T cell medium containing X-VIVO 15 (Lonza, Walkersville, MD, USA), 10% human serum albumin (Innovative Research, Novi, MI, USA), and 1% penicillin-streptomycin-glutamine (Gibco, Gaithersburg, MD, USA). EVs were cocultured with CART19 cells at 100:1, 10:1, 5:1, and 1:1 EV/CART cell ratios using three biological replicates of CLL-derived EVs at 37° C., 5% CO₂, and then co-cultured with primary CLL cells or JeKo-1 cells as indicated in the specific experiment. Cell supernatant was collected at 24 hours, and cells were analyzed by flow cytometry. To assess killing and proliferation, UTD T cells, CART19 cells, and CART19 cells co-cultured with CLL-derived EVs at a 100:1 EV/CART cell ratio were incubated at 37° C., 5% CO₂ for 6 hours before adding JeKo-1 target cells. To assess proliferation with PD-L1 blockade, CART19 cells were co-cultured with and without CLL-derived EVs at a 100:1 EV/CART cell ratio with and without anti-PD-L1 antibody (atezolizumab, 20 μg/mL) at 37° C., 5% CO₂ for 6 hours before adding JeKo-1 target cells. Cells were analyzed by flow cytometry after 48 hours of incubation.

### Flow Cytometric Analysis

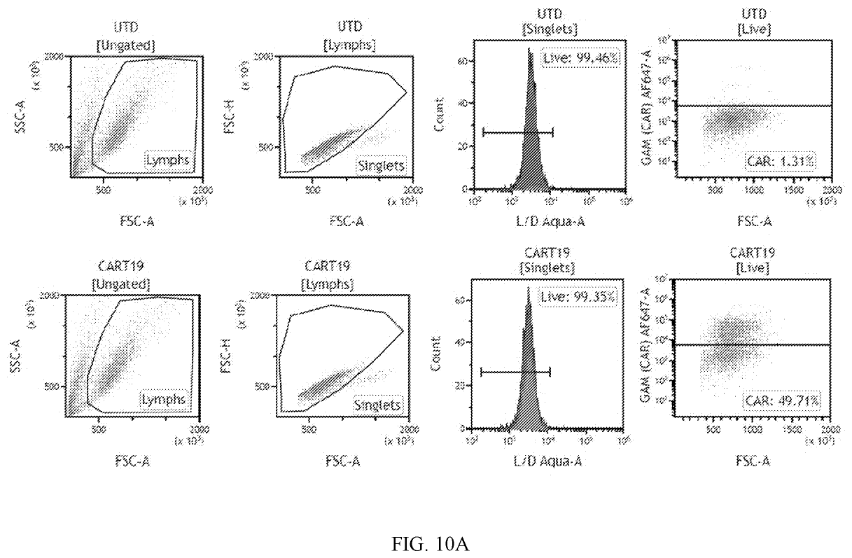

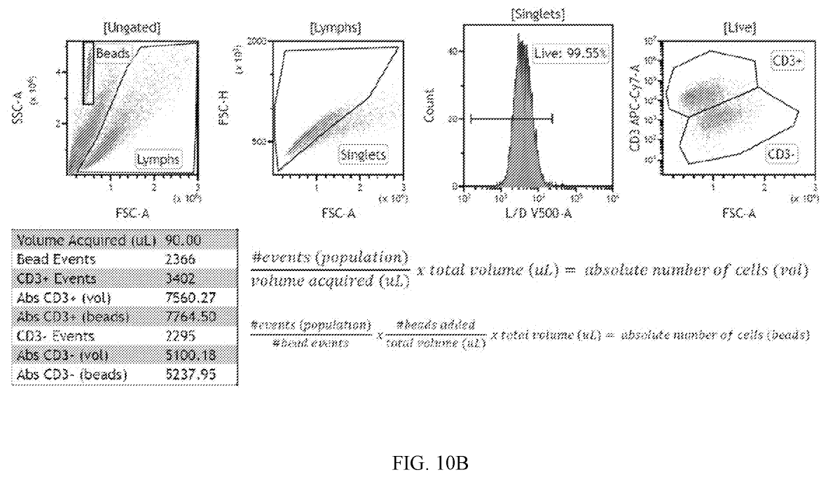

Extracellular staining was performed by washing cells with flow buffer (PBS, 2% fetal bovine serum (FBS) (v/v), and 1% sodium azide (v/v)) and staining with antibodies for 15 minutes. Cells were washed again with flow buffer, and cytometric data were acquired using a CytoFLEX flow cytometer (Beckman Coulter, Chaska, MN, USA). Gating was performed using Kaluza version 2.1 (Beckman Coulter, Chaska, MN, USA). Cells were gated by singlet discrimination, and live cells were determined by Live/Dead Aqua staining (L34966, Thermo Fisher Scientific, Waltham, MA, USA). Surface expression of CAR was detected by staining with a goat anti-mouse F(ab′) 2 antibody (A21235, Invitrogen, Carlsbad, CA, USA). The following antibodies were used: CD279 (clone EH12.2H7) Brilliant Violet 421 (BV421) (329920, BioLegend, San Diego, CA, USA), CD366 (clone F38-2E2) phycoerythrin (PE) (345006, BioLegend, San Diego, CA, USA), CD223 (clone 3DS223H) fluorescein isothiocyanate (FITC) (11-2239-42, eBioscience, San Diego, CA, USA), CD152 (BNI3) PE-Cy7 (369614, BioLegend, San Diego, CA, USA), and CD3 (clone SK7) allophycocyanin (APC)-H7 (560176, BD Pharmingen, San Diego, CA, USA). Absolute quantification was obtained using volumetric measurement. FIG. 10 represents the gating and quantification strategy used for flow cytometric analysis of T cells.

### RNA Isolation

CART19 and irradiated JeKo-1 cells were co-cultured at a 1:1 ratio for 24 hours with CLL-derived EVs at 10:1 and 1:1 EV/CART19 cell ratios. Three biological replicates of CLL-derived EVs were included as well as stimulated and unstimulated CART19 cell controls. CART19 cells were isolated using magnetic sorting with CD4 and CD8 microbeads (catalog nos. 130-045-101 and 130-045-201, Miltenyi Biotec, Auburn, CA, USA). RNA was isolated from the CART19 cells using a QIAGEN miRNeasy micro kit (217084, QIAGEN, Germantown, MD, USA). To account for donor-donor variability, RNA-seq was performed on CART19 cells generated from a specific donor and cultured with EVs derived from multiple CLL patients.

### Rna-Seq and Analysis

Total RNA was prepared with a SMARTer stranded total RNA-seq kit v2, Pico input mammalian (Takara, Mountain View, CA, USA). Total RNA (three samples per lane) was sequenced on an Illumina HiSeq 4000 (Illumina, San Diego, CA, USA). Fastq files were viewed in FastQC v0.11.8 to check for quality. Adaptor sequences were removed using Cutadapt v1.18. Output files were re-checked for quality and adaptor removal using FastQC v0.11.8. Raw sequencing data are available at the Gene Expression Omnibus (GEO: GSE147046).

The latest human reference genome (GRCh38) was downloaded from NCBI. Genome index files were generated using STAR v2.5.4b. Paired end reads from the trimmed fastq files were mapped to the genome. HTSeq (Python 3.6.5) was used to generate expression counts for each gene. DESeq2 (R v3.6.1, R-project.org/) was used to normalize gene counts (geometric mean) and calculate differential expression using adjusted p values <0.05. A heatmap was created using pheatmap (cran.r-project.org/web/packages/pheatmap/index.html). Networks were generated using Ingenuity Pathway Analysis v49932394 (QIAGEN, qiagenbioinformatics.com/products/ingenuity-pathway-analysis). Gene set enrichment analyses were performed using Enrichr (maayanlab.cloud/Enrichr/).

### In Vivo Mouse Experiments

6- to 8-week-old non-obese diabetic (NOD)-severe combined immunodeficiency (SCID)-interleukin (IL)-2rγ^{-/-} (NSG) mice were purchased from Jackson Laboratory (Bar Harbor, ME, USA) and injected intravenously with 1×10⁶ cells from the JeKo-1 Luc-ZsGreen mantle cell lymphoma cell line. Upon engraftment, mice were randomized to receive either (1) UTD T cells, (2) CART19 cells, or (3) CART19 cells co-cultured ex vivo with CLL-derived EVs for 6 hours (100:1 EV/CART cell ratio). All conditions were co-cultured for 6 hours, washed, and injected at a dose of 2.5×10⁵ cells intravenously. Mice were followed with serial bioluminescence imaging to measure tumor burden.

### Statistical Analyses

All statistics were performed using GraphPad Prism version 7.05 for Windows (GraphPad, La Jolla, CA, USA) or DESeq2. Statistical tests are described in detail in the figure legends. Briefly, a Mann-Whitney test was used to test the hypotheses for EV immunophenotype. One-way ANOVA was used to test the hypotheses for inhibitory receptor expression, proliferation, and killing. Two-way ANOVA was used to test the hypotheses for in vivo tumor burden, and a log-rank test was used to test the hypotheses for in vivo survival. mRNA differential expression multiple hypothesis correction was performed using Benjamini-Hochberg procedure within DESeq2.

### Example 2: PD-L1^{high} EV Populations as a Marker of CART Cell Therapy Responsiveness

Experiments described in this Example were performed as described in Example 1 and/or in Example 3.

### Results

The Presence of a PD-L1^{high} EV Population is Associated with Lack of Response in Patients with Lymphoma Treated with CART19 Cell Therapy

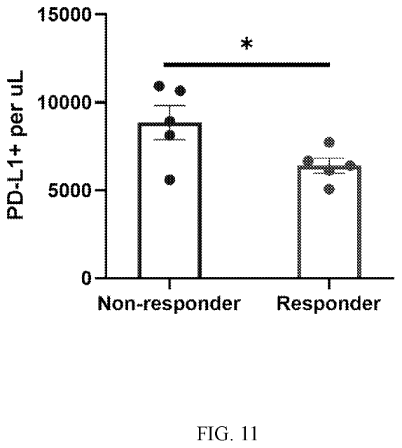

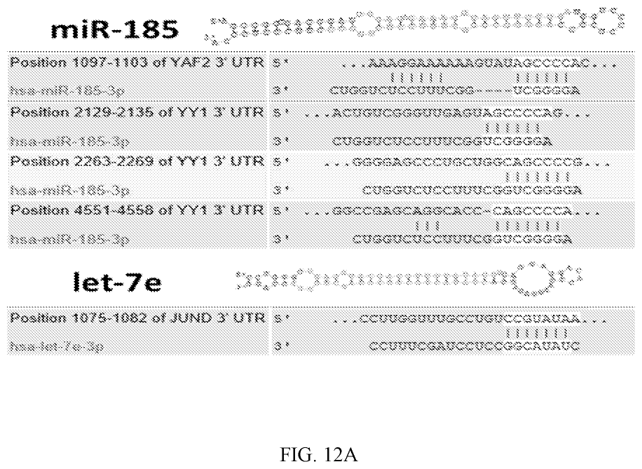

PD-L1⁺ EVs were enumerated from platelet-free plasma of baseline samples using nanoscale flow cytometry. CART19 cell therapy non-responders exhibit significantly more PD-L1+ EVs compared to responders prior to treatment (FIG. 11). These results demonstrate that baseline PD-L1⁺ EV levels in sample that are readily obtainable in a non-invasive manner can be may be used a biomarker to predict response to CART19 cell therapy.

### MicroRNAs Targeting T Cell Activation Pathways are Altered in CART Cells

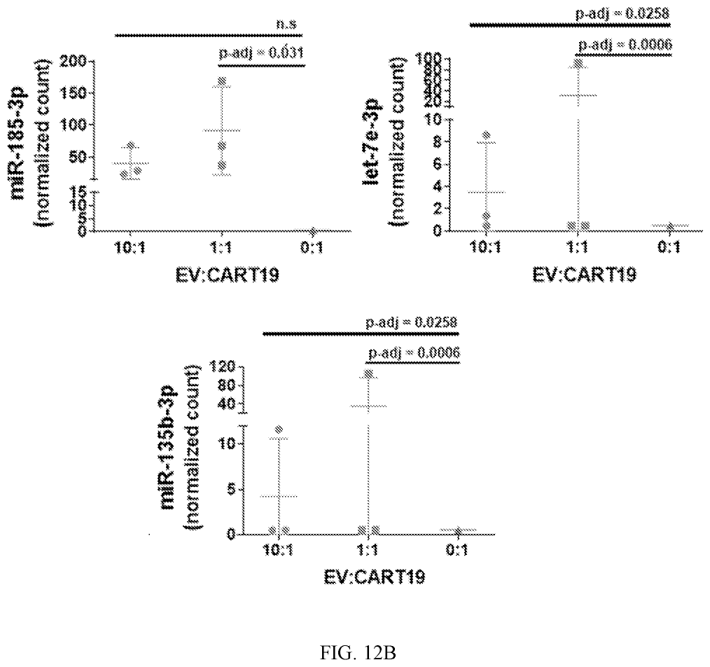

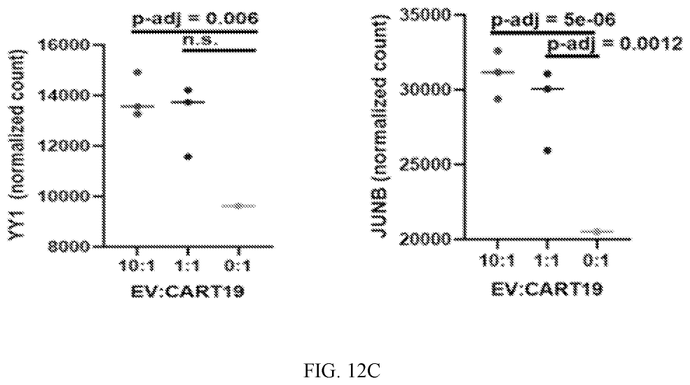

miRNA are significantly upregulated in antigen-activated CART19 cells co-cultured with CLL-derived EVs. TargetScan was to predict that miR-185 and let-7e target AP-1-associated genes YY1, JUND, and YAF2 (FIG. 12A). miR-185-3p, let-7e-3p, and miR-135b-3p were significantly upregulated in antigen-activated CART19 cells when co-cultured with CLL-derived EVs at a 1:1 or 10:1 EV:CART19 cell ratio (FIG. 12B). YY1 and JUNB expression was significantly upregulated in antigen-activated CART19 cells when co-cultured with CLL-derived EVs (FIG. 12C). These results demonstrate that the miRNA cargo in the EVs can alter the miRNA signature and gene expression of the CART19 cells, altering exhaustion pathways.

miR-185-3p Mimic Inhibits CART Cell Killing

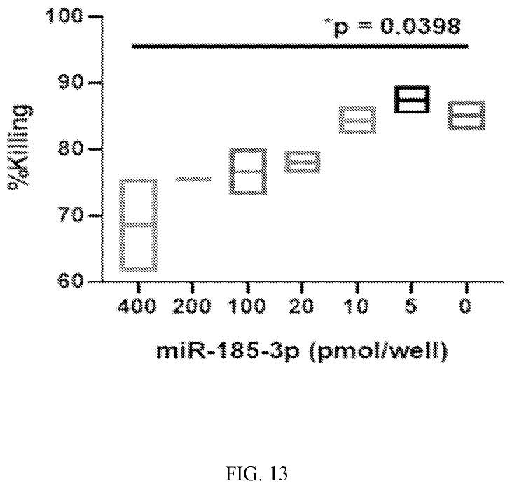

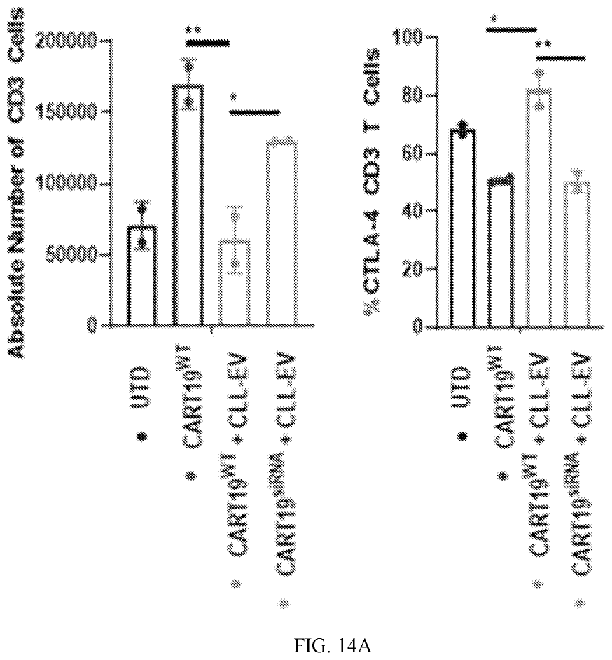

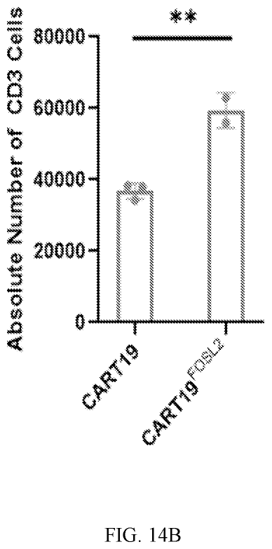

miR-185-3p mimic inhibits CART19 cell killing at high doses (FIG. 13). These results demonstrate that miR-185-3p can impact CART19 cells independent of extracellular vesicles.

### Example 3: PD-L1^{high} EV Populations as a Marker of CART Cell Therapy Responsiveness

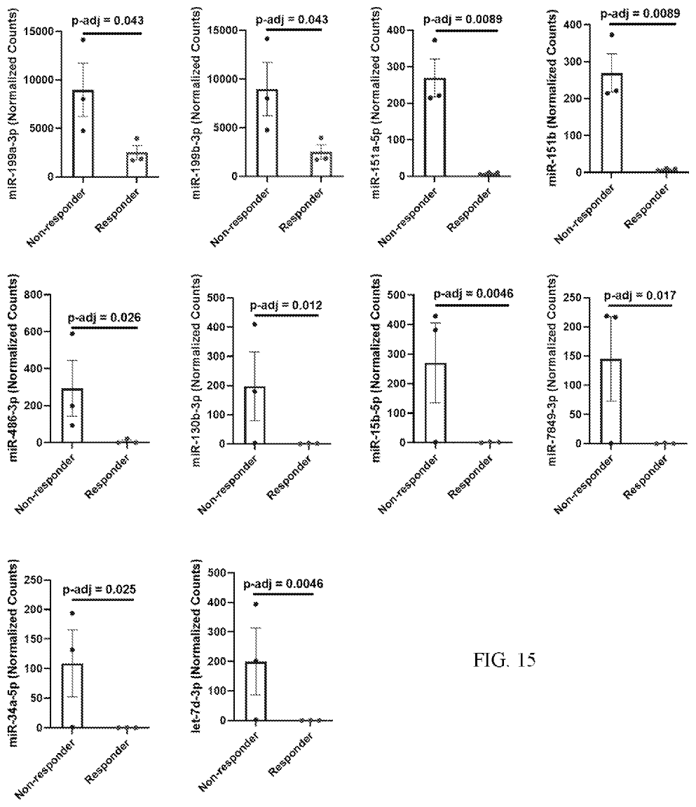

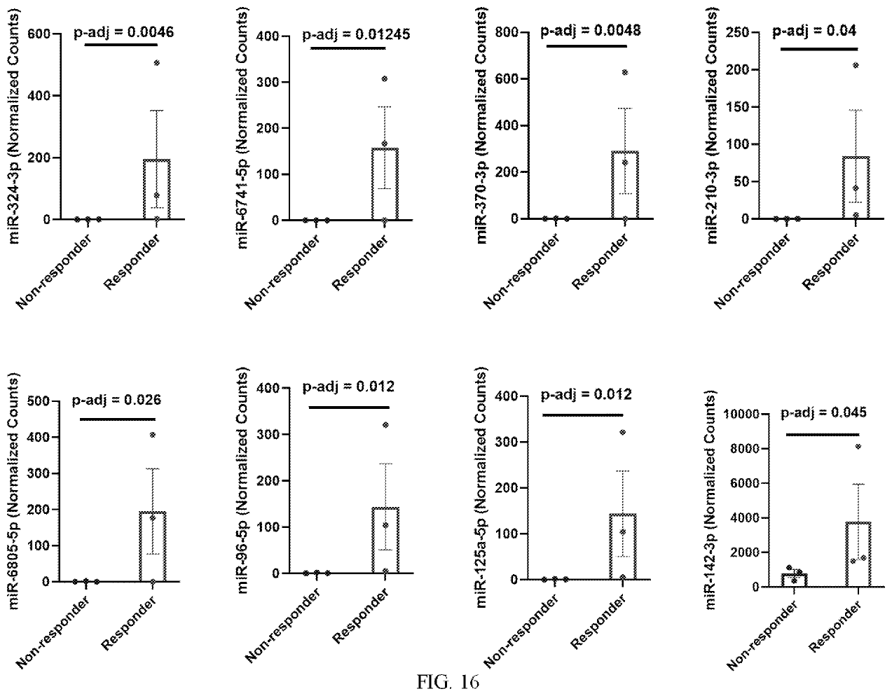

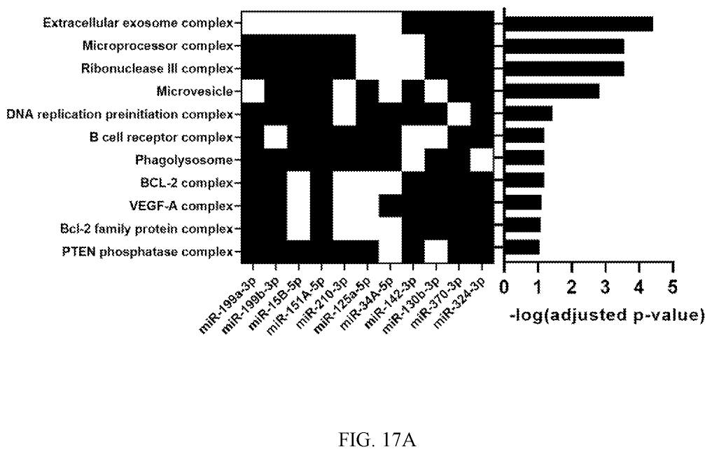

EVs in PD-L1^{high} EV populations were examined for their cargo content. A distinct microRNA signature was identified in EVs from PD-L1^{high} EV populations from cancer patients that did not respond to CART19 cell therapy (non-responders) as compared to the microRNA signature of EVs from PD-L1^{low} EV populations in patients that did respond to CART19 cell therapy (responders). Ten microRNAs (miR-199a-3p, miR-199b-3p, miR-151a-5p, miR151b, miR-486-3p, miR-130b-3p, miR15b-5p, miR-7849-3p, miR-34a-5p, and let-7d-3p) were upregulated in non-responders (FIG. 15). Eight microRNAs (miR-324-3p, miR-6741-5p, miR370-3p, miR-210-3p, miR-6805-5p, miR96-5p, miR-125a-5p, and miR142-3p) were downregulated in non-responders (FIG. 16). Table 2 shows gene targets and functions of miRNAs as indicated by Ingenuity Pathway Analysis.

TABLE 2

| | Adj | | | |
|---|---|---|---|---|
| | p- | Expression | | |
| miRNA | value | (NR) | Gene Targets | Function |
| miR- | 0.04 | DOWN | PTPN1 | JAK/Stat Signaling, Protein Kinase A Signaling |
| 210-3p | | | | |
| miR- | 0.045 | DOWN | PRKCA | aApoptosis Signaling |
| 142-3p | | | | |
| miR- | 0.043 | UP | RUNX1; CD44; MET; | AML signaling; GM-CSF signaling; IL-23 signaling; |
| 199b- | | | MTOR; PTGS2 | leukocyte extravasion signaling; tumor microenvironment |
| 3p | | | | pathway; IL-15signaling; IL-7 signaling; IL-4 signaling; |
| | | | | IL-8 signaling; T cell receptor signaling; |
| | | | | T cell exhaustion signaling |
| miR- | 0.0046 | UP | ASXL2; ATF6; BCL2; | senescence pathway; PI3K signaling in B lympthocytes; |
| 15b-5p | | | BCL2L2; CCND1; CCND3; | apoptosis signaling; tumor microenvironment pathway; |
| | | | CCNE1; CDC25A; E2F3; | STAT3 pathway; VEGF signaling; IL-6 signaling; IL-4 |
| | | | EGFR; FGF2; FDF7; | signaling; IL-7 signaling; IL-8 signaling; CM-CSF signaling; |
| | | | FGFR1; FLT3; GRB2; | PTEN signaling; CML signaling; B cell receptor signaling; |
| | | | HMGA1; HSP90B1; IFNG; | NFKB signaling; MAPK signaling; T cell exhaustion signaling |
| | | | ITGA2; JUN; JUNB; | pathway; T helper differentiation; Th1 and Th2 activation |
| | | | KITLG; KPNA3; LAMTOR3; | pathway; Th17 activation pathway; Tec kinase signaling; |
| | | | MAP2K1; MCL1; NOTCH2; | apoptosis signaling; mTOR signaling; IL-17 signaling; |
| | | | PPP2R5C; PTGS2; RAF1; | p53 signaling |
| | | | SERPINE2; SPI1; SQSTM1; | |
| | | | TNFSF9; VEGFA; WT1 | |
| let-7d- | 0.0046 | UP | HIF-1; RAS; ERB | Apoptosis pathways |
| 3p | | | | |
| miR- | 0.025 | UP | BCL2; CCND1; CDK6; CREB1; | STAT3 pathway; VEGF signaling; tumor microenvironment |
| 34a-5p | | | DLL1; E2F3; E2F5; FOXP1; | pathway; CML signaling; ERK/MAPK signaling; Th1 and Th2 |
| | | | HDAC1; JAG1; MAP2K1; MET; | activation pathway; T cell exhaustion signaling pathway; |
| | | | MYC; MYCN; NOTCH2; SIRT1; | calcium-induced T lymphocyte apoptosis; IL-15 production; |
| | | | FP53; VEGFA | IL-7 signaling; AML signaling |

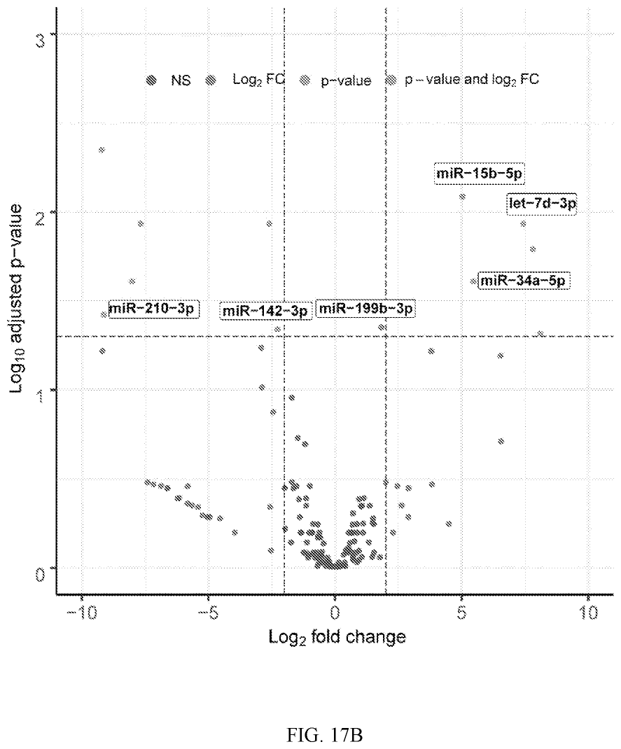

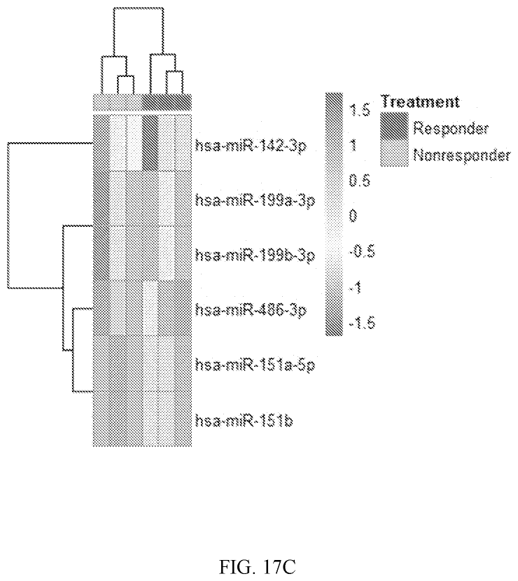

Gene set enrichment analysis was performed using Enrichr. Gene set enrichment analysis shows that the significantly different miRNAs enrich for B cell receptor complex, BCL-2 complex, VEGF-A complex, and PTEN phosphatase complex (FIGS. 17A-17C).

### Materials and Methods

EVs were isolated from baseline platelet-free plasma samples from 3 responders and 3 non-responders CART19-treated lymphoma patients using ExoQuick ULTRA. miRNA was isolated from the purified EVs using miRNeasy Micro Kit. Small RNA library was prepped using QIAseq miRNA Library Kit and sequenced on Illumina HiSeq 4000. Adapter sequences were removed using CutAdapt and analyzed using miRDeep2. DeSeq2 was used to normalize miRNA counts and differential expression. FDR is calculated using Benjamini-Hochberg step-down procedure.

### Example 4: CAR T Editing to Overcome EV-Induced Exhaustion

CAR T cells are engineered to alter expression of one or more genes involved in T cell exhaustion pathways and/or one or more genes involved in other pathways involved in immunotherapy effectiveness. In some cases, a CAR T cell are engineered to alter expression of a gene for which expression is targeted by one or more miRNAs enriched in CART19 cell therapy non-responders. Gene expression can be upregulated or downregulated. Examples of genes that are involved in T cell exhaustion pathways and are targeted by miRNAs that are altered in CART19 cell therapy non-responders are shown in Table 3.

TABLE 3

| | | miRNA | |
|---|---|---|---|
| | miRNA | (UP/DOWN) | |
| Gene to | Targeting | in non- | |
| Alter | the Gene | responders | Pathway(s) Involved |
| FOXO1 | miR-96-5p | DOWN | T cell exhaustion pathways |
| FOXP1 | miR-34a-5p | UP | T cell exhaustion pathways |
| MTOR | miR-199b-3p | UP | T cell exhaustion pathways |
| | | | Senescence pathway |
| PPP2R5C | miR-15b-5p | UP | T cell exhaustion pathways |
| | | | CTLA4 signaling in cytotoxic |
| | | | T lymphocytes |
| | | | Senescence pathway |
| VEGFA | miR-15b-5p | UP | T cell exhaustion pathways |
| | miR-34a-5p | | |
| ACVR1B | miR-210-3p | DOWN | T cell exhaustion pathways |
| | | | Senescence pathways |
| GRB2 | miR-15b-5 | UP | CTLA4 signaling in cytotoxic |
| | | | T lymphocytes |
| | | | Nfat signaling pathways |
| BCL21 | miR-142-3p | DOWN | PD1 signaling pathways |
| IFNG | miR-15b-5p | UP | T cell exhaustion pathways |
| | | | PD1 signaling pathways |
| JUN | miR-15b-5p | UP | Nfat signaling pathways |
| | | | Senescence pathway |
| KPNA3 | miR-15b-5p | UP | Nfat signaling pathways |
| PRKCA | miR-142-3p | DOWN | Nfat signaling pathways |
| | | | Apoptosis signaling |
| HDAC1 | miR-34a-5p | UP | Nfat signaling pathways |
| MAP2K1 | miR-34a-5p | UP | Nfat signaling pathways |
| | | | Senescence pathway |
| MAP2K3 | miR-15b-5p | UP | Nfat signaling pathways |
| | | | Senescence pathway |
| MAP2K7 | miR-125a-5p | DOWN | Nfat signaling pathways |
| | | | Senescence pathway |
| RAF1 | miR-15b-5p | UP | Nfat signaling pathways |
| | | | Senescence pathway |
| SMAD4 | miR-130b-3p | UP | Antiproliferative Role of TOB |
| | | | in T Cell Signaling |
| | | | Regulation of IL-2 Expression |
| | | | in Activated and Anergic T |
| | | | Lymphocytes |
| | | | Senescence pathway |
| BCL2 | miR-34a-5p | UP | Death Receptor Signaling |
| | | | Apoptosis Signaling |
| BCL2L2 | miR-15b-5p | UP | Death Receptor Signaling |
| | | | Apoptosis Signaling |
| CASP6 | miR-125a-5p | DOWN | Death Receptor Signaling |
| | | | Apoptosis Signaling |
| CASP7 | miR-125a-5p | DOWN | Death Receptor Signaling |
| | | | Apoptosis Signaling |
| CCNE1 | miR-15b-5p | UP | Senescence pathway |
| ASXL2 | miR-15b-5p | UP | Senescence pathway |
| CBX7 | miR-125a-5p | DOWN | Senescence pathway |
| CCND1 | miR-34a-5p | UP | Senescence pathway |
| CCND3 | miR-15b-5p | UP | Senescence pathway |
| CCNE1 | miR-15b-5p | UP | Senescence pathway |
| CDC25A | miR-15b-5p | UP | Senescence pathway |
| CDK6 | miR-15b-5p | UP | Senescence pathway |
| | miR-34a-5p | | |
| CDKN2 | miR-125a-5p | DOWN | Senescence pathway |
| DMTF1 | miR-15b-5p | UP | Senescence pathway |
| E2F3 | miR-210-3p | DOWN | Senescence pathway |
| | miR-15b-5p | UP | |
| | miR-34a-5p | UP | |
| E2F5 | miR-34a-5p | UP | Senescence pathway |
| SIRT1 | miR-34a-5p | UP | Senescence pathway |
| SQSTM1 | miR-15b-5p | UP | Senescence pathway |
| TP53 | miR-125a-5p | DOWN | Senescence pathway |
| | miR-34a-5p | UP | |

### Example 5: Combination Treatment with CAR T and Compound(s) to Block EV Trafficking and/or Production

CAR T cells are administered together with one or more agents that inhibit EV production and/or EV trafficking. In some cases, the one or more agents that can inhibit EV production and/or EV trafficking are administered at the same time. In some cases, the one or more agents that can inhibit EV production and/or EV trafficking are administered independently. For example, one or more cancer immunotherapies are administered first, and the one or more agents that can inhibit EV production and/or EV trafficking are administered second, or vice versa.

Examples of agents that can inhibit EV production and/or EV trafficking that can be administered to a mammal (e.g., a human) together with one or more immunotherapies are as described below.

### Calpeptin

Calpeptin is obtained from Selleck Chemicals (Catalog No. S7396) is prepared in ethanol at 50 to 100 mg/mL (e.g., 72 mg/mL; 198.64 mM), and administered to a mammal (e.g., a human) having cancer at a dose of 1 to 100 μM (e.g., 10, 25, or 50 μM). The prepared calpeptide can be stored at −80° C. for up to 2 years prior to being administered.

### Manumycin A

Manumycin A is obtained from Sigma Aldrich (Product No. M6418) is prepared in methanol at 5 to 15 mg/mL (e.g., 10 mg/mL). The prepared nanumycin A can be stored at 4° C. prior to being administered.

### Y27632

Y27632 is obtained from Selleck Chemicals (Catalog No. S1049) is prepared in water at 50 to 100 mg/mL (e.g., 64 mg/mL; 199.83 mM), and administered to a mammal (e.g., a human) having cancer at a dose of 1 to 50 μM (e.g., 10, 20, 25, or 30 μM). The prepared Y27632 can be stored at −80° C. for up to 2 years prior to being administered.

### D-Pantethine

D-pantethine is obtained from Selleck Chemicals (Catalog No. S5220) is prepared in water at 50 to 150 mg/mL (e.g., 100 mg/mL; 180.27 mM). The prepared D-pantethine can be stored at −80° C. for up to 2 years prior to being administered.

### Imipramine

Imipramine is obtained from Selleck Chemicals (Catalog No. S4377) is prepared in water at 50 to 100 mg/mL (e.g., 63 mg/mL; 198.81 mM). The prepared imipramine can be stored at −80° C. for up to 2 years prior to being administered.

### GW4869

GW4869 is obtained from Selleck Chemicals (Catalog No. S7609) is prepared in DMSO at 0.5 to 5 mg/mL (e.g., 1 mg/mL; 1.73 mM), and administered to a mammal (e.g., a human) having cancer at a dose of 5 to 50 μM (e.g., 10 or 20 μM). The prepared GW4869 can be stored at −80° C. for up to 2 years prior to being administered.

### Example 6: CART Cells and Senolytics

### Methods:

### Navitoclax Treatment

CART cell generation is an 8-day process, so experiments were done with D8 CART cells.

D8 CART cells were treated with navitoclax (0 μM, 0.5 μM, 1 μM, 2 μM, or 4 μM) for 24 hours and then divided into two groups. In the first group, navitoclax was washed away for proliferation and cytotoxicity assays. In the second group, navitoclax treatment was maintained during proliferation and cytotoxicity assays.

Longitudinal navitoclax treatment of CART cells. D15 and D22 CART cells were generated to mimic possible dysfunctions (e.g., exhaustion, senescence, etc.) that CART cells can develop. Serial activation of CART cells can induce T cell senescence during CART cell generation. This experiment was designed to selectively remove senescing CART cells during CART cell proliferation. During this experiment CART cells were pretreated with navitoclax, the proliferation, EDU, and cytotoxicity assays were done in the absence of navitoclax. Longitudinal treatments included two parts. Part I was a typical CART cell production protocol except for addition of navitoclax at D7 for 24 hours. Briefly, CART cells were activated on day 1, transduced on day 2, and debeaded on day 6. On day 7, CART cells were treated with navitoclax (0 μM, 0.125 μM, 0.25 μM, 0.5 μM, 0.75 μM, 1 μM, or 2 μM). On day 8, the navitoclax was washed away, and the CART cells were prepared for cytotoxicity assays and proliferation assays. For part II, JeKo-1 UDT was added to the CART cells on days 8 and 10, the media was replaced on day 12, and navitoclax was added on day 13. On day 14, the navitoclax was washed away, and the CART cells were prepared for cytotoxicity assays and proliferation assays or JeKo-1 IR was added and the CART cells were prepared for an EdU assay. The CART cells were provided several resting times after being activated by JeKo-1 to provide plenty of resting time to differentiate reversible exhaustion and irreversible T cell senescence. This assay focuses on D8 (freshly generated CART cells), D15 (D8 cells activated with JeKo-1 for a week as indicated as part II in the figure), and D22 CART cells (additional activation summarized in part II on D15 CART cells).

### Proliferation Assays

For proliferation assays, effector cells (CART cells), and irradiated target cells (CD19+ JeKo 1 cells) were co-cultured at different ratios and with different concentrations of the compounds. Cells were co-cultured for 3-5 days, and then cells were harvested and surface staining with antihCD3 (eBioscience, San Diego, CA, USA) and LIVE/DEAD™ Fixable Aqua Dead Cell Stain Kit (Invitrogen, Carlsbad, CA, USA) was performed.

### Cytotoxicity Assays

For killing assays, the CD19+ Luciferase+ mantle cell lymphoma cell like JeKo-1 cells were incubated with effector T cells for 24, 48, or 72 hours. Killing was calculated by bioluminescence imaging on a Xenogen IVIS-200 Spectrum camera (PerkinElmer, Hopkinton, MA, USA) as a measure of residual live cells. Samples were treated with 1 μL D-luciferin (30 μg/mL) per 100 μL sample volume (Gold Biotechnology, St. Louis, MO, USA), for 10 minutes prior to imaging.

### EdU Assays

Effector cells (CART cells) were labeled with EdU and then co-cultured with irradiated target cells (CD19+ JeKo 1 cells) at different ratios and with different concentrations of the compounds. Cells were co-cultured for 3-5 days, and then cells were harvested and surface staining with antihCD3 (eBioscience, San Diego, CA, USA) and LIVE/DEAD™ Fixable Aqua Dead Cell Stain Kit (Invitrogen, Carlsbad, CA, USA) was performed.

### In Vivo Assays

NSG mice were engrafted with the luciferase positive, CD19+ JeKo1 cell line I.V. One week later, mice underwent bioluminescence imaging (BLI) to determine the level of the disease and then randomized to treatment with CART cells. Mice then underwent weekly BLI to measure disease burden and were followed for survival.

### Results:

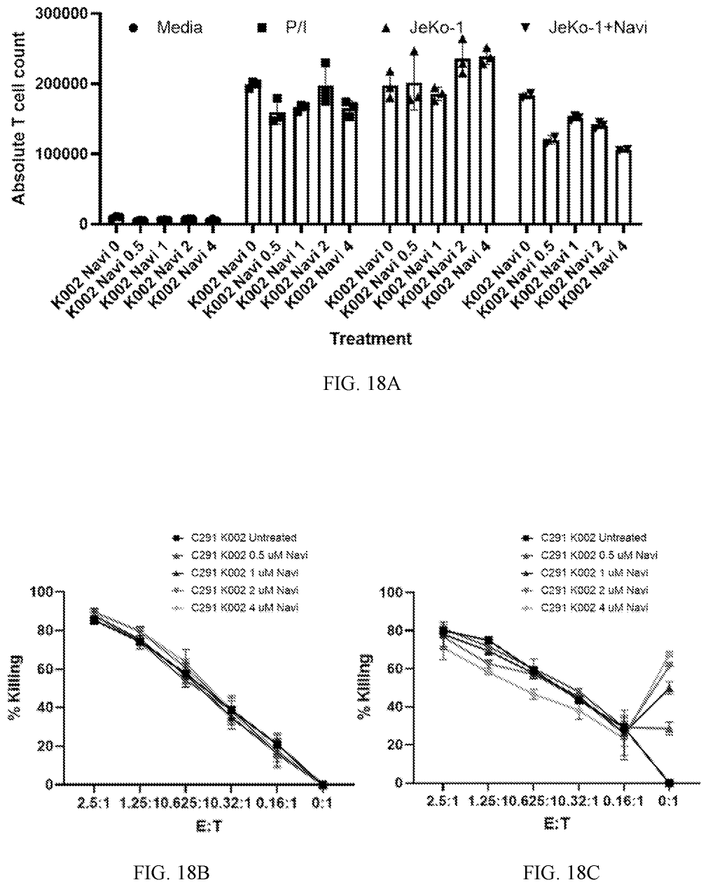

To test the effect of navitoclax on CAR19 T cell cytotoxicity against CD19⁺ mantle cell lymphoma cell line JeKo-1, D8 CART cells were treated with navitoclax for 24 hours. CAR19 T cells with 4-1BB costimulatory domain were combined with senolytic navitoclax (BCL-2 inhibitor). CART cells were also cultured in the presence of JeKo-1 cells with or without navitoclax. The number of CART cells was determined by flow analysis and plotted. CART cells continuously treated with navitoclax resulted in decreased proliferation compared CART cells that were not treated with navitoclax (FIG. 18A). The navitoclax pre-treatment of CART cell did not result in different cytotoxicity (FIG. 18B). Navitoclax alone was cytotoxic against JeKo-1 with 0:1 (E:T means no CART, it is just JeKo-1 with navitoclax) ratio, and decreased CART cell cytotoxicity was observed in higher navitoclax concentrations (FIG. 18C).

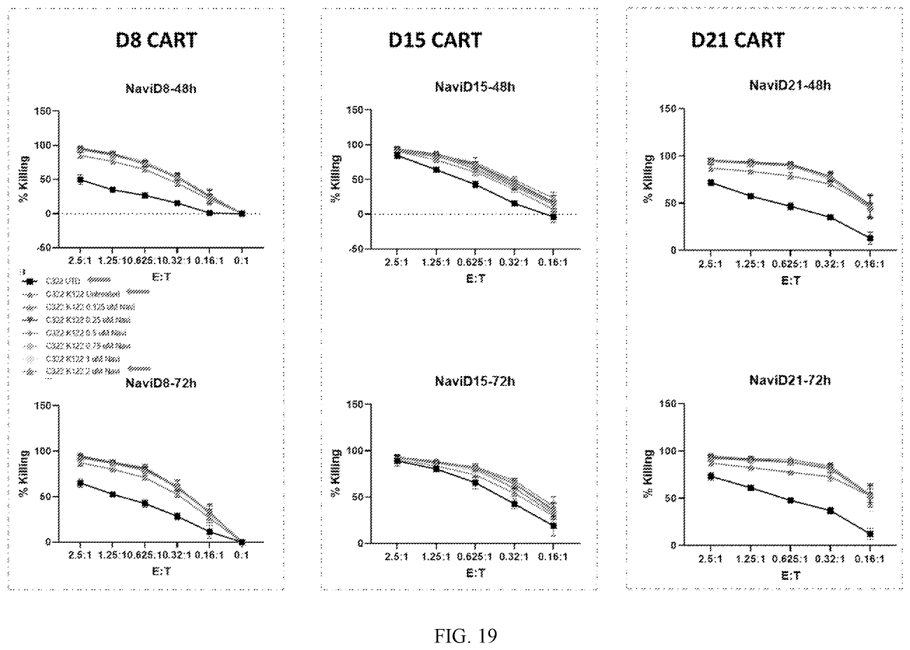

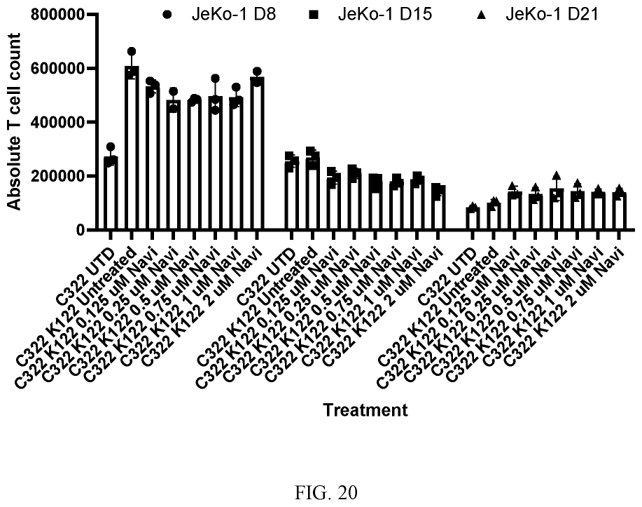

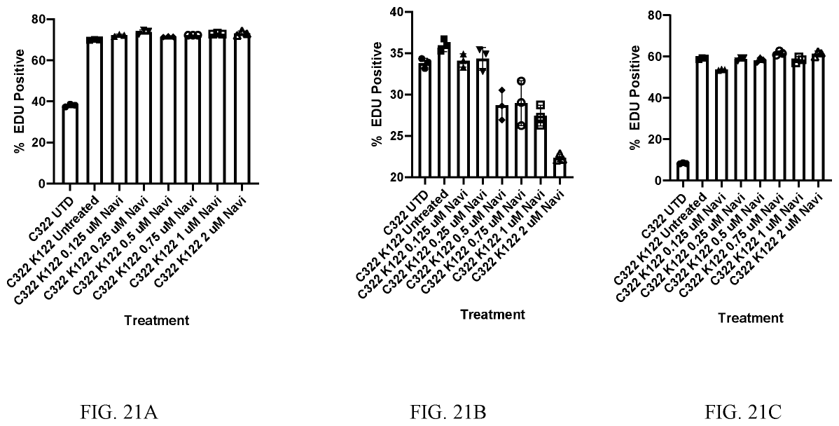

To remove senescing CART cells during CART cell proliferation, longitudinal navitoclax treatment of CART cells was used. D8, D15, and D22 CART cells were prepared using longitudinal navitoclax treatment. Each population of CART cells was co-cultured with varying E:T JeKo-1 ratio. The viability of JeKo-1 was measured, and, as an indication of CART cell cytotoxicity, the percent JeKo-1 death was plotted at 24 hours and 48 hours after the CART-JeKo-1 co culture. The cytotoxicity assays showed an increase in CART cell activity compared to untreated CART cells (FIG. 19). Proliferation of CART cells was measured by counting T cell in the CART-JeKo-1 co-culture by flowcytometer. The proliferation of D8 CART cells varied at all time points tested. Longitudinal navitoclax treatment of CART cells decreased proliferation of CART cells through time, with an increase in D22 CART proliferation in navitoclax treated groups compared to navitoclax untreated CART cell in vitro. (FIG. 20). EdU assays were used to measure the percentage of cycling CART cells treated with navitoclax. D8, D15, and D22 CART cells were co-cultured with JeKo-1 cells in the presence of EdU, a base analog that is incorporated into the genome of cycling cells. Senescing cells have decreased cell cycle, and therefore have decreased EdU. EdU positivity was similar at D8 (FIG. 21A) and D22 (FIG. 21C) CART cells, and was decreased in navitoclax treated CART cells at D15 (FIG. 21B).

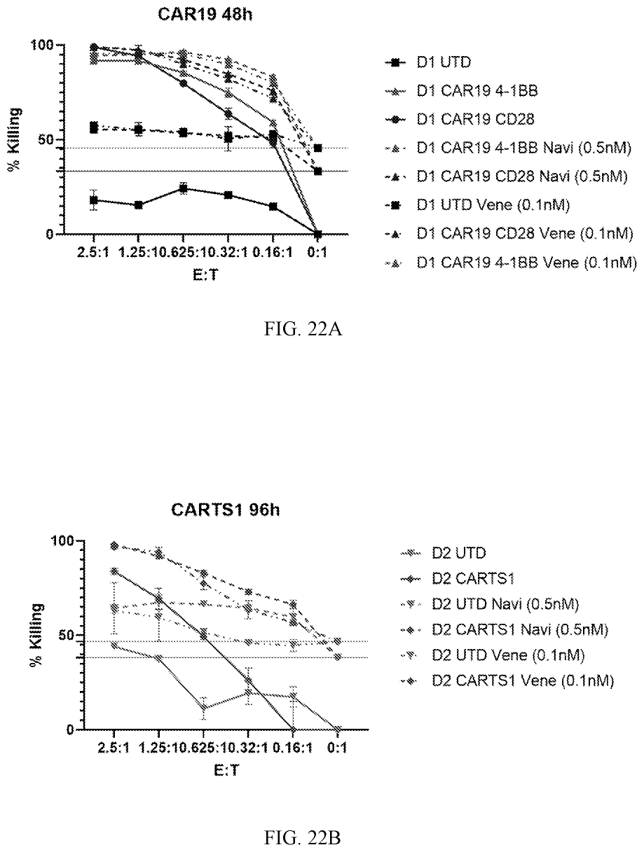

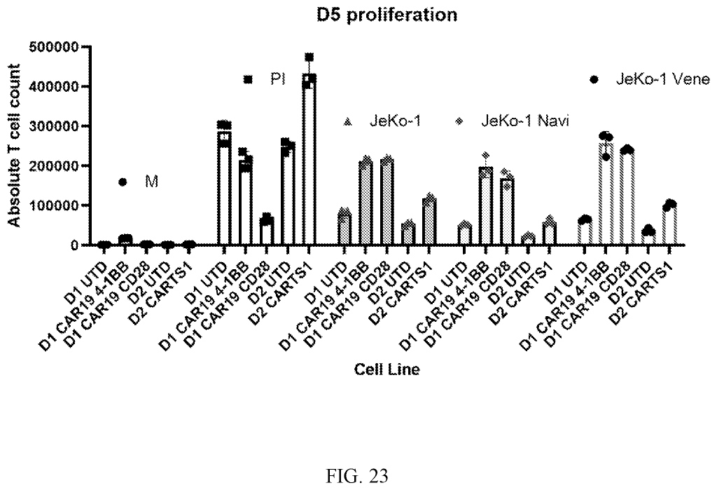

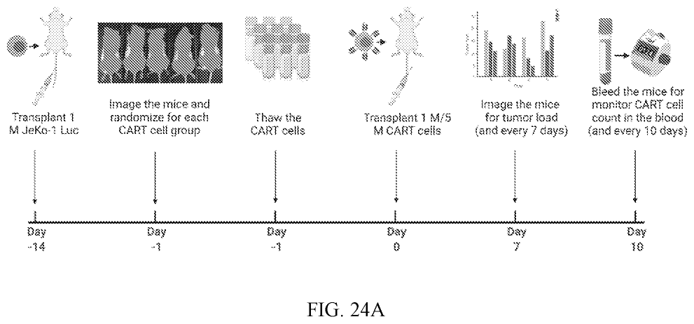

To determine whether decreased proliferation in CART cells continuously treated with navitoclax could be due to affinity of navitoclax with BCL-xl instead of BCL-2 in CART cells in activated CART cells, venetoclax, whose affinity to BCL-xl is less than navitoclax, was used to treat CART cells. D8 CART cells were co-cultured with JeKo-1 cells alone or with JeKo-1 cells and either navitoclax or venetoclax. The survival of the JeKo-1 cells was measured 48 hours after the beginning of co-culture. Both venetoclax and navitoclax combination therapies with CART cells resulted in increased CART cell cytotoxicity against JeKo-1 cell line (FIGS. 22A and 22B). These results demonstrate that supplementing CART cells with senolytics can increase cytotoxicity of the CART cells. CART cells combined with navitoclax demonstrated decreased CART cell proliferation against JeKo-1 cell line while combination with venetoclax (a BCL-2 inhibitor) did not lead to decreased proliferation (FIG. 23), suggesting CART cells and venetoclax can be safely combined.

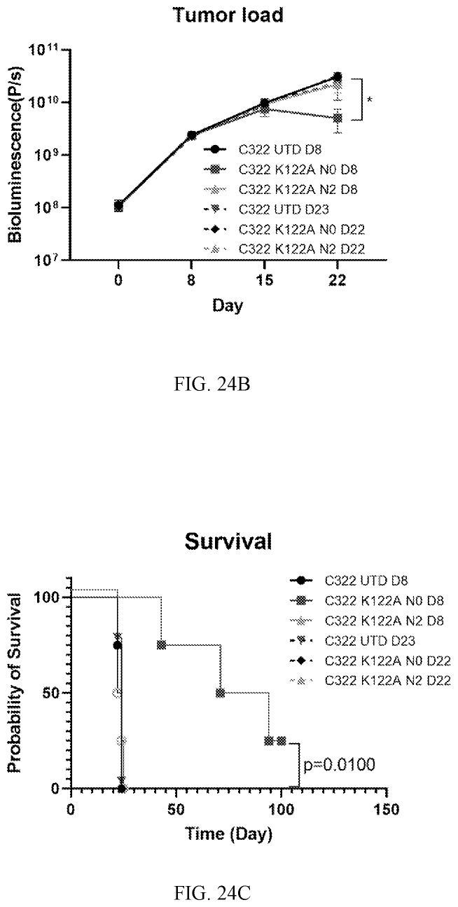

To evaluate the combinatorial effects of CART cells and senolytics in vivo, D8 and D22 CART cells were used in an in vivo model. The tumor burden of the mice at D22 (22 days after CART cell transplantation to the mice) was not different D22 CART cells that were treated with or without navitoclax (FIG. 24B). The only CART cell that still showed antitumor activity was D8 CART cells that had not been treated with navitoclax (FIG. 24C). These results suggested that repeated activation led to decreased CART cytotoxicity such that navitoclax pretreatment did not increase antitumor activity of CART cells in vivo.

### Example 7: Tumor EVs and miRNA as a Biomarker of Response to CART Cell Therapy and Development of Exhaustion Resistant CART

### Methods:

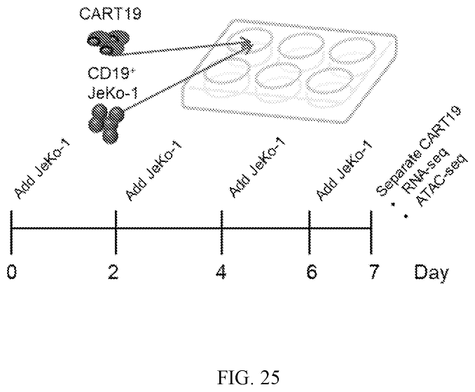

A schematic of an in vitro model for CART exhaustion is shown in FIG. 25.

CART19 cells generated from normal donors were co-cultured with CD19+ JeKo-1 cells at 1:1 ratio to specifically stimulate CART cells through the CAR receptor. JeKo-1 cells were added every 2 days to induce repeated stimulation of CART cells. CART cells were harvested at the end of culture, tested for effector functions, and analyzed for RNA sequencing and ATAC sequencing.

### Results:

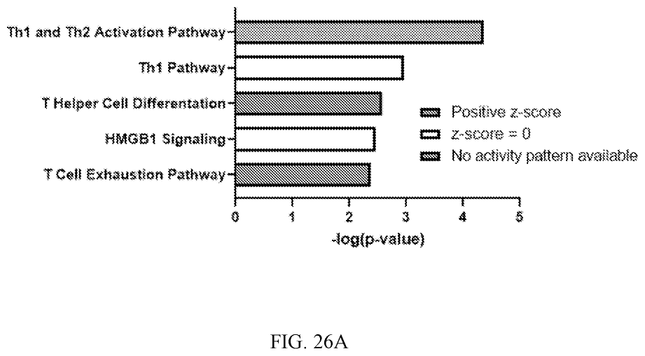

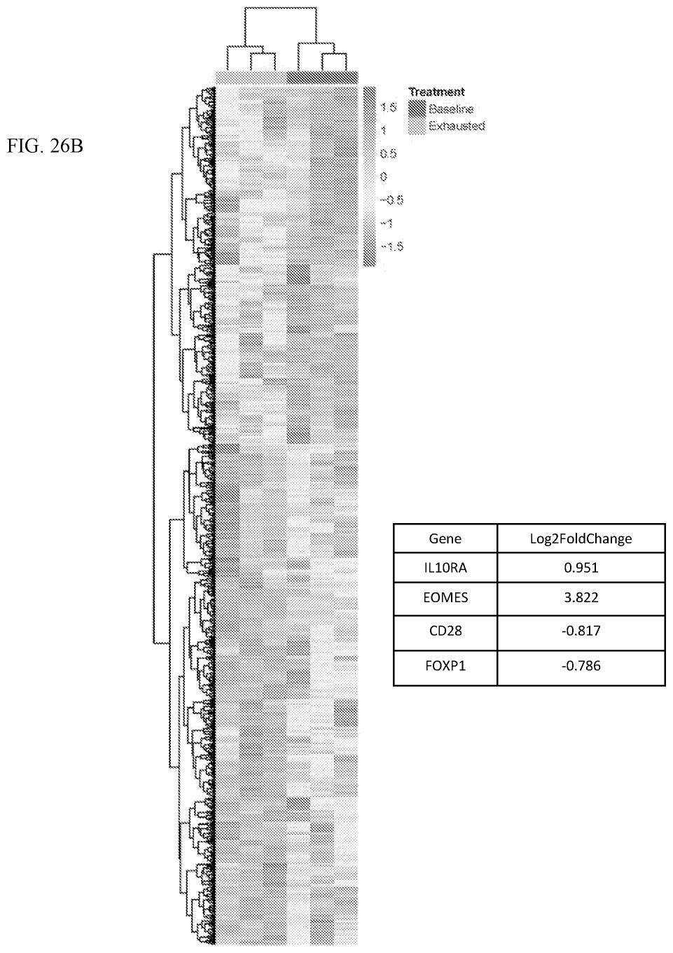

Leukemic EVs carry an inhibitory microRNA cargo. High levels of EOMES promoted T cell exhaustion (FIGS. 26A-26B). T cells were dysfunctional on Day 7. RNA-seq and ATAC-seq were performed to confirm changes in expression. Z-scores are calculated based on the data set's correlation with the activated state. Z (standard score)=x (observed value)−mew (mean of the sample)/sigma (SD of the sample).

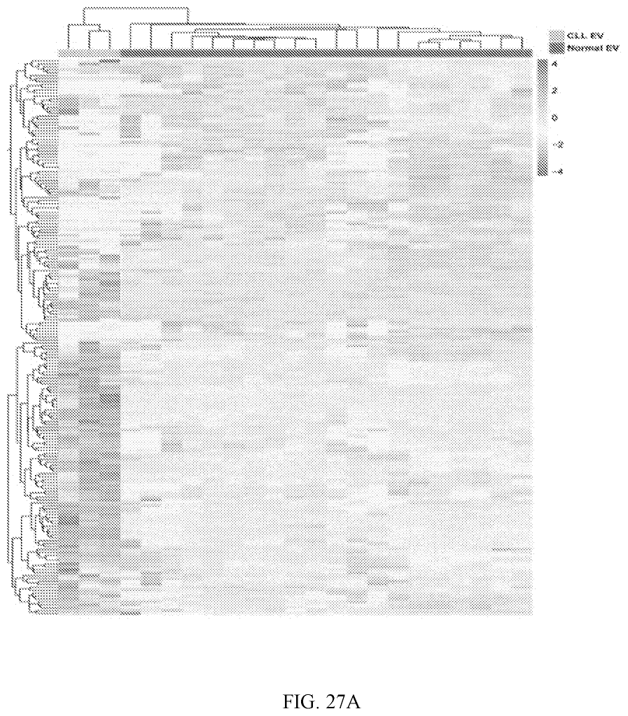

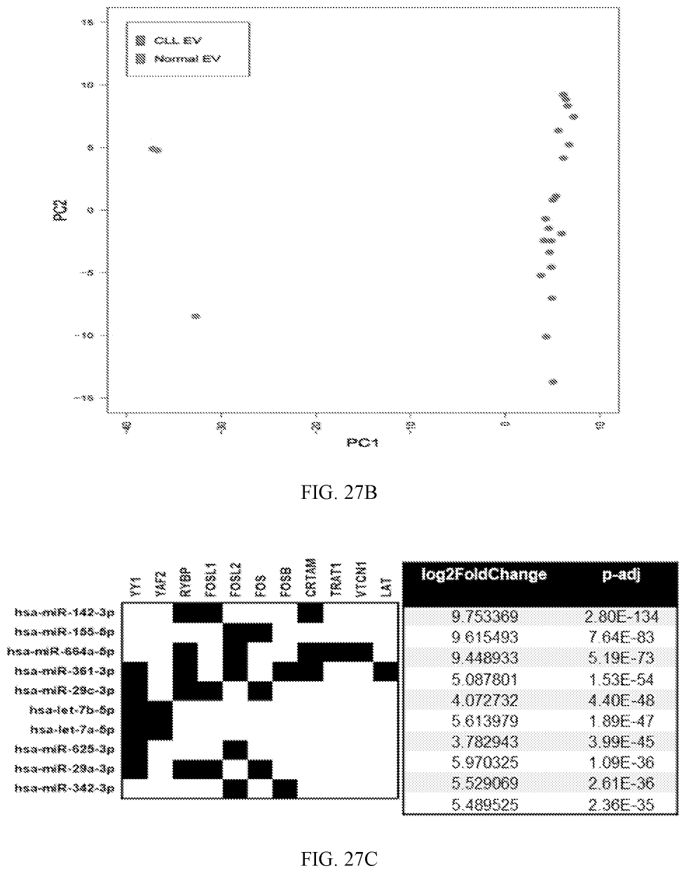

Gene expression analysis was used to compare the microRNA signature of the CLL-EVs to that of normal donors. It was found that 226 microRNA families that were differentially expressed (FIG. 27A). Principal component analysis (PCA) was used to obtain a maximum variance between the individual data points (FIG. 27B). This is done in an unsupervised manner. The top 10 microRNAs that were significantly upregulated target pathways involved in T cell activation and dysfunction (FIG. 27C).

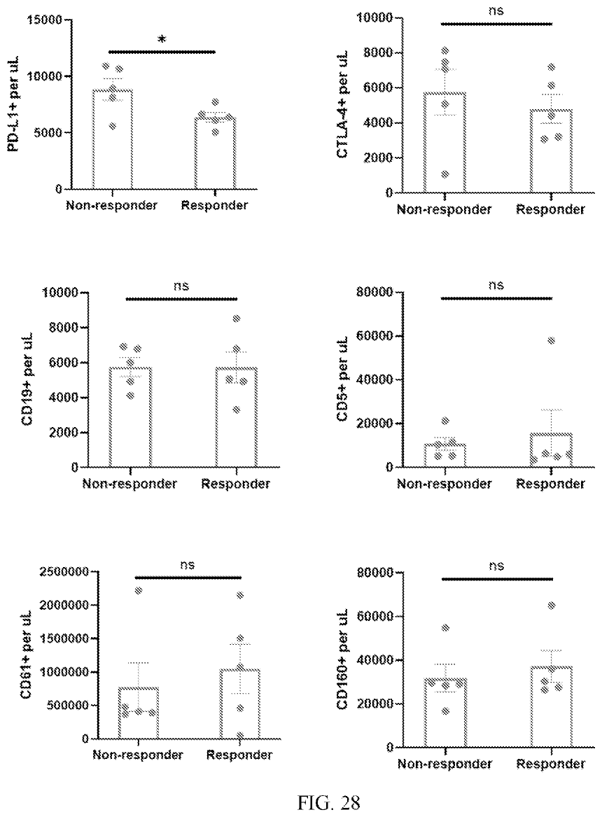

PDL1^{high} EVs are associated with lack of response in patients with lymphoma treated with CART19 cell therapy (FIG. 28).

Together, these results show that CART cells at end of repeated stimulation demonstrate upregulation of transcriptional and epigenetic signature of exhaustion.

### Example 8: MiRNA Isolated from EVs of Responders and Non-Responders to CART19 Cell Therapy in DLBCL

### Methods:

miRNA cargo and PD-L1 levels in samples from CART-treated lymphoma patients were characterized, comparing responders to non-responders and comparing different time points before and during treatment.

EVs were purified from platelet poor plasma using size exclusion chromatography. Fractions surrounding the EV zone were run on a nanoscale flow cytometer to determine which specific fractions carry 80% of the EVs. This was different for each sample depending on the number of EVs in the plasma and the size of those EVs.

Purified EVs were concentrated in order to isolate the miRNA using ultrafiltration, and miRNA was isolated using Qiagen miRNeasy Minipreps.

Differential expression of miRNAs was determined using the miRNA-seq sequence analysis pipeline. miRDeep2 mapper was used to remove adapters and to map reads to the genome. miRDeep2 module was used to map reads against potential miRNA precursors (miRBase). The largest read count was used when there were multiple mappings.

### Results:

### Baseline

Expression levels at baseline were as shown in Table 4. miRNA was excluded where 25% of the samples did not have at least 5 transcripts present.

TABLE 4

| | | | | | | | NR |
|---|---|---|---|---|---|---|---|
| | | log2Fold | | | | | Expression |
| | baseMean | Change | lfcSE | stat | pvalue | padj | Goes |
| hsa-miR-151a-5p | 81.4117 | 5.634812 | 1.57223 | 3.583961 | 0.000338 | 0.008476 | UP |
| hsa-miR-151b | 81.4117 | 5.634812 | 1.57223 | 3.583961 | 0.000338 | 0.008476 | UP |
| hsa-miR-27b-3p | 125.5689 | 5.670775 | 1.597408 | 3.549985 | 0.000385 | 0.008476 | UP |
| hsa-miR-28-3p | 43.52488 | 6.405271 | 1.964552 | 3.260424 | 0.001112 | 0.018356 | UP |
| hsa-miR-125a-5p | 434.6926 | 3.280317 | 1.045463 | 3.13767 | 0.001703 | 0.018924 | UP |
| hsa-miR-584-5p | 50.74065 | −4.50001 | 1.435553 | −3.13469 | 0.00172 | 0.018924 | DOWN |
| hsa-miR-9-5p | 89.74676 | 5.144335 | 1.673642 | 3.073737 | 0.002114 | 0.019932 | UP |
| hsa-miR-29c-3p | 55.46484 | 5.061014 | 1.8041 | 2.805285 | 0.005027 | 0.041474 | UP |

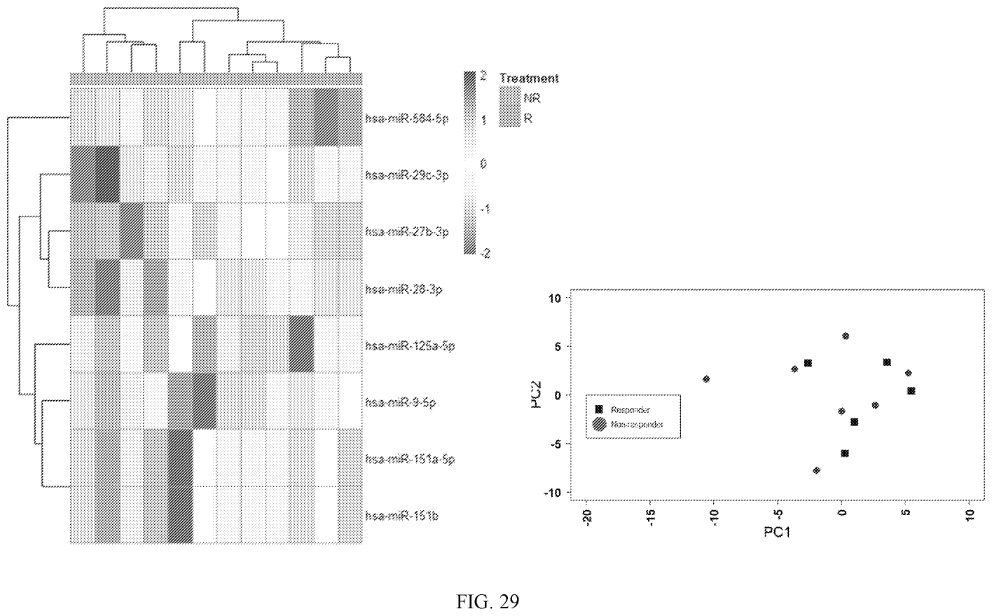

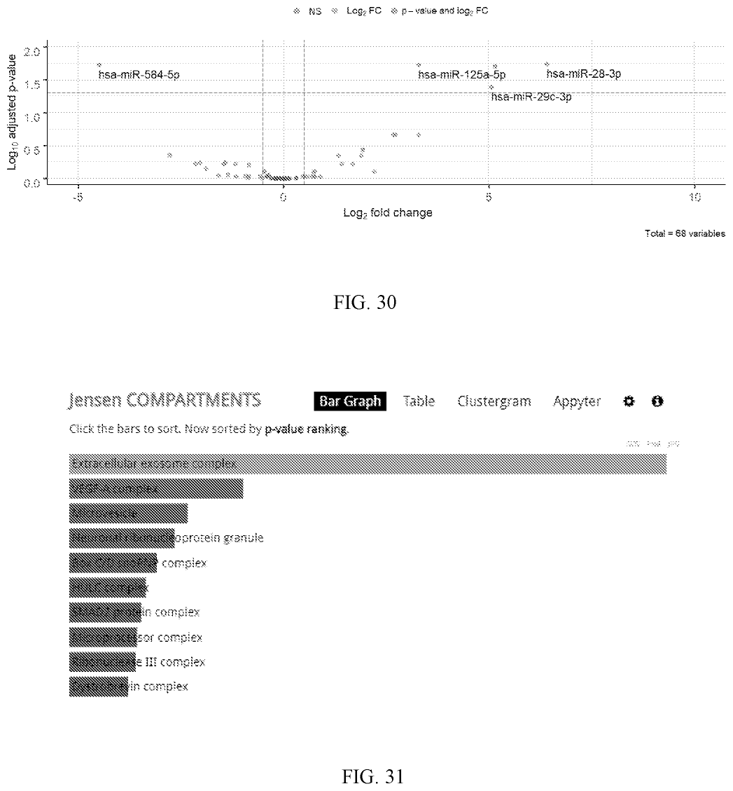

Results of differential expression of miRNAs at baseline are shown in FIGS. 29 to 31. Heatmaps demonstrated significantly altered microRNA between non-responders and responders.

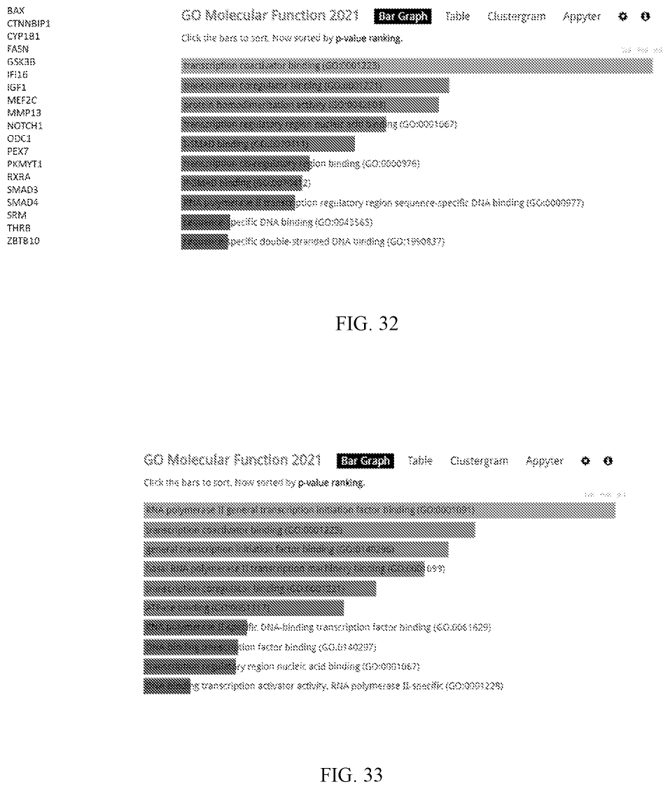

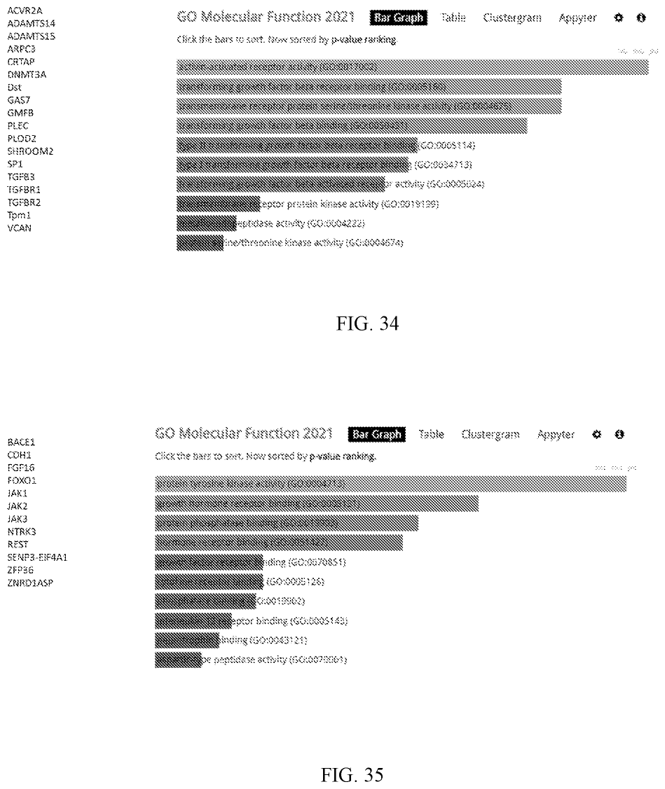

Gene targets of exemplary miRNAs that were upregulated at 1 month are shown in FIGS. 32 to 35. Gene Ontology demonstrated the pathways targeted by specific microRNAs.

### 1 Month

Expression levels at 1 month were as shown in Table 5. miRNA was excluded where 25% of the samples did not have at least 5 transcripts present.

TABLE 5

| | | | | | | | NR |
|---|---|---|---|---|---|---|---|
| | | log2Fold | | | | | expression |
| | baseMean | Change | lfcSE | stat | pvalue | padj | goes |
| hsa-let-7b-5p | 34736.53 | 1.172697 | 0.361203 | 3.24664 | 0.001168 | 0.051965 | UP |
| hsa-miR-9-5p | 507.4091 | 5.622638 | 1.684703 | 3.337465 | 0.000845 | 0.051965 | UP |
| hsa-let-7c-5p | 1655.532 | 1.775452 | 0.60985 | 2.911292 | 0.003599 | 0.064069 | UP |
| hsa-miR-148b-3p | 151.523 | −5.21269 | 1.717094 | −3.03576 | 0.002399 | 0.064069 | DOWN |
| hsa-miR-499a-5p | 189.5358 | 7.089036 | 2.389298 | 2.966996 | 0.003007 | 0.064069 | UP |
| hsa-miR-126-3p | 12828.07 | 0.897497 | 0.321532 | 2.791317 | 0.005249 | 0.077866 | UP |

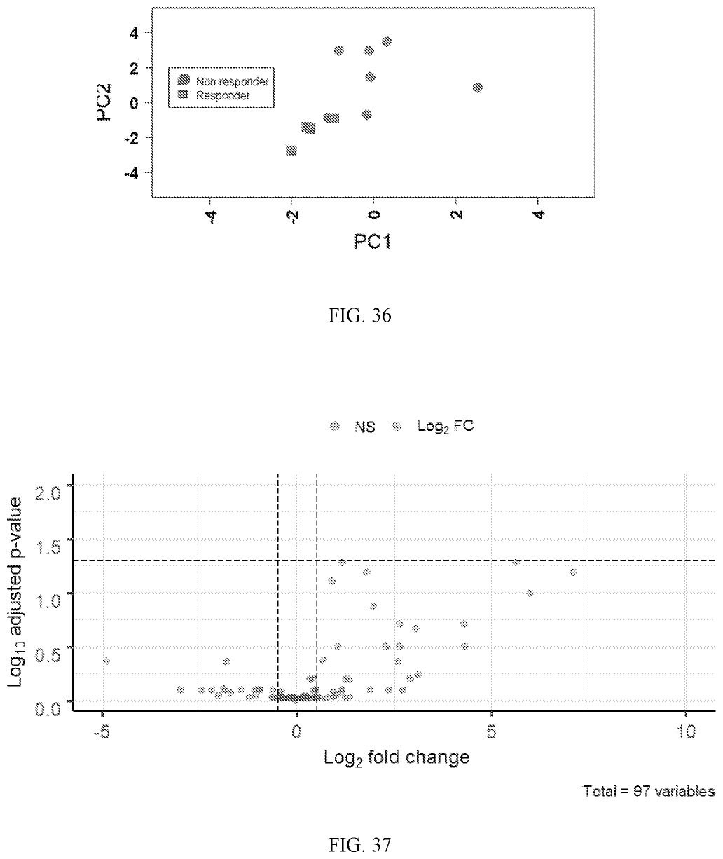

Results of differential expression of miRNAs at 1 month are shown in FIGS. 36 and 37. Principal component analysis and volcano plot demonstrated significantly altered genes in non-responders compared to responders.

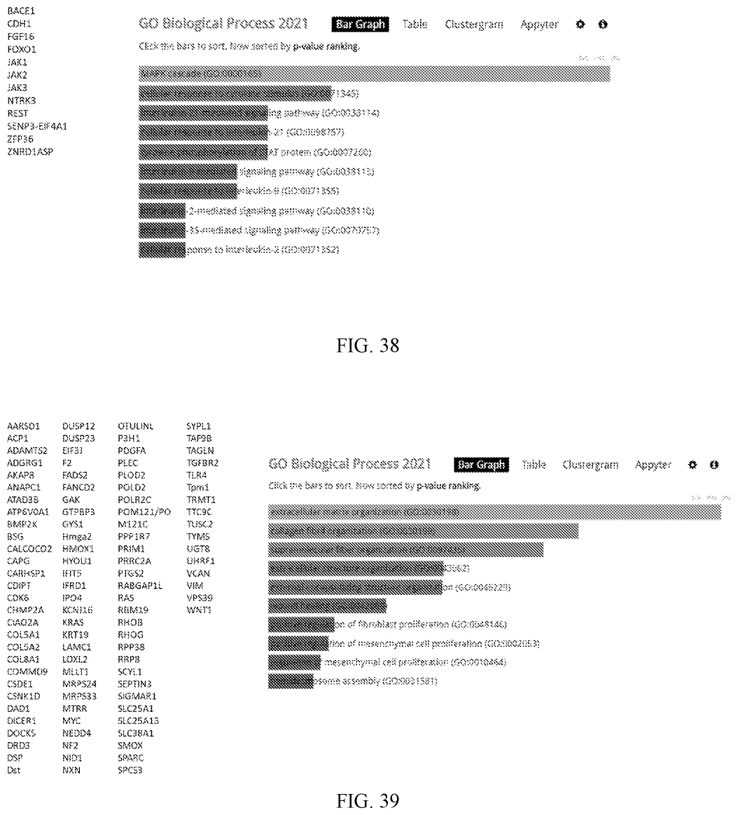

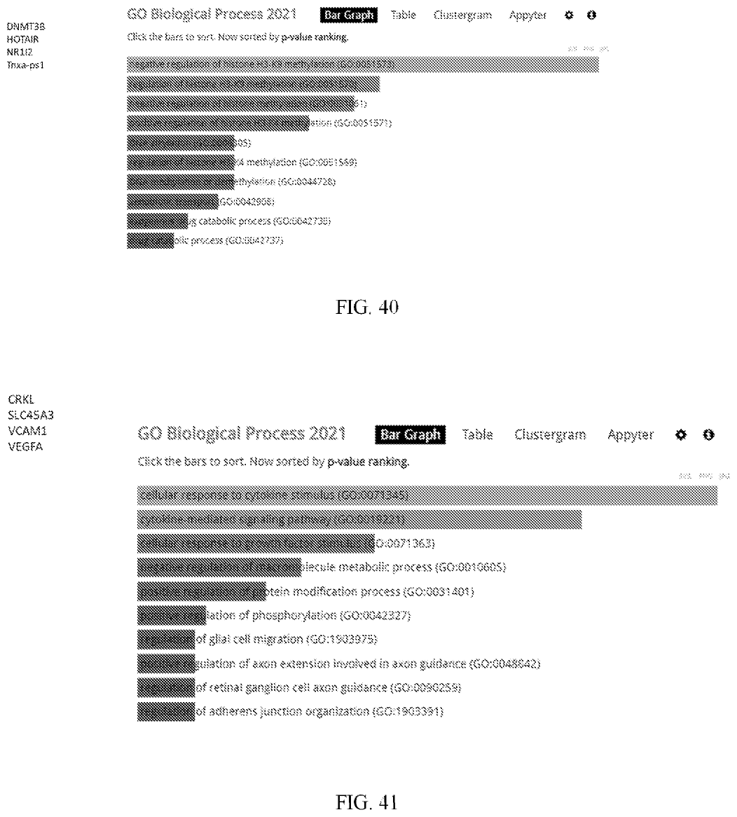

Gene targets of exemplary miRNAs that were upregulated at baseline are shown in FIGS. 38 to 41. Gene Ontology demonstrated the pathways targeted by specific microRNAs.

### 3 Months

Expression levels at 3 months were as shown in Table 6. miRNA was excluded where 25% of the samples did not have at least 5 transcripts present.

TABLE 6

| | | | | | | | NR |
|---|---|---|---|---|---|---|---|
| | | log2Fold | | | | | expression |
| | baseMean | Change | lfcSE | stat | pvalue | padj | goes |
| hsa-miR-184 | 350.4662 | 7.660312 | 1.518975 | 5.043081 | 4.58E−07 | 3.57E−05 | UP |
| hsa-miR-409-3p | 58.57177 | −8.00575 | 2.003577 | −3.99573 | 6.45E−05 | 0.002515 | DOWN |
| hsa-miR-143-3p | 3162.174 | 5.912683 | 1.675415 | 3.529086 | 0.000417 | 0.010842 | UP |
| hsa-miR-125b-5p | 297.9412 | −4.97992 | 1.483738 | −3.35633 | 0.00079 | 0.015221 | DOWN |
| hsa-miR-193a-5p | 72.11561 | 6.429711 | 1.949908 | 3.297443 | 0.000976 | 0.015221 | UP |
| hsa-miR-1294 | 79.79363 | 5.227558 | 1.805653 | 2.895106 | 0.00379 | 0.049274 | UP |

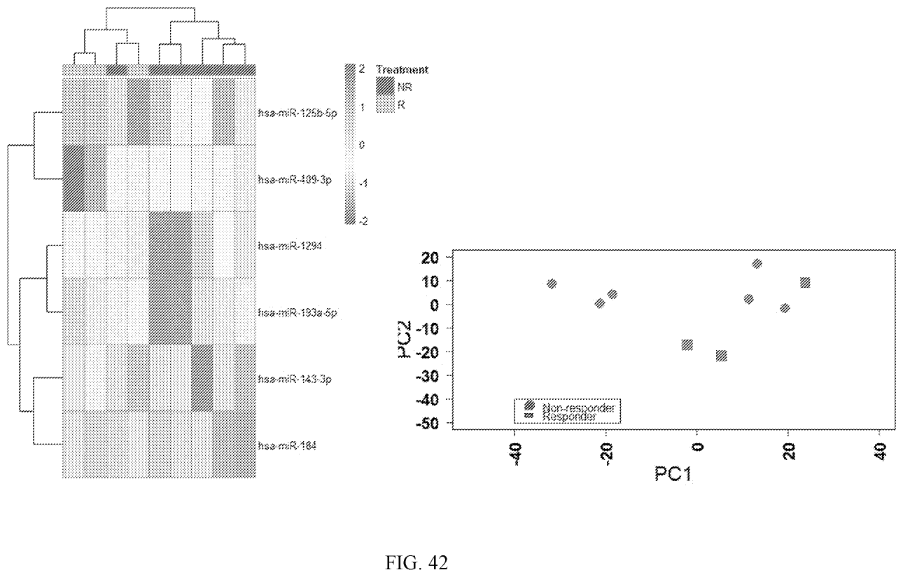

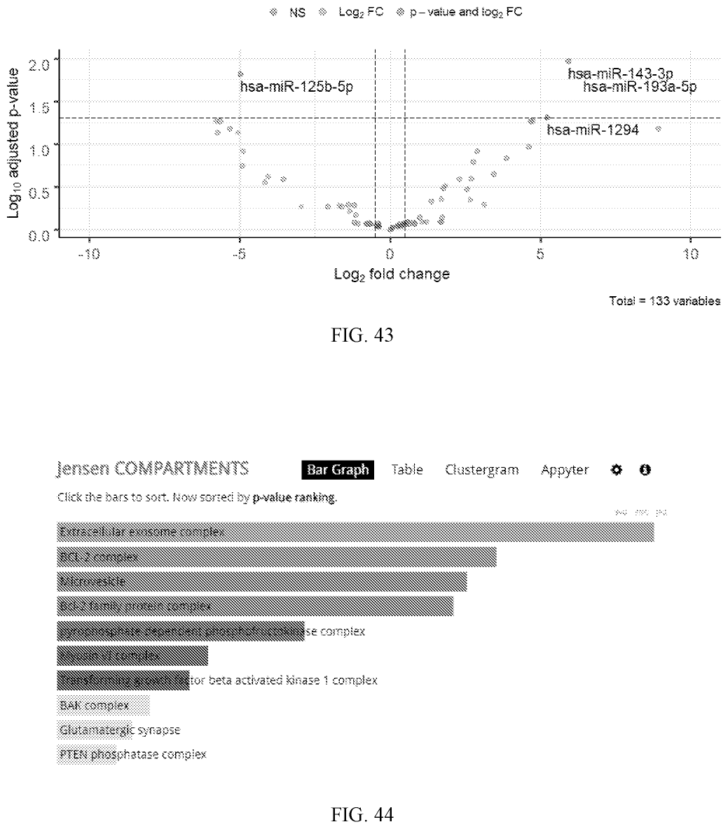

Results of differential expression of miRNAs at 3 months are shown in FIGS. 42 to 44. Principal component analysis and volcano plot demonstrated significantly altered genes in non-responders compared to responders.

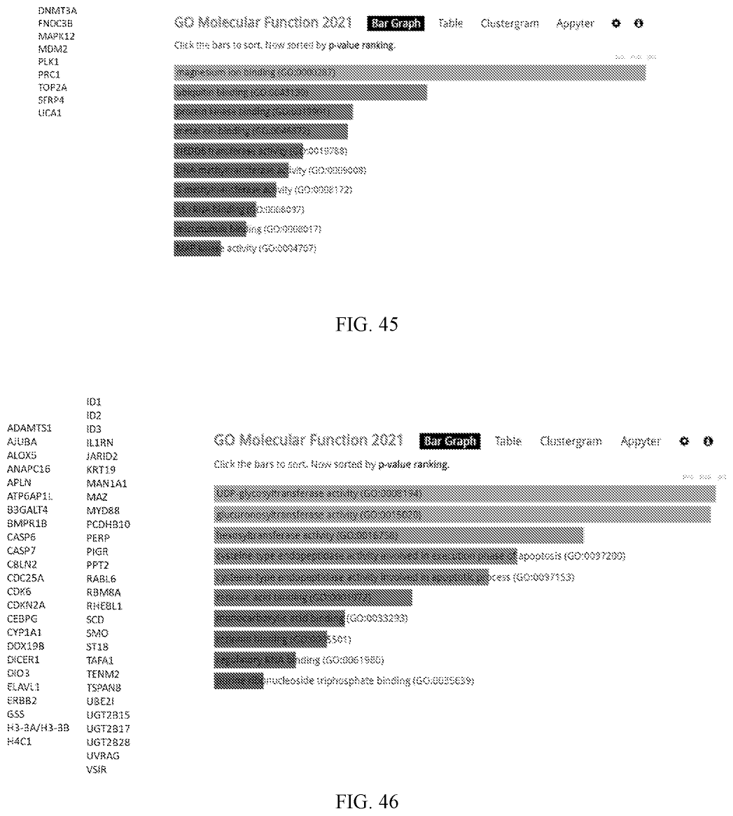

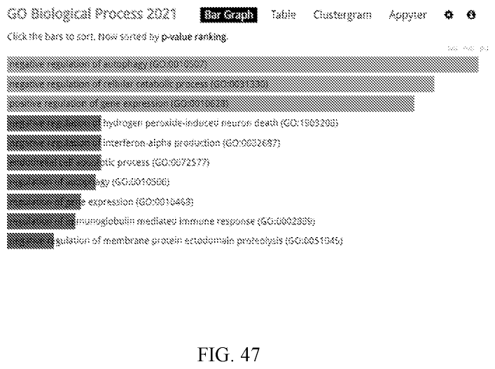

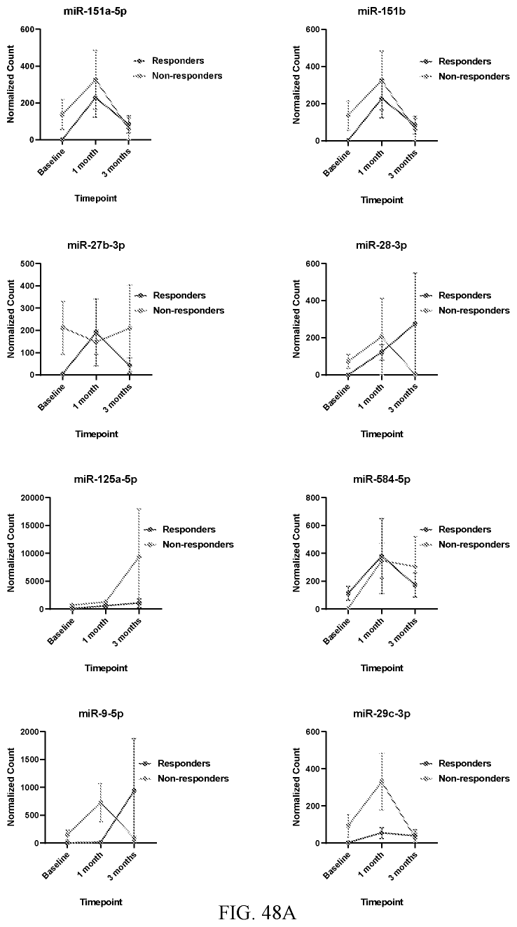

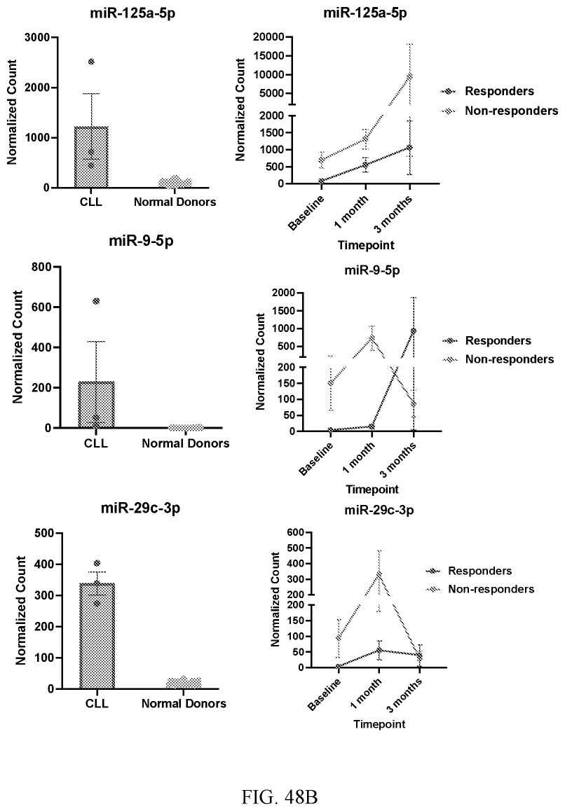

Gene targets of exemplary miRNAs that were upregulated at 3 months are shown in FIGS. 45 to 47. Gene Ontology demonstrated the pathways targeted by specific microRNAs.

Together these results demonstrated no significantly different miRNA overlap between baseline and 3 month time points.

### NFKB Target Genes

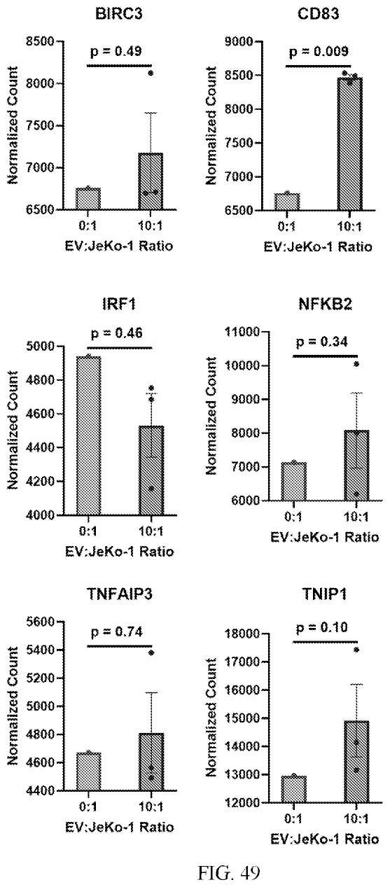

Expression of NFKB target genes was evaluated in JeKo-1 cells that were co-cultured with CLL-EVs (FIG. 49).

| | baseMean | log2FoldChange | lfcSE | stat | pvalue | padj |
|---|---|---|---|---|---|---|
| BIRC3 | 7004.356 | 0.094311 | 0.136277 | 0.692052 | 0.488905 | 0.999807 |
| CD83 | 7992.359 | 0.338937 | 0.13028 | 2.601613 | 0.009279 | 0.394033 |
| IRF1 | 4595.799 | −0.11404 | 0.154167 | −0.73969 | 0.459487 | 0.999807 |
| NFKB2 | 7788.88 | 0.191183 | 0.199609 | 0.957787 | 0.33817 | 0.999807 |
| TNFAIP3 | 4747.657 | 0.057377 | 0.174675 | 0.328478 | 0.742551 | 0.999807 |
| TNIP1 | 14274.84 | 0.207532 | 0.124961 | 1.660772 | 0.096759 | 0.870489 |

| | JeKo1.10.1.EV13 | JeKo1.10.1.EV4 | JeKo1.10.1.EV5 | JeKo1.NoEV |
|---|---|---|---|---|
| BIRC3 | 6712 | 6698 | 8124 | 6759 |
| CD83 | 8390 | 8535 | 8492 | 6760 |
| IRF1 | 4686 | 4158 | 4753 | 4941 |
| NFKB2 | 10052 | 6201 | 8007 | 7138 |
| TNFAIP3 | 5381 | 4492 | 4563 | 4671 |
| TNIP1 | 14147 | 13166 | 17437 | 12968 |

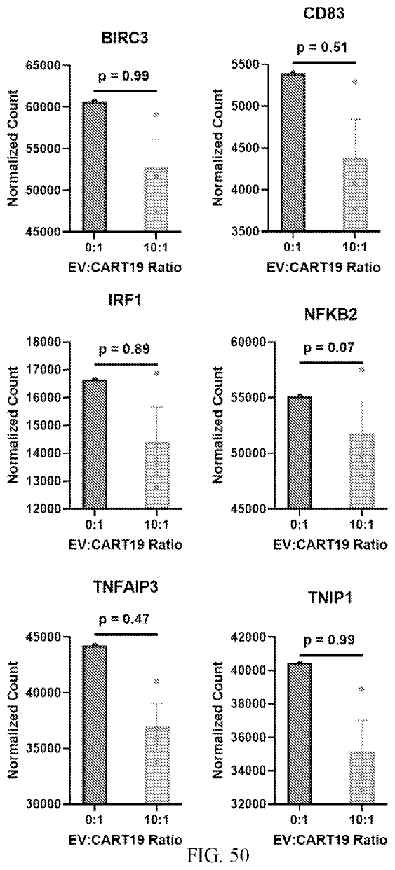

Expression of NFKB target genes was also evaluated in CART19 that were co-cultured with CLL-EVs (FIG. 50).

| | baseMean | log2FoldChange | lfcSE | stat | pvalue | padj |
|---|---|---|---|---|---|---|
| BIRC3 | 49357.77 | 0.000753 | 0.081834 | 0.009196 | 0.992663 | 1 |
| CD83 | 3931.39 | −0.10534 | 0.158689 | −0.66382 | 0.506807 | 1 |
| IRF1 | 14874.6 | −0.01094 | 0.081652 | −0.13396 | 0.893437 | 1 |
| NFKB2 | 48598.58 | 0.113153 | 0.062843 | 1.800568 | 0.071771 | 1 |
| TNFAIP3 | 36977.36 | −0.05714 | 0.078764 | −0.72549 | 0.468153 | 1 |
| TNIP1 | 35289.74 | 0.001329 | 0.080273 | 0.016562 | 0.986786 | 1 |

| | CART19.1.1.EV13 | CART19.1.1.EV4 | CART19.1.1.EV5 | CART19.10.1.EV13 |
|---|---|---|---|---|
| BIRC3 | 57781 | 41246 | 54930 | 47435 |
| CD83 | 5261 | 3408 | 4884 | 3769 |
| IRF1 | 18878 | 15069 | 19443 | 13577 |
| NFKB2 | 60427 | 49516 | 58312 | 49820 |
| TNFAIP3 | 47513 | 35217 | 41203 | 36026 |
| TNIP1 | 44202 | 31257 | 39065 | 33687 |

| | CART19.10.1.EV4 | CART19.10.1.EV5 | CART19.NoEV | CART19.Only |
|---|---|---|---|---|
| BIRC3 | 51589 | 59105 | 60691 | 34724 |
| CD83 | 4073 | 5294 | 5395 | 440 |
| IRF1 | 12765 | 16874 | 16652 | 9856 |
| NFKB2 | 47960 | 57549 | 55141 | 20993 |
| TNFAIP3 | 33752 | 41001 | 44249 | 26550 |
| TNIP1 | 32832 | 38887 | 40450 | 31504 |

These results demonstrate that NFKB genes were targets for the upregulated microRNAs.

TABLE 7
Baseline miRNA integrated with 10:1 CLL-EV:CART19 in IPA - Potential Targets of miR-125ab.

| | | | | | CLL- | |
|---|---|---|---|---|---|---|
| | | | | | EV:CART19 | |
| | | | | | Expr Log | |
| ID | Counts | Source | Confidence | ID | Ratio | Pathway |
| miR-125b-5p | 319.757 | miRecords | Experimentally | ST18 | −5.694 | |
| | | | Observed | | | |
| miR-125b-5p | 319.757 | miRecords | Experimentally | TENM2 | −2.889 | |
| | | | Observed | | | |
| miR-125b-5p | 319.757 | Ingenuity Expert | Experimentally | SH3BP4 | 2.272 | Clathrin-mediated Endocytosis |
| | | Findings, | Observed, | | | Signaling |
| | | TargetScan | Moderate | | | |
| | | Human | (predicted) | | | |
| miR-125b-5p | 319.757 | TargetScan | Experimentally | ERBB3 | 2.975 | Agrin Interactions at |
| | | Human, | Observed, | | | Neuromuscular Junction, |
| | | miRecords | Moderate | | | ERB2-ERBB3 Signaling, |
| | | | (predicted) | | | ERBB Signaling, HER-2 |
| | | | | | | Signaling in Breast Cancer, |
| | | | | | | IL-15 Production, Neuregulin |
| | | | | | | Signaling, Sperm Motility |
| miR-125b-5p | 319.757 | miRecords | Experimentally | B3GALT4 | 3.464 | |
| | | | Observed | | | |
| miR-125b-5p | 319.757 | miRecords | Experimentally | PIGR | 4.15 | |
| | | | Observed | | | |
| miR-125b-5p | 319.757 | miRecords | Experimentally | DIO3 | 4.944 | Thyroid Hormone Metabolism I |
| | | | Observed | | | (via Deiodination), Thyroid |
| | | | | | | Hormone Metabolism II (via |
| | | | | | | Conjugation and/or Degradation), |
| | | | | | | Thyronamine and |
| | | | | | | Iodothyronamine |
| | | | | | | Metabolism, TR/RXR Activation |
| miR-125b-5p | 319.757 | TargetScan | Experimentally | IGFBP3 | 4.968 | Growth Hormone Signaling, Hepatic |
| | | Human, | Observed, | | | Fibrosis/Hepatic Stellate Cell |
| | | miRecords | Moderate | | | Activation, IGF-1 Signaling,RAR |
| | | | (predicted) | | | Activation, VDR/RXR Activation |

### Example 9: FOSL2^{high} CART Cells

Healthy donor T cells were isolated from peripheral blood mononuclear cells using negative magnetic selection. T cells were then stimulated using CD3/CD28 beads and expanded in vitro. Twenty-four hours after stimulation, T cells were transduced with lentiviral vectors expressing both CAR19 and FOSL2 cDNA. Beads were removed on Day 6 and T cells were expanded until Day 8. CAR expression was confirmed and measured by flow cytometry using goat anti-mouse IgG antibody. FOSL2 overexpression was confirmed by western blot. FOSL2^{high} CART19 cells were cryopreserved for future experiments.

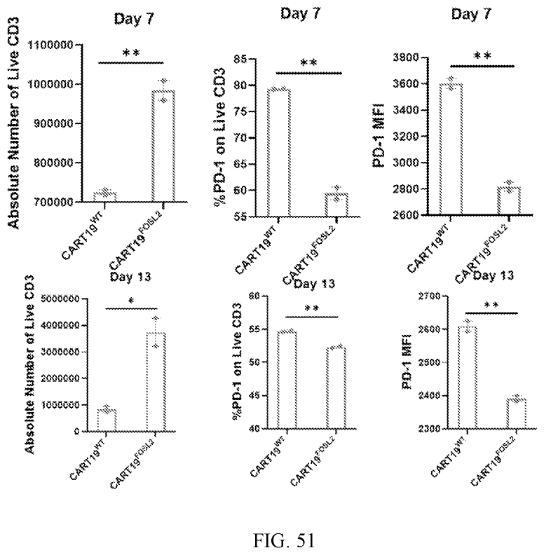

FOSL 2^{high} CART19 cells are less susceptible to inhibition in an in vitro model of CART cell exhaustion (FIG. 51).

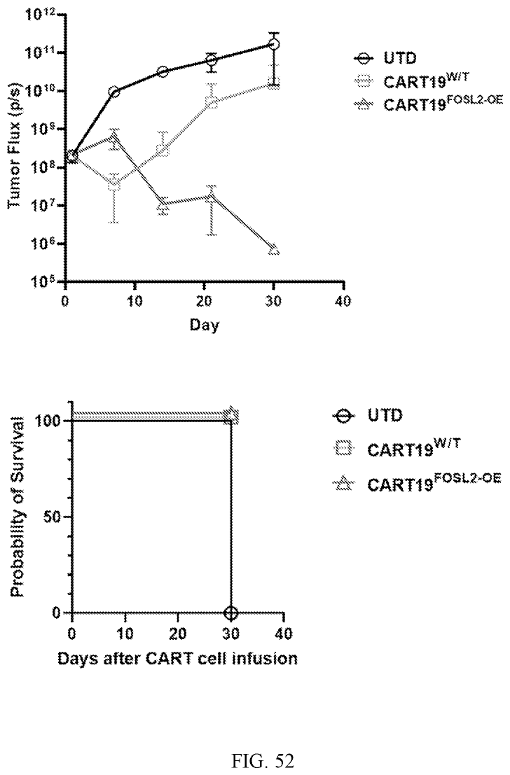

NSG mice were engrafted with the CD19+ luciferase+ JeKo-1 cells. One week following engraftment, mice underwent bioluminescence imaging (BLI) and then randomized to treated with CART19, FOSL2 overexpressing CART19, or control untransduced T cells. Mice were then followed with BLI to monitor disease control. FOSL2 overexpressing CART19 cells result in improved tumor control in xenograft mouse models (FIG. 52).

FOSL2 high CART19 cells exhibited enhanced antigen specific proliferation compared to control CART19, and expressed lower levels of inhibitory receptors. These results indicated less susceptibility to exhaustion. FOSL2^{high} CART19 cells exhibited more potent antitumor activity in JeKo-1 xenografts.

### Example 10: CART Cell Therapy and Small Molecules

CART cell therapy in combination with administration of small molecules that can reduce or eliminate EV production and/or EV trafficking was evaluated.

CART19 cells were cultured in combination with different molecules at the indicated ratios/concentration, and in combination with the luciferase+ JeKo-1 cells at the indicated E:T ratios. Killing was determined after 24 hours using bioluminescence imaging.

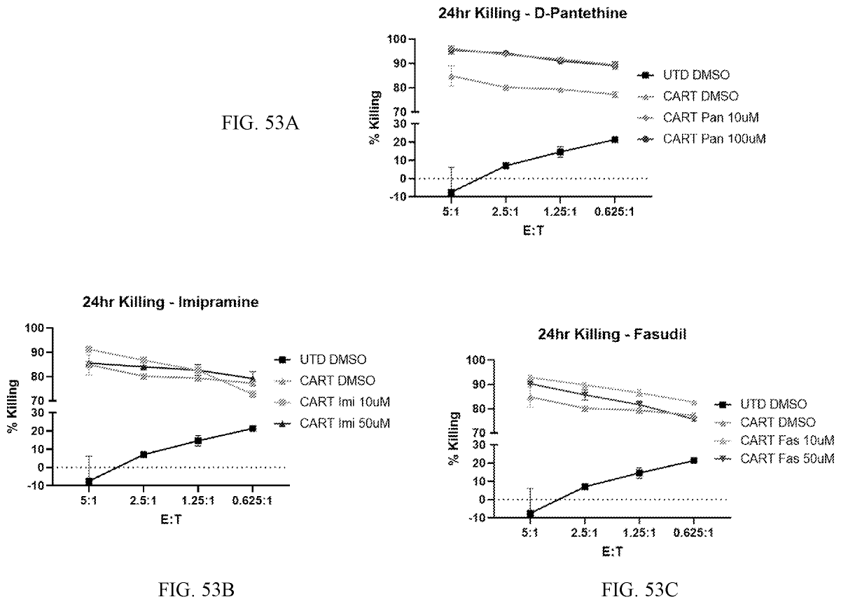

Combination of CART cell therapy with small molecules D-pantethine (FIG. 53A), imipramine (FIG. 53B), and fasudil (FIG. 53C). The combination of CART19 cells with panthethine and fasudil improved CART19 antigen specific killing.

### Example 11: Treating Cancer

A biological sample (e.g., a blood sample such as plasma) is obtained from a human having cancer. The obtained sample is examined for the presence or absence of a PD-L1^{high} EV population. If the absence of a PD-L1^{high} EV population is detected in the sample, then the human is administered a CAR T cell therapy. The administered a CAR T cell therapy can reduce number of cancer cells within the human.

### Example 12: Treating Cancer

A biological sample (e.g., a blood sample such as plasma) is obtained from a human having cancer. The obtained sample is examined for the presence or absence of a PD-L1^{high} EV population. If the presence of a PD-L1^{high} EV population is detected in the sample, then the human is administered one or more chemotherapeutic agents. The administered chemotherapeutic agents can reduce number of cancer cells within the human.

### Example 13: Treating Cancer

A human having leukemia is identified as being likely to respond to one or more immunotherapies (e.g., based, at least in part, on the absence of a PD-L1^{high} EV population in a sample (e.g., a blood sample such as plasma) obtained from the human) is administered a CAR T cell therapy. The administered CAR T cell therapy can reduce number of cancer cells within the human.

### Example 14: Treating Cancer

A human having leukemia identified as not being likely to respond to one or more immunotherapies (e.g., based, at least in part, on the presence of a PD-L1^{high} EV population in a sample (e.g., a blood sample such as plasma) obtained from the human) is administered one or more chemotherapeutic agents. The administered chemotherapeutic agents can reduce number of cancer cells within the human.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-9. (canceled)

10. A method for treating a mammal having cancer, wherein said method comprises:
(a) identifying said mammal as lacking a PD-L1^{high} EV population in a sample obtained from said mammal; and
(b) administering a cancer immunotherapy to said mammal.

11. The method of claim 10, wherein said mammal is a human.

12. The method of claim 10, wherein said cancer is selected from the group consisting of a chronic lymphocytic leukemia, a myeloid leukemia, a non-Hodgkin lymphoma, a Hodgkin lymphoma, a myeloproliferative neoplasm, a breast cancer, a colon cancer, a lung cancer, a pancreatic cancer, a head and neck cancer, a gastrointestinal malignancy, a liver cancer, a cholangiocarcinoma, a skin cancer, a melanoma, and a sarcoma.

13. (canceled)

14. The method of claim 10, wherein said cancer immunotherapy is a CAR T cell therapy.

15. A method for treating a mammal having cancer, wherein said method comprises:
(a) identifying said mammal as having a PD-L1^{high} EV population in a sample obtained from said mammal; and
(b) administering a cancer treatment to said mammal, wherein said cancer treatment is not a cancer immunotherapy.

16. The method of claim 15, wherein said mammal is a human.

17. The method of claim 15, wherein said cancer is selected from the group consisting of a chronic lymphocytic leukemia, a myeloid leukemia, a non-Hodgkin lymphoma, a Hodgkin lymphoma, a myeloproliferative neoplasm, a breast cancer, a colon cancer, a lung cancer, a pancreatic cancer, a head and neck cancer, a gastrointestinal malignancy, a liver cancer, a cholangiocarcinoma, a skin cancer, a melanoma, and a sarcoma.

18. (canceled)

19. The method of claim 15, wherein said cancer treatment comprises administering a chemotherapeutic agent to said mammal.

20. The method of claim 15, wherein said cancer treatment comprises subjecting said mammal to a radiation therapy.

21. A method for treating a mammal having cancer, wherein said method comprises administering a cancer immunotherapy comprising T cells to said mammal, wherein said T cells comprise a nucleic acid encoding a polypeptide that reduces T cell exhaustion.

22. (canceled)

23. A method for treating a mammal having cancer, wherein said method comprises administering a cancer immunotherapy comprising T cells to said mammal, wherein said T cells express a nucleic acid comprising a miRNA target binding site.

24. The method of claim 23, wherein a miRNA is capable of binding to said miRNA target binding site, and wherein said miRNA is selected from the group consisting of let-7, miR-155, miR-185, miR86, miR34a, miR15, miR210, miR142, miR15b, miR125a, and miR130.

25. A method for treating a mammal having cancer, wherein said method comprises administering a cancer immunotherapy comprising T cells to said mammal, wherein the genome of said T cells is modified to lack a miRNA target sequence.

26. The method of claim 25, wherein said miRNA target sequence is a target sequence of a let-7d miRNA.

27. A method for treating a mammal having cancer, wherein said method comprises administering a cancer immunotherapy comprising T cells to said mammal, wherein said T cells comprise a genetic modification to reduce the level of a polypeptide that induces T cell exhaustion.

28. (canceled)

29. A method for treating a mammal having cancer, wherein said method comprises:
(a) administering an agent reduces EV production in said mammal; and
(b) administering a cancer immunotherapy to said mammal.

30. The method of claim 29, wherein said agent is selected from the group consisting of calpeptin, manumycin A, Y27632, D-pantethine, imipramine, fasudil, and GW4869.

31. A method for treating a mammal having cancer, wherein said method comprises:
(a) administering an mTOR inhibitor to said mammal; and
(b) administering a cancer immunotherapy to said mammal.

32. (canceled)

33. A method for treating a mammal having cancer, wherein said method comprises:
(a) administering an HDAC inhibitor to said mammal; and
(b) administering a cancer immunotherapy to said mammal.

34. (canceled)

35. A method for treating a mammal having cancer, wherein said method comprises:
(a) administering a checkpoint blocker to said mammal; and
(b) administering a cancer immunotherapy to said mammal.

36. (canceled)

37. A method for treating a mammal having cancer, wherein said method comprises:
(a) administering a senotherapeutic agent to said mammal; and
(b) administering a cancer immunotherapy to said mammal.

38. (canceled)

39. A method for treating a mammal having cancer, wherein said method comprises:
(a) subjecting said mammal to a therapy that reduces circulating EVs in blood of said mammal; and
(b) administering a cancer immunotherapy to said mammal.

40. The method of claim 39, wherein said therapy that reduces circulating EVs in blood of said mammal is selected from the group consisting of plasma exchange, ultrafiltration, and administration of a plasma adsorbent.

41-44. (canceled)


SEQUENCE LISTING (10 sequences)

SEQ ID NO: 1 - RNA, 87 nt, Artificial Sequence
  Description of Artificial Sequence Synthetic oligonucleotide
ccuaggaagagguaguagguugcauaguuuuagggcagggauuuugcccacaaggagguaacuauacgaccugcugccuuucuuagg

SEQ ID NO: 2 - RNA, 82 nt, Homo sapiens
agggggcgagggauuggagagaaaggcaguuccugaugguccccuccccaggggcuggcuuuccucugguccuucccuccca

SEQ ID NO: 3 - RNA, 79 nt, Homo sapiens
cccgggcugagguaggagguuguauaguugaggaggacacccaaggagaucacuauacggccuccuagcuuuccccagg

SEQ ID NO: 4 - RNA, 23 nt, Homo sapiens
aaaggaaaaaaguauagccccac

SEQ ID NO: 5 - RNA, 23 nt, Homo sapiens
acugucggguugaguagccccag

SEQ ID NO: 6 - RNA, 23 nt, Homo sapiens
ggggagcccugcuggcagccccg

SEQ ID NO: 7 - RNA, 23 nt, Homo sapiens
ggccgagcaggcacccagcccca

SEQ ID NO: 8 - RNA, 23 nt, Homo sapiens
ccuugguuugccuguccguauaa

SEQ ID NO: 9 - RNA, 22 nt, Homo sapiens
aggggcuggcuuuccucugguc

SEQ ID NO: 10 - RNA, 22 nt, Homo sapiens
cuauacggccuccuagcuuucc